(19) United States
(12) Patent Application Publication
(10) Pub. No.: US 2010/0197573 A1
(43) Pub. Date: Aug. 5, 2010

(54) Transglutaminase Mediated Conjugation of Growth Hormone

(75) Inventors: Florencio Zaragoza Dorwald, Smorum (DK); Niels Langeland Johansen, Copenhagen O (DK); Lars Fogh Iversen, Holte (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 11/917,364

(22) PCT Filed: Jun. 15, 2006
(86) PCT No.: PCT/EP06/63246

§ 371 Date: Jul. 15, 2008

(30) Foreign Application Priority Data
Jun. 15, 2005 (EP) 05105256.1
Oct. 18, 2005 (EP) 05109667.5

(51) Int. Cl. A61K 38/27; C07K 1/113; C07K 14/61; A61K 38/02; A61P 5/00

(52) U.S. Cl. 514/12; 530/345; 530/399; 514/2

(57) ABSTRACT

A method for PEGylating growth hormone, said method comprising reacting growth hormone with an amine comprising nucleophile which further comprises a first functional group in the presence og TGase to form a transaminated growth hormone, followed by a reaction of said transaminated growth hormone with a PEG which has been functionalised with a second functional group, wherein said first and second functional groups are selected so that they react to form a covalent bond.

### FIELD OF THE INVENTION

The present invention relates to a novel method for post-translational conjugation of growth hormone wherein transglutaminase is used to incorporate a point of attachment at specific positions in the peptide whereto PEG can be selectively attached. Said conjugated growth hormones have altered characteristics and may thus be of use in therapeutic applications.

### BACKGROUND OF THE INVENTION

It is well-known to modify the properties and characteristics of peptides by conjugating groups to the peptide which duly change the properties of the peptide. Such conjugation generally requires some functional group in the peptide to react with another functional group in a conjugating group. Typically, amino groups, such as the N-terminal amino group or the ε-amino group in lysines, have been used in combination with a suitable acylating reagent. It is often desired or even required to be able to control the conjugation reaction, i.e. to control where the conjugating compounds are attached and to control how many conjugating groups are attached. This is often referred to as specificity.

Conjugation of peptides in general has been known for a long time, and U.S. Pat. No. 4,179,337 disclosed more than 20 years ago peptides, and in particular growth hormone conjugated to polyethylene or polypropylene glycols.

Different types of chemistries have been disclosed which are effective in forming a bond between the peptide and the moiety to be conjugated to the peptide. EP 605 963 discloses the grafting of aqueous polymers which form an oxime linkage with an aldehyde group on a protein. None of the natural amino acid comprises an aldehyde, so a hydroxyl group thus has to be oxidized as a first step in the conjugating process. WO 96/41813 discloses polymers which are functionalised with an amino-oxy oxime forming group useful in conjugation reactions. WO 98/05363 discloses a compound comprising a peptide and a water-soluble polymer, wherein the two are covalently bonded through an oxime bond at the N-terminal amino acid residue.

Furthermore, the use of enzymes to enable a more specific conjugation of peptides is known. EP 243 929 discloses the use of proteolytic enzymes, such as carboxypeptidase to incorporate a compound with a functional group in the C-terminus of a peptide, where said functional group can subsequently be used to attach cytotoxic groups, other peptides or reporter groups used to facilitate analysis of the peptide, such as e.g. fluorescent groups. This technique, however, limits the point of attachment to the C-terminal amino acid residue, something which constitute a severe limitation if the C-terminal residue is essential for the activity of the peptide.

Transglutaminase has previously been used to alter the properties of peptides. In the food industry and particular in the diary industry many techniques are available to e.g. cross-bind peptides using transglutaminases. Other documents disclose the use of transglutaminase to alter the properties of physiologically active peptides. EP 950665, EP 785276 and Sato, Adv. Drug Delivery Rev. 54, 487-504 (2002) disclose the direct reaction between peptides comprising at least one Gln and amine-functionalised PEG or similar ligands in the presence of transglutaminase, and Wada, Biotech. Lett. 23, 1367-1372 (2001) discloses the direct conjugation of β-lactoglobulin with fatty acids by means of transglutaminase. The international patent application published as WO2005/070468 discloses the use of transglutaminase to incorporate a handle whereto conjugating groups can be attached.

It is an object of the present invention to provide a method by which growth hormone may be conjugated with PEG at specific positions with a high degree of specificity. The conjugates obtained by this method have improved or alternative characteristics, e.g. pharmacological characteristics, which makes them suitable, particularly in therapy.

### SUMMARY OF THE INVENTION

In one embodiment, the invention relates to a method of covalently attaching a PEG moiety to growth hormone, the method comprising reacting in one or more steps a glutamine residue comprising growth hormone represented by formula [Ia]

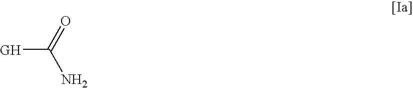

wherein GH represents a growth hormone radical obtained by removing —C(═O)—NH₂ from the side chain of a glutamine residue present in growth hormone, with a nitrogen containing nucleophile of formula [II]

H₂N-D-R—X [II]

wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated growth hormone of formula [IIIa]

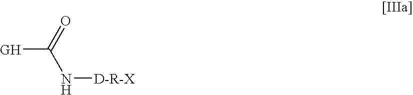

optionally, if X is a latent group, transforming said latent group into —O—NH₂, an aldehyde or a ketone,
said transaminated growth hormone being further reacted with a second compound of formula [IV]

Y—Z [IV]

wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst

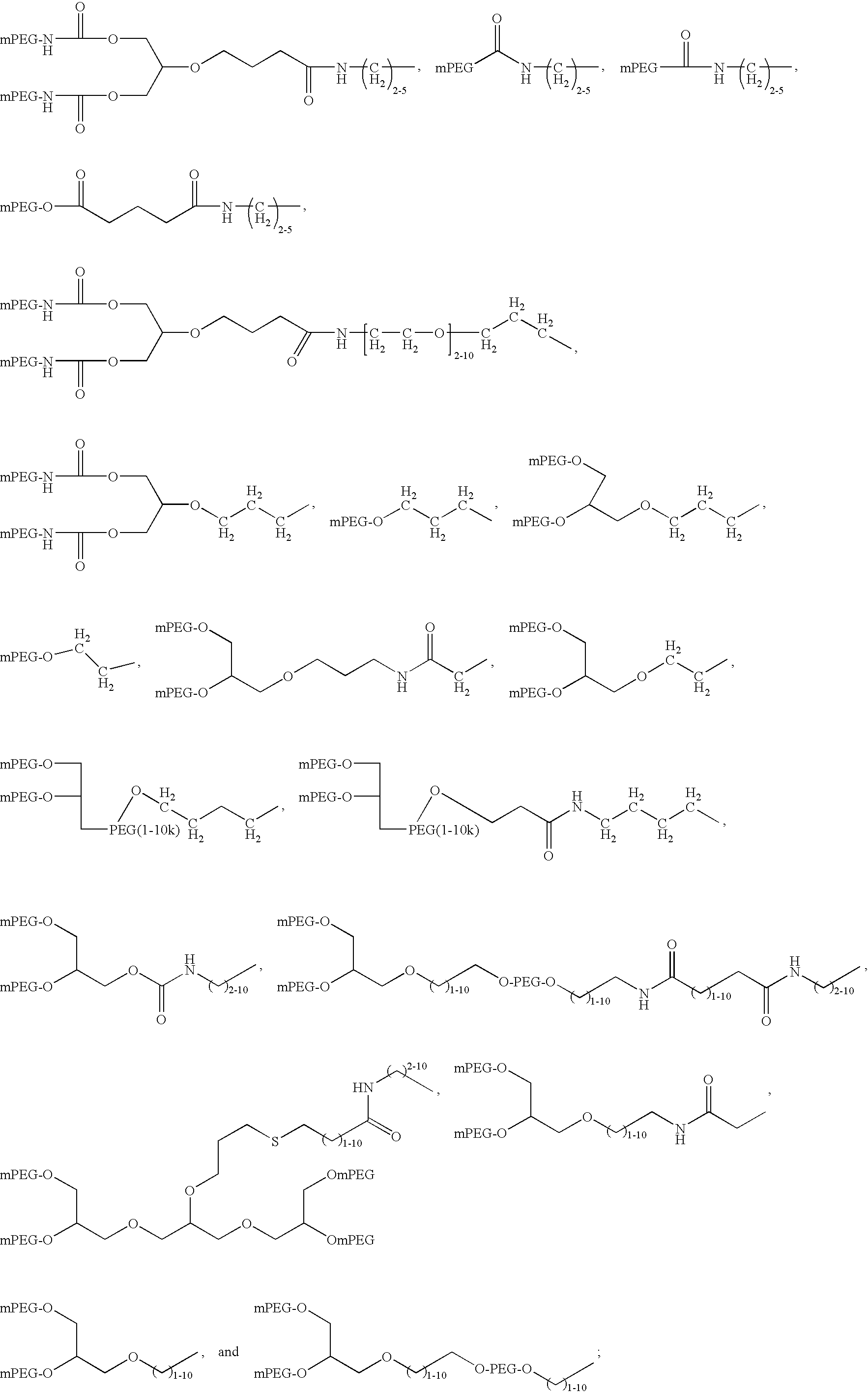

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

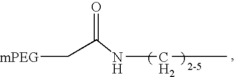

then PEG is 10 kDa PEG to form a PEGylated growth hormone of formula [Va]

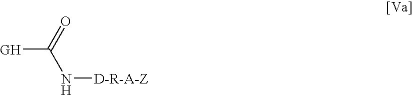

wherein A represents an oxime bond.

In one embodiment, the invention provides compounds of formula [V] or [Va] and any pharmaceutically acceptable salt, prodrug or solvate thereof.

In one embodiment, the invention provides hGH, which has been PEGylated at the position corresponding to position 40 and/or 141 in SEQ ID No. 1.

In a further embodiment, the invention provides compositions comprising compounds of formula [V] or [Va], and in particular pharmaceutical compositions comprising compounds of formula [Va].

In one embodiment, the invention provides the use of compounds of formula [Va] in therapy.

In one embodiment, the invention provides a method of treating diseases which benefit from an increase in the level of the plasma growth hormone level, the method comprising the administration of a therapeutically effective amount of a compound of formula [Va].

In one embodiment, the invention provides the use of a compound of formula [Va] in the manufacture of a medicament for the treatment of diseases benefiting from an increase in the plasma level of growth hormone.

In one embodiment, the invention provides a method for improving or modifying the pharmacological properties of growth hormone, the method comprising PEGylating said growth hormone according to the methods of the present invention.

### BRIEF DESCRIPTION OF THE SEQUENCE LISTING

SEQ ID No. 1 is the amino acid sequence of human growth hormone, also known as 22K-hGH.

### DETAILED DESCRIPTION OF THE INVENTION

The present invention is based on the observation that PEGylation at the N-terminus, and in particular at the C-terminus of hGH gives rise to a much larger decrease in activity than PEGylation at certain amino acids between the two termini, i.e. in-chain PEGylation. In particular, PEGylation at the positions corresponding to positions 40 and/or 141 in a hGH having the sequence of SEQ ID No. 1 gives rise to PEGylated hGH wherein a large proportion of the activity has been retained. Positions 40 and 141 are both glutamine, and, in fact, a hGH having the sequence of SEQ ID No. 1 comprises a further 11 glutamine residues, namely at positions 22, 29, 46, 49, 68, 69, 84, 91, 122, 137, and 181. However, it has surprisingly been found that the use of transglutaminase (TGase), and in particular TGase from Streptoverticillium mobaraenae or Streptomyces lydicus allows a selective PEGylation at positions 40 and/or 141, and that the remaining 11 glutamine residues are left untouched despite the fact that glutamine is a substrate for transglutaminase.

The method of the present invention may also be useful for in-chain PEGylation of any polypeptide comprising one or more glutamine residues, for instance therapeutically interesting polypeptides. This is particularly useful for the pegylation of polypeptides, which comprises more than one glutamine residue, and wherein a site-specific PEGylation is desired, but the method may be used to transglutaminate any glutamine residue comprising polypeptide.

The present invention thus provides a method for covalently attaching PEG to a polypeptide comprising at least one glutamine residue, said method comprising reacting in one or more steps such glutamine residue comprising polypeptide represented by formula [I]

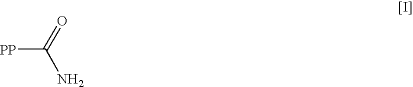

wherein PP represents a polypeptide radical obtained by removing —C(═O)—NH₂ from the side chain of a glutamine residue present in the polypeptide, with a nitrogen containing nucleophile of formula [II]

H₂N-D-R—X [II]

wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated polypeptide of formula [III]

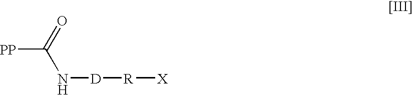

optionally, if X is a latent group, transforming said latent group into —O—NH₂, an aldehyde or a ketone,
said transaminated polypeptide being further reacted with a second compound of formula [IV]

Y—Z [IV]

wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst

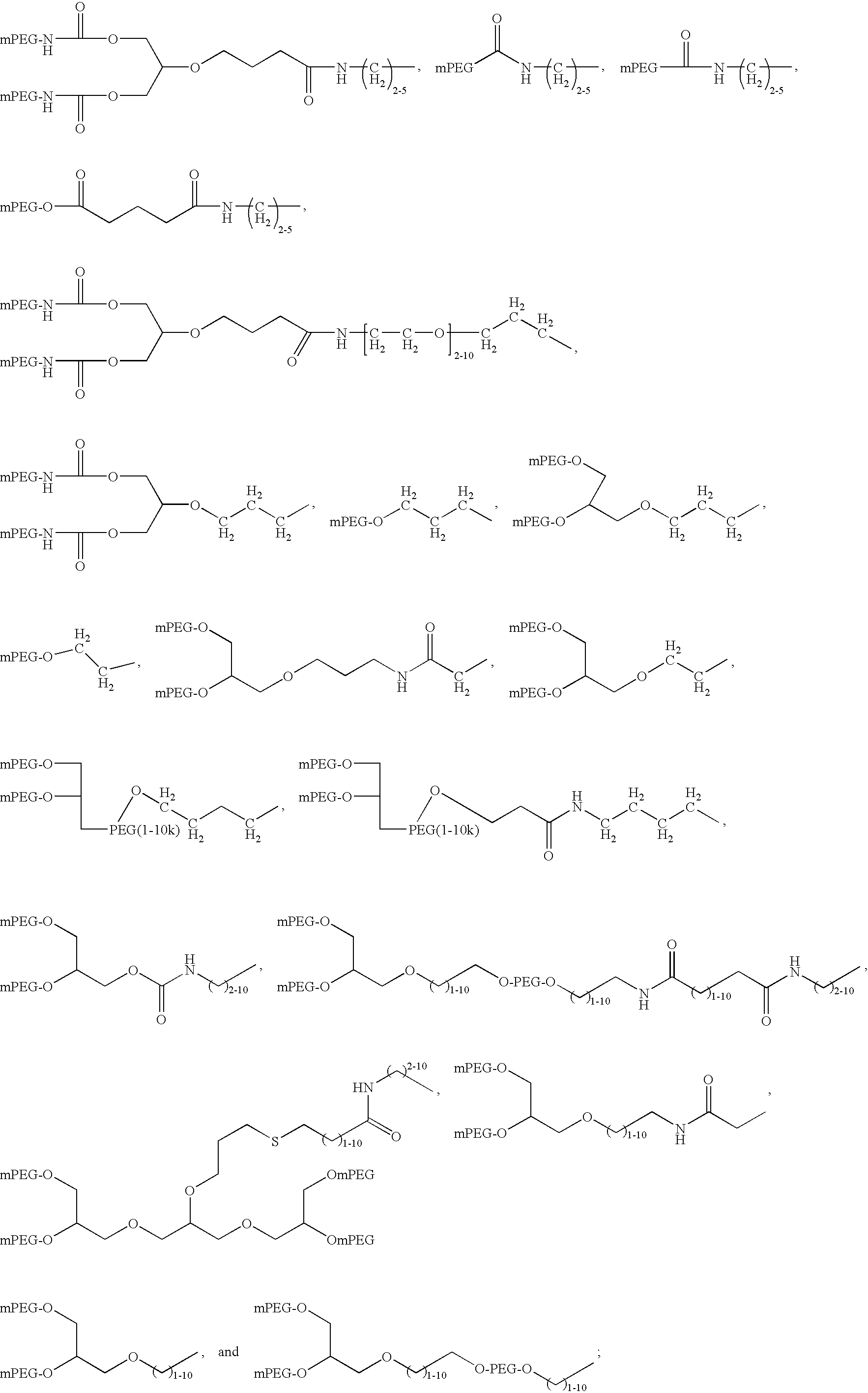

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

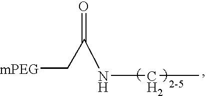

then PEG is 10 kDa PEG to form a PEGylated polypeptide of formula [V]

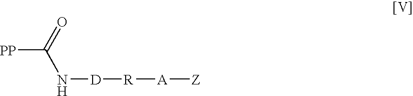

wherein A represents an oxime bond;
or any pharmaceutically acceptable salt, prodrug or solvate thereof.

The term polypeptide as used herein includes any suitable polypeptide and may be used synonymously with the terms peptide and protein, unless otherwise stated or contradicted by context. The term polypeptide herein should generally be understood as referring to any suitable polypeptide of any suitable size and composition (with respect to the number of amino acids and number of associated chains in a protein molecule). Moreover, polypeptides in the context of the inventive methods and compositions described herein may comprise non-naturally occurring and/or non-L amino acid residues, unless otherwise stated or contradicted by context. The polypeptides may also be derivatized. A derivatized polypeptide should generally be understood as referring to a polypeptide in which one or more of the amino acid residues of the polypeptide have been chemically modified (for instance by alkylation, acylation, ester formation, or amide formation) or associated with one or more non-amino acid organic and/or inorganic atomic or molecular substituents and may also or alternatively comprise non-essential, non-naturally occurring, and/or non-L amino acid residues, unless otherwise stated or contradicted by context. Non-limiting examples of such amino acid residues include for instance 2-aminoadipic acid, 3-aminoadipic acid, 6-alanine, 6-aminopropionic acid, 2-aminobutyric acid, 4-aminobutyric acid, 6-aminocaproic acid, 2-aminoheptanoic acid, 2-aminoisobutyric acid, 3-aminoisobutyric acid, 2-aminopimelic acid, 2,4-diaminobutyric acid, desmosine, 2,2′-diaminopimelic acid, 2,3-diaminopropionic acid, N-ethylglycine, N-ethylasparagine, hydroxylysine, allohydroxylysine, 3-hydroxyproline, 4-hydroxyproline, isodesmosine, alloisoleucine, N-methylglycine, N-methylisoleucine, 6-N-methyllysine, N-methylvaline, norvaline, norleucine, ornithine, and statine halogenated amino acids. Polypeptides, which are therapeutically useful and where the therapy using the polypeptides would benefit from for instance an increased retention time, are particularly suitable for use in a method of the present invention.

In one embodiment, the polypeptide is a glutamine residue comprising growth hormone and PP represents a growth hormone radical obtained by removing —C(═O)—NH₂ from the side chain of a glutamine residue present in growth hormone.

The present invention thus provides a method for covalently attaching PEG to growth hormone, the method comprising reacting in one or more steps a glutamine residue comprising growth hormone represented by formula [Ia]

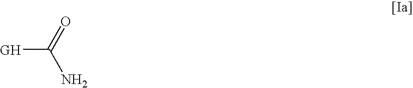

wherein GH represents a growth hormone radical obtained by removing —C(═O)—NH₂ from the side chain of a glutamine residue present in growth hormone, with a nitrogen containing nucleophile of formula [II]

H₂N-D-R—X [II]

wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated growth hormone of formula [IIIa]

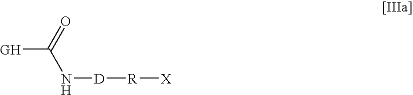

optionally, if X is a latent group, transforming said latent group into —O—NH₂, an aldehyde or a ketone,
said transaminated growth hormone being further reacted with a second compound of formula [IV]

Y—Z [IV]

wherein Y represents —O—NH₂, aldehyde, ketone; and
Z represents a moiety selected amongst

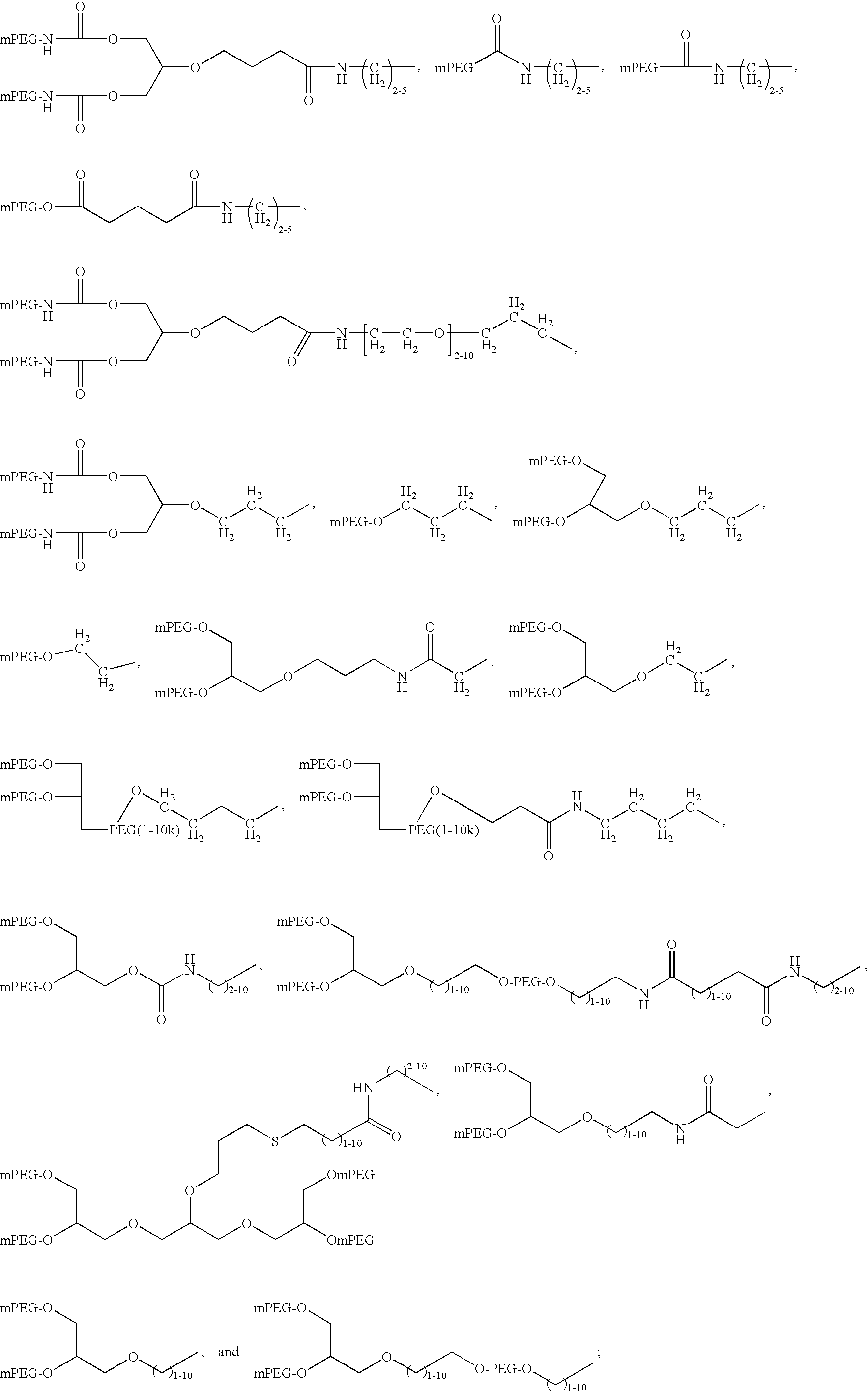

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

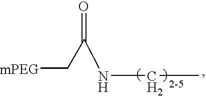

then PEG is 10 kDa PEG to form a PEGylated growth hormone of formula [Va]

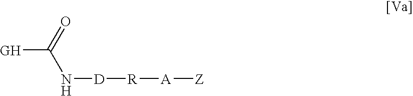

wherein A represents an oxime bond;
or any pharmaceutically acceptable salt, prodrug or solvate thereof.

In the present context, “growth hormone” (GH) is intended to indicate a protein which exhibits growth hormone activity as determined in assay I herein. A protein which exhibits an activity above 20%, such as above 40%, such as above 60%, such as above 80% of that of hGH in said assay is defined as a growth hormone.

In the present context, the term “transamination” and related terms are intended to indicate a reaction where nitrogen in the side chain of glutamine is exchanged with nitrogen from another compound, in particular nitrogen from another nitrogen containing nucelophile.

The term “radical” or “biradical” is intended to indicate a molecular fragment with one or two unpaired electrons, respectively. Such a fragment may be formally generated by removing one (e.g., a hydrogen) or two atoms or groups of atoms (e.g., a hydroxyl group) by homolytic bond cleavage, i.e. a bond cleavage, in which each of the two resulting fragments contains one of the two electrons which formed the original bond. As used herein, “hGH(141)” means a radical formed by formal removal of the CONH₂-group from glutamine(141) in hGH, “hGH(40)” means a radical formed by formal removal of the CONH₂-group from glutamine(40) in hGH, and “hGH(40,141)” means a radical formed by formal removal of the CONH₂-groups from glutamine(40) and glutamine(141) in hGH. hGH(40/141) means a radical formed by formal removal of the CONH₂-groups from glutamine(40) and/or glutamine(141) in hGH, encompassing mixtures of two or more of hGH(40), hGH(141), and (hGH(40,141).

The term “alkylene” is intended to indicate bi-radical of a saturated, linear, branched and/or cyclic hydrocarbon. Unless specified with another number of carbon atoms, the term is intended to indicate hydrocarbons with from 2 to 6 (both included) carbon atoms, such as 2 to 5 (both included), such as from 2 to 4 (both included), e.g. from 2 to 3 (both included). Particular examples include ethylene, propylene, butylene, pentylene and hexylene.

The term heteroalkylene is intended to indicate an alkylene as indicated above in which one or more methylene groups have been substituted with —O—. Particular examples include diradicals of polyethylene glycol.

The term “PEG” or “Peg” means a polydisperse or monodisperse diradical of the structure

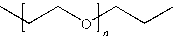

wherein n is an integer larger than 1, and its molecular weight is between approximately 100 and approximately 1,000,000 Da.

The term “mPEG” or “mPeg” means a polydisperse or monodisperse radical of the structure

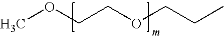

wherein m is an integer larger than 1. Thus, an mPEG wherein m is 90 has a molecular weight of 3991 Da, i.e. approx 4 kDa. Likewise, an mPEG with an average molecular weight of 20 kDa has an average m of 454. Due to the process for producing mPEG these molecules often have a distribution of molecular weights. This distribution is described by the polydispersity index.

The term “polydispersity index” as used herein means the ratio between the weight average molecular weight and the number average molecular weight, as known in the art of polymer chemistry (see e.g. “Polymer Synthesis and Characterization”, J. A. Nairn, University of Utah, 2003). The polydispersity index is a number which is greater than or equal to one, and it may be estimated from Gel Permeation Chromatographic data. When the polydispersity index is 1, the product is monodisperse and is thus made up of compounds with a single molecular weight. When the polydispersity index is greater than 1 it is a measure of the polydispersity of that polymer, i.e. how broad the distribution of polymers with different molecular weights is.

The use of for example “mPEG20000” or “mPEG(20k)” in formulas, compound names or in molecular structures indicates an mPEG residue wherein mPEG is polydisperse and has a molecular weight of approximately 20 kDa.

The polydispersity index typically increases with the molecular weight of the PEG or mPEG. When reference is made to 20 kDa PEG and in particular 20 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 30 kDa PEG and in particular 30 kDa mPEG it is intended to indicate a compound (or in fact a mixture of compounds) with a polydisperisty index below 1.06, such as below 1.05, such as below 1.04, such as below 1.03, such as between 1.02 and 1.03. When reference is made to 40 kDa PEG and in particular 40 kDa mPEG it is intended to indicate a compound (or in fact a mixture of compounds) with a polydisperisty index below 1.06, such as below 1.05, such as below 1.04, such as below 1.03, such as between 1.02 and 1.03.

The term “PEGylated GH” or “PEGylated hGH” is intended to indicate GH or hGH which has been covalently attached to PEG, i.e. it indicates a conjugate comprising GH or hGH and PEG, wherein said GH or hGH and said PEG are covalently bound to each other. Said attachment may be via a linker.

The term “conjugate” as a noun is intended to indicate a modified peptide, i.e. a peptide with a moiety bonded to it to modify the properties of said peptide. As a verb, the term is intended to indicate the process of bonding a moiety to a peptide to modify the properties of said peptide.

The term “oxime bond” is intended to indicate a chemical substructure of the structure —O—N═. In the structural formulas used herein, the oxime bond represented by A in the formula may be positioned in either direction, that is either —O—N═ or ═N—O—. In one embodiment, the direction of the oxime bond in the structural formula given is —O—N═. In another embodiment, the direction of the oxime bond in the structural formula given is ═N—O—.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

The term “prodrug” as used herein is intended to indicate a compound which not or which not necessarily has a therapeutic activity but which upon administration is transformed into a therapeutically active compound by a reaction taking place in the body. Typically such reactions are hydrolysis, e.g. by esterases or oxidations. Examples of prodrugs include biohydrolyzable amides and biohydrolyzable esters and also encompasses a) compounds in which the biohydrolyzable functionality in such a prodrug is encompassed in the compound according to the present invention, and b) compounds which may be oxidized or reduced biologically at a given functional group to yield drug substances according to the present invention. Examples of these functional groups include 1,4-dihydropyridine, N-alkylcarbonyl-1,4-dihydropyridine, 1,4-cyclohexadiene, tert-butyl, and the like.

As used herein, the term “biohydrolyzable amide” is an amide of a drug substance (in casu, a compound according to the present invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable amide is orally absorbed from the gut and is transformed to a compound according to the present invention in plasma. Many examples of such are known in the art and include by way of example lower alkyl amides, α-amino acid amides, alkoxyacyl amides, and alkylaminoalkylcarbonyl amides.

As used herein, the term “biohydrolyzable ester” is an ester of a drug substance (in casu, a compound according to the invention) which either a) does not interfere with the biological activity of the parent substance but confers on that substance advantageous properties in vivo such as duration of action, onset of action, and the like, or b) is biologically inactive but is readily converted in vivo by the subject to the biologically active principle. The advantage is, for example increased solubility or that the biohydrolyzable ester is orally absorbed from the gut and is transformed to a compound according to the present invention in plasma. Many examples of such are known in the art and include by way of example C₁-C₄alkyl esters, C₁-C₄acyloxyalkyl esters, C₁-C₄alkoxyacyloxyalkyl esters, alkoxyacyloxy esters, alkyl acylamino alkyl esters, and choline esters.

Transglutaminase (E.C.2.3.2.13) is also known as protein-glutamine-γ-glutamyltransferase and catalyses the general reaction

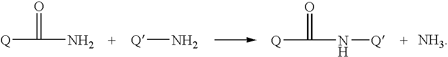

Q-C(O)—NH₂ (amine acceptor) may represent a glutamine residue containing peptide and Q′-NH₂ (amine donor) represents an amine-containing nucleophile. Alternatively, Q-C(O)—NH₂ and Q′-NH₂ may represent an amine acceptor and a lysine-containing peptide, respectively. In the present invention, however, Q-C(O)—NH₂ represents a glutamine residue containing growth hormone and Q′-NH₂ represents an amine-containing nucleophile as indicated above.

A common amine donor in vivo is peptide-bound lysine, and the above reaction then affords cross-bonding of peptides. The coagulation factor Factor XIII is a transglutaminase which effects clotting of blood upon injuries. Different transglutaminases differ from each other, e.g. in what amino acid residues around the glutamine residue (or Gln) are required for the protein to be a substrate, i.e. different transglutaminases will have different Gln-containing peptides as substrates depending on what amino acid residues are neighbours to the Gln residue. This aspect can be exploited if a growth hormone to be modified contains more than one Gln residue. If it is desired to selectively conjugate the growth hormone only at some of the Gln residues present, this selectivity can be obtained be selection of a transglutaminase which only accepts the relevant Gln residue(s) as substrate. Alternatively, one or more amino acid residues close to a Gln may be altered, e.g. by means of genetic engineering to modify the activity of a given transglutaminase to said Gln residue. Glutamine residues may, of course, also be deleted from the growth hormone or substituted with another amino acid to obtain a growth hormone with fewer, or only one glutamine residue to conjugate to.

It is recognised that whether or not a compound is substrate for a given enzyme in principle depends on the reaction conditions, e.g. the time frame, the temperature, etc. Given sufficient time, many compounds not normally regarded as substrates are, in fact, substrates. When it is stated above that for a given transglutaminase some Gln residues may be substrates while others are not it is intended to indicate that “others are not” to an extend where the desired selectivity can still be achieved. If one or more Gln residues, which it is desired to leave unconjugated, are, in fact, a substrate for transglutaminase when in contact with transglutaminase for an extended period of time, selectivity may be achieved by removing or inactivating the transglutaminase after a suitable time.

Examples of useful transglutaminases include microbial transglutaminases, such as e.g. those from Streptomyces mobaraense, Streptomyces cinnamoneum and Streptomyces griseocarneum (all disclosed in U.S. Pat. No. 5,156,956, which is incorporated herein by reference), and from Streptomyces lavendulae (disclosed in U.S. Pat. No. 5,252,469, which is incorporated herein by reference) and Streptomyces ladakanum (JP2003199569, which is incorporated herein by reference). It should be noted that members of the former genus Streptoverticillium are now included in the genus Streptomyces (Kaempfer, J. Gen. Microbiol. 137, 1831-1892 (1991)). Other useful microbial transglutaminases have been isolated from Bacillus subtilis (disclosed in U.S. Pat. No. 5,731,183, which is incorporated herein by reference) and from various Myxomycetes. Other examples of useful microbial transglutaminases are those disclosed in WO 96/06931 (e.g. transglutaminase from Bacilus lydicus) and WO 96/22366, both of which are incorporated herein by reference. Useful non-microbial transglutaminases include guinea-pig liver transglutaminase, and transglutaminases from various marine sources like the flat fish Pagrus major (disclosed in EP-0555649, which is incorporated herein by reference), and the japanese oyster Crassostrea gigas (disclosed in U.S. Pat. No. 5,736,356, which is incorporated herein by reference).

In one embodiment, the glutamine residue comprising growth hormone to be PEGylated is human growth hormone (hGH), which is also known as human somatotropin. Different variants of hGH exist as disclosed e.g. on the public data base SwissProt. It comprises a 26 amino acid signal peptide and a 191 amino acid mature peptide.

In one embodiment, hGH has an amino acid sequence as shown in SEQ ID No. 1 herein (also known as a 22K-hGH).

In one embodiment, the hGH is 20 kDa hGH (or 20K-hGH) as described in J.Clin.Endocrin.Metabol. 89, 1562-1571 (2004) and Endocrine J., 47, S49-S52 (2000). 20 kDa hGH is an hGH with a molecular weight of 20 kDa (20K-hGH), which is secreted by the anterior pituitary gland as a splice variant (alternative splicing of exon 3) and has been reported to account for approximately 5-10% of circulation plasma hGH (Baumann, Endocr. Rev. 12, 424-449 (1991)). 20K-hGH lack 15 amino acids as compared to 22K-hGH (residues 32-46). 22K-hGH is currently approved and used in hGH replacement and pharmacology therapies, however 20K-hGH has never been used in these therapies. 20K-hGH is a full agonist for growth promotion in hypophysectomized rats and dwarf rats, hence as potent as 22K-hGH (Wada et al., Mol. Cellu. Endocr. 113, 99-107 (1997), Ishikawa et al., Growth Horm. IGF Res. 10, 199-206 (2000)). Further, the osteoanabolic effect of 20K-hGH in human osteoblast cells is also equipotent to that of 22K-hGH. In a three arm 24 hours infusion study (in rats) using recombinant 20K-hGH, 22K-hGH and a control group, 20K-hGH had no significant effect on the glucose infusion rate (GIR) in euglycaemic clamps as compared to control. In contrast, 22K-hGH significantly lowered the GIR compared with both 20K-hGH and the control group (Takahashi et al., Growth Horm. IGF Res. 11, 110-116 (2001)). Thus, a peak less (steady) 20K-hGH plasma profile appears to be less diabetogenicity and with reduce insulin-antagonizing action than 22K-hGH. Further, in normal rat 20K-hGH displayed less potency in causing urine retention (oedema formation) than 22K-hGH (Satozawa et al., Growth Horm. IGF Res. 10, 187-192 (2000)). A long-acting growth hormone product will have a plasma profile close to what obtained using an infusion. Hence, using a derivative of 20K-hGH might offer similar desired pharmacology as a 22K-hGH derivative, however with an improved adverse effect profile. In addition, 20K-hGH might have enhanced likelihood to give a long-acting growth hormone product at it has an inhered 30% longer plasma half-life in humans compared to 22K-hGH. The reduced clearance is believed to be due to lowered receptor mediated clearance (Leung et al., Am. J. Physiol Endocr. Metab. 283, 836-843 (2002)). Further, at low concentration (0.1 nM) 20K-hGH was show to be approximately three times more efficient than 22K-hGH to induce IGF-1 expression (Yoshizato et al., Endocr. J. 47, 37-40 (2000)) again indicating a better profile for a long-acting growth hormone product.

In one embodiment, the growth hormone to be PEGylated is a variant of hGH, wherein a variant is understood to be the compound obtained by substituting one or more amino acid residues in the hGH sequence with another natural or unnatural amino acid; and/or by adding one or more natural or unnatural amino acids to the hGH sequence; and/or by deleting one or more amino acid residue from the hGH sequence, wherein any of these steps may optionally be followed by further derivatization of one or more amino acid residues, for instance by pegylation resulting in a di- or multi-peguylated growth hormone variant. In particular, such substitutions are conservative in the sense that one amino acid residue is substituted by another amino acid residue from the same group, i.e. by another amino acid residue with similar properties. Amino acids may conveniently be divided in the following groups based on their properties: Basic amino acids (such as arginine and lysine), acidic amino acids (such as glutamic acid and aspartic acid), polar amino acids (such as glutamine, histidine, cysteine and asparagine), hydrophobic amino acids (such as leucine, isoleucine, proline, methionine and valine), aromatic amino acids (such as phenylalanine, tryptophan, tyrosine) and small amino acids (such as glycine, alanine, serine and threonine).

In one embodiment, the hGH is an hGH in which the glutamine residue in the position corresponding to position 40 in SEQ ID No. 1 has been deleted or substituted with another amino acid. In one embodiment, the hGH is an hGH in which the glutamine residue in the position corresponding to position 141 in SEQ ID No. 1 has been deleted or substituted with another amino acid. In one embodiment, the hGH is an hGH, in which the glutamine residue in the position corresponding to position 40 in SEQ ID No. 1, and the glutamine residue in the position corresponding to position 141 in SEQ ID No. 1 each have been deleted or substituted with another amino acid, and where a glutamine residue is present in another position in the growth hormone. In a further embodiment, said other amino acid is asparagine.

In one embodiment, the glutamine residue comprising growth hormone to be PEGylated has at least 80%, such as at least 85%, such as at least 90%, such as at least 95% such as at least 98% identity with hGH. In one embodiment, said identities to hGH are coupled to at least 20%, such as at least 40%, such as at least 60%, such as at least 80% of the growth hormone activity of hGH as determined in assay I herein.

The term “identity” as known in the art, refers to a relationship between the sequences of two or more proteins, as determined by comparing the sequences. In the art, “identity” also means the degree of sequence relatedness between proteins, as determined by the number of matches between strings of two or more amino acid residues. “Identity” measures the percentage of identical matches between the smaller of two or more sequences with gap alignments (if any) addressed by a particular mathematical model or computer program (i.e., “algorithms”). Identity of related proteins can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity are designed to give the largest match between the sequences tested. Methods to determine identity are described in publicly available computer programs. Preferred computer program methods to determine identity between two sequences include the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well known Smith Waterman algorithm may also be used to determine identity.

For example, using the computer algorithm GAP (Genetics Computer Group, University of Wisconsin, Madison, Wis.), two proteins for which the percent sequence identity is to be determined are aligned for optimal matching of their respective amino acids (the “matched span”, as determined by the algorithm). A gap opening penalty (which is calculated as 3.times. the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci. USA, 89:10915-10919 (1992) for the BLOSUM 62 comparison matrix) is also used by the algorithm.

Preferred parameters for a protein sequence comparison include the following: Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for protein comparisons (along with no penalty for end gaps) using the GAP algorithm.

It should be understood that the above discussed growth hormones to be PEGylated must comprise at least one glutamine residue. The term “glutamine residue” as used herein also includes a glutamine residue analog suitable for transamination. Suitable glutamine residue analogs include, but is not limited to, partially fluorinated, alkylated, or deuterated glutamine residue analogs, or a homolog of a glutamine residue, i.e. a compound resulting from the formal insertion of one, two, or more methylene groups (—CH₂—) into a C—C, C—H, or C—N-bond of glutamine.

Growth hormone may be obtained by standard protein synthetic methods, or growth hormone may be obtained by transfecting a suitable host cell with a DNA encoding the growth hormone of interest. This is within the capabilities of a skilled person. The pegylated growth hormones obtainable by use of a method according to the invention used may also be derivatized by other means than pegylation, if so desired. Such additional derivatization may be performed before, during or after use of the steps of the method of the invention. It is within the skill of a person skilled in the art to determine the timing of such additional derivatisation. The growth hormone used may also already be pegylated at one or more further positions in addition to the site or sites to be pegylated using a method according to the present invention.

In one embodiment, D represents —O—. In another embodiment, D represents a single bond.

The linker R provides proper spacing of the amine of the nucleophile and functional group or the latent functional group to be incorporated in growth hormone. In one embodiment, R represents —(CH₂)₄—CH(NH₂)—CO—NH—CH₂— or —(CH₂)₄—CH(NHCOCH₃)—CO—NH—CH₂—. In one embodiment, R represents C₁₋₆alkylene. In one embodiment, R represents C₁₋₃alkylene. In one embodiment, R represents methylene, ethylene or propylene. In one embodiment, R methylene or propylene.

In one embodiment, Z represents

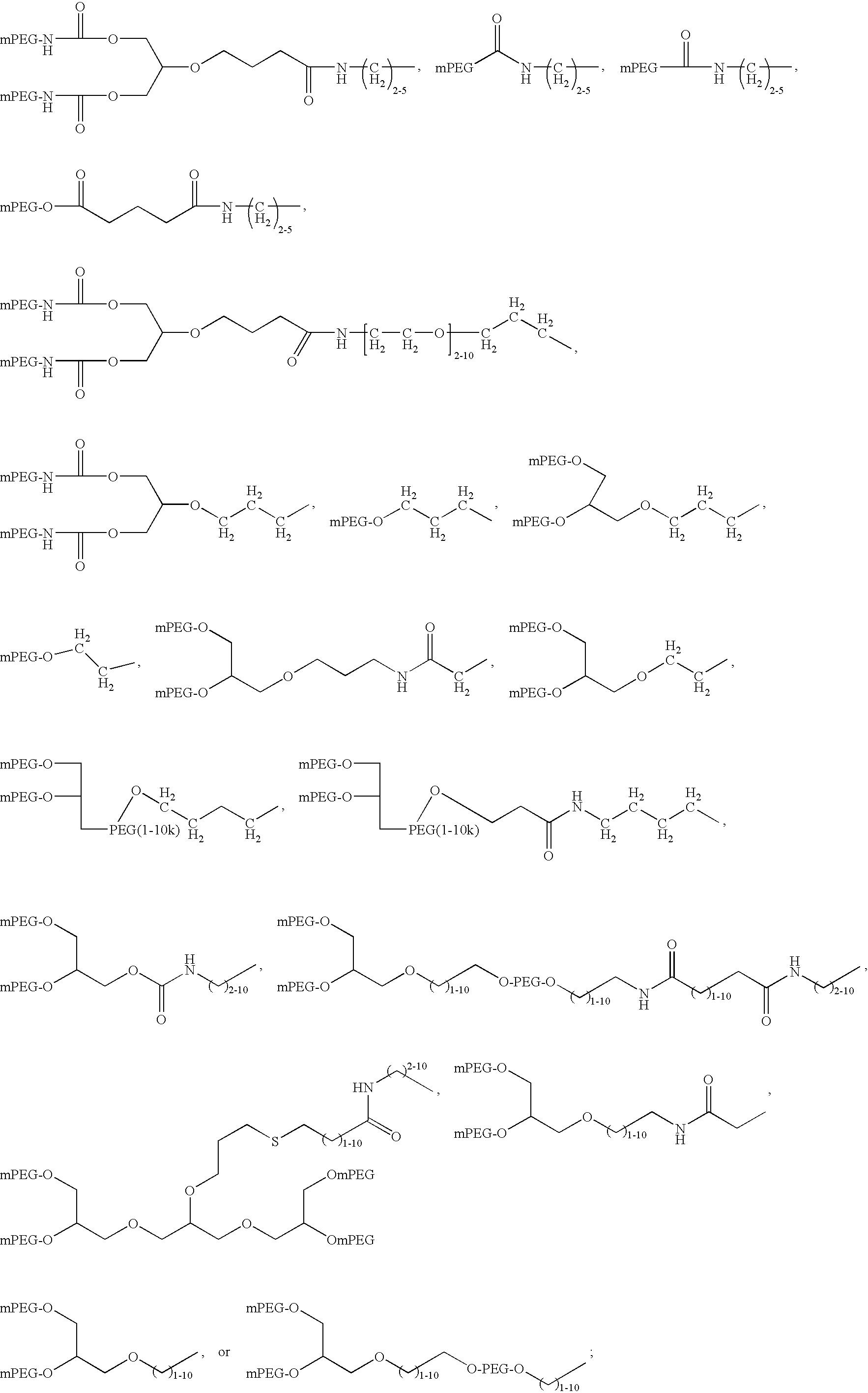

wherein, unless otherwise indicated, mPEG indicates a mPEG with a molecular weight of between 5 kDa and 40 kDa and PEG indicates a PEG with a molecular weight between 1 kDa and 10 kDa.

It is to be understood that the functional group comprised in X may be latent in the sense that it has to be activated prior to the reaction with Y—Z. By way of example, X may comprise a moiety which upon reaction with a suitable reagent is transformed to an aldehyde or a ketone. Examples of such moieties include

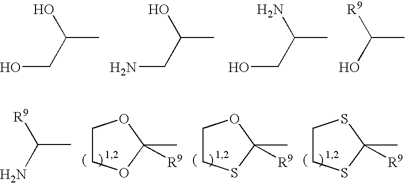

wherein R⁹ represents H, C₁₋₆alkyl, aryl or heteroaryl. Particular examples include methyl, ethyl and propyl. Said moieties may be transformed to an aldehyde or ketone by oxidation with a suitable agent, such as e.g. periodate, or by hydrolysis with an aqueous acid, optionally in the presence of a catalyst, such as copper, silver, or mercury salts.

In the present context, the term “aryl” is intended to indicate a homocyclic aromatic ring radical or a fused homocyclic ring system radical wherein at least one of the rings are aromatic. Typical aryl groups include phenyl, biphenylyl, naphthyl, tetralinyl and the like.

The term “heteroaryl”, as used herein, alone or in combination, refers to an aromatic ring radical with for instance 5 to 7 ring atoms, or to a fused aromatic ring system radical with for instance from 7 to 18 ring atoms, wherein at least on ring is aromatic and contains one or more heteroatoms as ring atoms selected from nitrogen, oxygen, or sulfur heteroatoms, wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl, and the like.

It should also be understood that X and Y must be complementary in the sense that they should be able to react with each other to form an oxime bond. This means that if X is an (or can be activated to be an) aldehyde or ketone, then Y must be an aminoxy. If X is an aminoxy, then Y must be an aldehyde or a ketone. If Y is an aldehyde or a ketone, then X must be an aminoxy. If Y is an aminoxy, then X must be (or must be activated to) an aldehyde or a ketone.

Particular examples of the compound of formula [II] include 1,3-diaminooxy propane and 1,3-diamino-2-propanol. If the latter compound is used, the latent aldehyde group has to be oxidized, e.g. by means of periodate, to be converted to an aldehyde.

In one embodiment, Y represents —O—NH₂ and X represents an aldehyde or a latent group, which may be further reacted to form an aldehyde. In one embodiment, Y represents —O—NH₂ and X represents a ketone or a latent group which may be further reacted to form a ketone. In one embodiment, the compound of formula [II]

H₂N-D-R—X [II]

represents 1,3-diamino-2-propanol, and Y represents —O—NH₂.

In a further embodiment, the compound of formula [IV]

Y—Z [IV]

represents a compound selected from

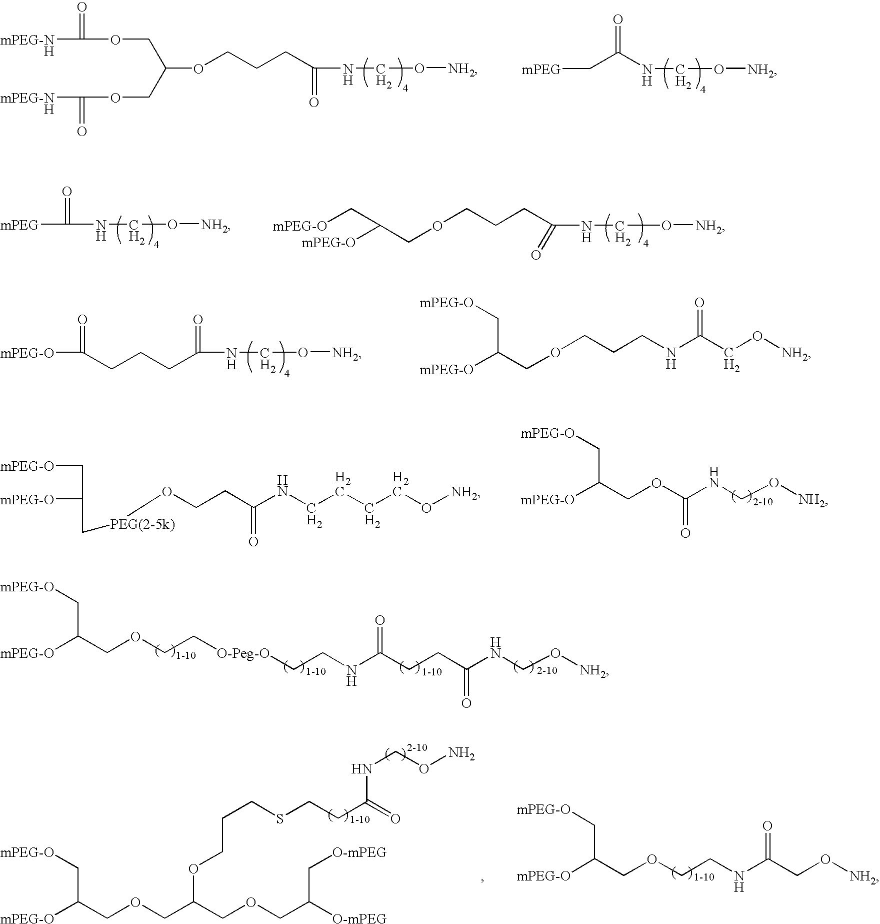

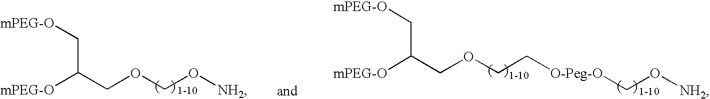

wherein, unless otherwise indicated, mPEG means mPEG with a molecular weight of 10 kDa, 20 kDa or 30 kDa and PEG means PEG with a molecular weight between 2 kDa and 5 kDa.

In one embodiment, Y represents an aldehyde and X represent —O—NH₂ or a latent group which upon further reaction may be transformed into —O—NH₂. In a further embodiment, the compound of formula [II]

H₂N-D-R—X [II]

represents 1,3-diaminooxy propane, and Y represents an aldehyde.

In a further embodiment, the compound of formula [IV]

Y—Z [IV]

represents a compound selected from

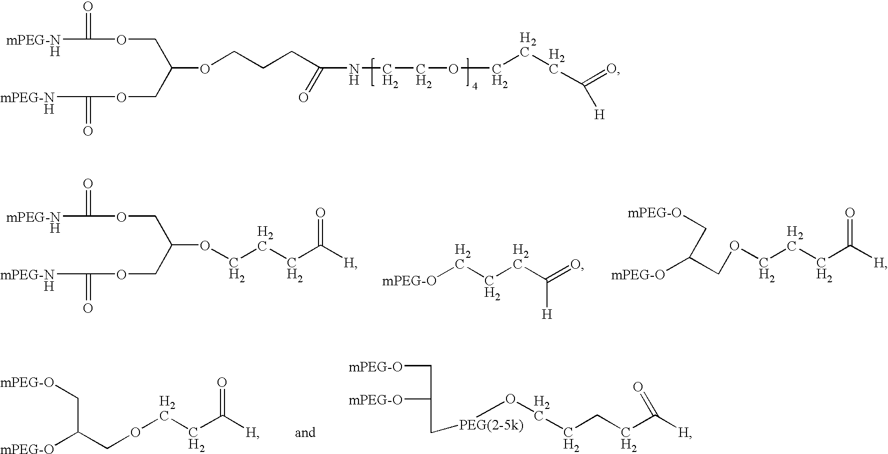

wherein, unless otherwise indicated, mPEG means mPEG with a molecular weight of 10 kDa, 20 kDa or 30 kDa and PEG means PEG with a molecular weight between 2 kDa and 5 kDa.

In one embodiment, Y represents a ketone and X represent —O—NH₂ or a latent group which upon further reaction may be transformed into —O—NH₂. In a further embodiment, the compound of formula [II]

H₂N-D-R—X [II]

represents 1,3-diaminooxy propane, and Y represents an ketone.

In a further embodiment, the compound of formula [IV]

Y—Z [IV]

represents a compound selected from

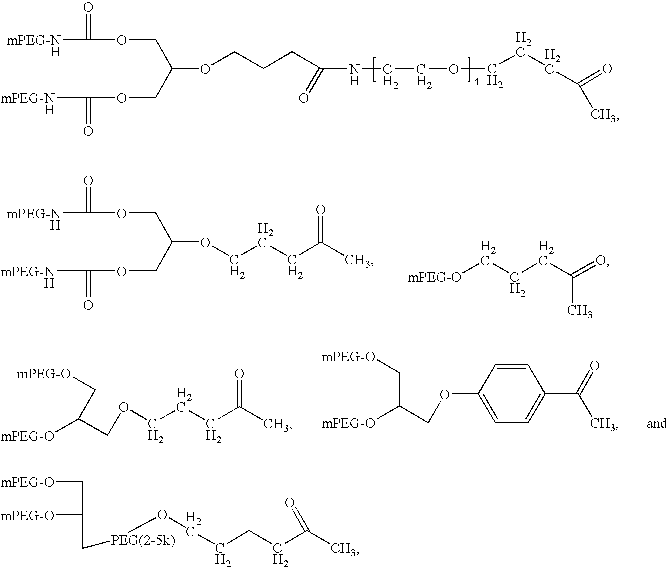

wherein, unless otherwise indicated, mPEG means mPEG with a molecular weight of 10 kDa, 20 kDa or 30 kDa and PEG means PEG with a molecular weight between 2 kDa and 5 kDa.

The compounds of formula [IV] are commercially available, e.g. from the companies Shearwater or NOF, or they may be readily obtained from commercially available compounds upon simple chemical modification.

The compounds of formula V may have improved or alternative pharmacological properties compared to the corresponding un-conjugated growth hormone, also referred to as the parent growth hormone. Hence, in one embodiment, the present invention relates to a method of modifying the pharmacological properties of growth hormone, the method comprising attaching PEG to said growth hormone according to the methods of the present invention. Examples of such pharmacological properties include functional in vivo half-life, immunogencity, renal filtration, protease protection and albumin binding.

The term “functional in vivo half-life” is used in its normal meaning, i.e., the time at which 50% of the biological activity of the growth hormone or conjugated growth hormone is still present in the body/target organ, or the time at which the activity of the growth hormone or growth hormone conjugate is 50% of its initial value. As an alternative to determining functional in vivo half-life, “in vivo plasma half-life” may be determined, i.e., the time at which 50% of the growth hormone or growth hormone conjugate circulate in the plasma or bloodstream prior to being cleared. Determination of plasma half-life is often more simple than determining functional half-life and the magnitude of plasma half-life is usually a good indication of the magnitude of functional in vivo half-life. Alternative terms to plasma half-life include serum half-life, circulating half-life, circulatory half-life, serum clearance, plasma clearance, and clearance half-life.

The term “increased” as used in connection with the functional in vivo half-life or plasma half-life is used to indicate that the relevant half-life of the growth hormone conjugate is statistically significantly increased relative to that of the parent growth hormone, as determined under comparable conditions. For instance the relevant half-life may be increased by at least about 25%, such as by at lest about 50%, e.g., by at least about 100%, 150%, 200%, 250%, or 500%. In one embodiment, the compounds of the present invention exhibit an increase in half-life of at least about 5 h, preferably at least about 24 h, more preferably at least about 72 h, and most preferably at least about 7 days, relative to the half-life of the parent GH.

Measurement of in vivo plasma half-life can be carried out in a number of ways as described in the literature. An increase in in vivo plasma half-life may be quantified as a decrease in clearance (CL) or as an increase in mean residence time (MRT). Conjugated growth hormone of the present invention for which the CL is decreased to less than 70%, such as less than 50%, such than less than 20%, such than less than 10% of the CL of the parent growth hormone as determined in a suitable assay is said to have an increased in vivo plasma half-life. Conjugated growth hormone of the present invention for which MRT is increased to more than 130%, such as more than 150%, such as more than 200%, such as more than 500% of the MRT of the parent growth hormone in a suitable assay is said to have an increased in vivo plasma half-life. Clearance and mean residence time can be assessed in standard pharmacokinetic studies using suitable test animals. It is within the capabilities of a person skilled in the art to choose a suitable test animal for a given protein. Tests in human, of course, represent the ultimate test. Suitable text animals include normal, Sprague-Dawley male rats, mice and cynomolgus monkeys. Typically the mice and rats are in injected in a single subcutaneous bolus, while monkeys may be injected in a single subcutaneous bolus or in a single iv dose. The amount injected depends on the test animal. Subsequently, blood samples are taken over a period of one to five days as appropriate for the assessment of CL and MRT. The blood samples are conveniently analysed by ELISA techniques.

The term “Immunogenicity” of a compound refers to the ability of the compound, when administered to a human, to elicit a deleterious immune response, whether humoral, cellular, or both. In any human sub-population, there may exist individuals who exhibit sensitivity to particular administered proteins. Immunogenicity may be measured by quantifying the presence of growth hormone antibodies and/or growth hormone responsive T-cells in a sensitive individual, using conventional methods known in the art. In one embodiment, the conjugated GH of the present invention exhibit a decrease in immunogenicity in a sensitive individual of at least about 10%, preferably at least about 25%, more preferably at least about 40% and most preferably at least about 50%, relative to the immunogenicity for that individual of the parent GH. In another aspect, immunogenicity may refer to the typical response in a population of similar subjects, such as the typical response in a patient population in a clinical trial.

The term “protease protection” or “protease protected” as used herein is intended to indicate that the conjugated growth hormone of the present invention is more resistant to the plasma peptidase or proteases than is the parent growth hormone. Protease and peptidase enzymes present in plasma are known to be involved in the degradation of circulating proteins, such as e.g. circulating peptide hormones, such as growth hormone.

Resistance of a protein to degradation by for instance dipeptidyl aminopeptidase IV (DPPIV) is determined by the following degradation assay: Aliquots of the protein (5 nmol) are incubated at 37° C. with 1 μL of purified dipeptidyl aminopeptidase IV corresponding to an enzymatic activity of 5 mU for 10-180 minutes in 100 μL of 0.1 M triethylamine-HCl buffer, pH 7.4. Enzymatic reactions are terminated by the addition of 5 μL of 10% trifluoroacetic acid, and the protein degradation products are separated and quantified using HPLC analysis. One method for performing this analysis is: The mixtures are applied onto a Vydac C18 widepore (30 nm pores, 5 μm particles) 250×4.6 mm column and eluted at a flow rate of 1 ml/min with linear stepwise gradients of acetonitrile in 0.1% trifluoroacetic acid (0% acetonitrile for 3 min, 0-24% acetonitrile for 17 min, 24-48% acetonitrile for 1 min) according to Siegel et al., Regul. Pept. 79, 93-102 (1999) and Mentlein et al. Eur. J. Biochem. 214, 829-35 (1993). Proteins and their degradation products may be monitored by their absorbance at 220 nm (peptide bonds) or 280 nm (aromatic amino acids), and are quantified by integration of their peak areas related to those of standards. The rate of hydrolysis of a protein by dipeptidyl aminopeptidase IV is estimated at incubation times which result in less than 10% of the peptide being hydrolysed. The resistance to other plasma proteases or peptidases may be determined in similar ways. In one embodiment, the rate of hydrolysis of the growth hormone conjugate is less than 70%, such as less than 40%, such as less than 10% of that of the parent growth hormone.

The most abundant protein component in circulating blood of mammalian species is serum albumin, which is normally present at a concentration of approximately 3 to 4.5 grams per 100 mL of whole blood. Serum albumin is a blood protein of approximately 70,000 daltons which has several important functions in the circulatory system. It functions as a transporter of a variety of organic molecules found in the blood, as the main transporter of various metabolites such as fatty acids and bilirubin through the blood, and, owing to its abundance, as an osmotic regulator of the circulating blood. Serum albumin has a half-life of more than one week, and one approach to increasing the plasma half-life of proteins has been to conjugate to the protein a group that binds to serum albumin. Albumin binding property may be determined as described in J. Med. Chem., 43, 1986-1992 (2000), which is incorporated herein by reference.

In one embodiment, the present invention relates to a compound according to formula [V]

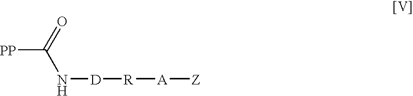

wherein PP represents a polypeptide radical obtained by removing —C(═O)—NH₂ from the side chain of a glutamine residue present in the polypeptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂— or C₅₋₁₅heteroalkylene;
A represents an oxime bond;
Z represents a moiety selected amongst

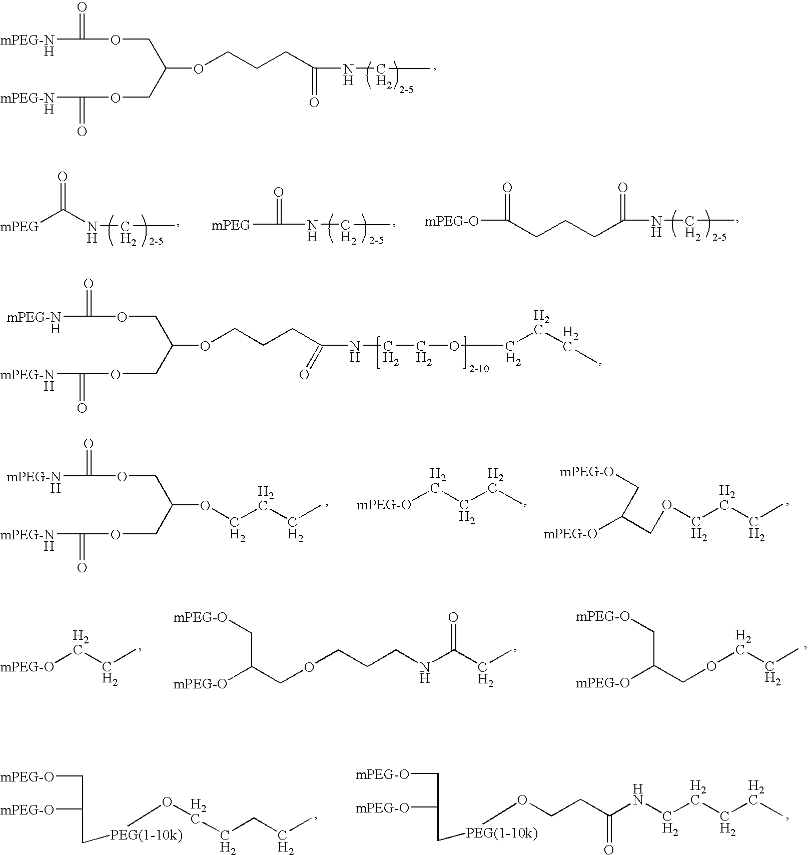

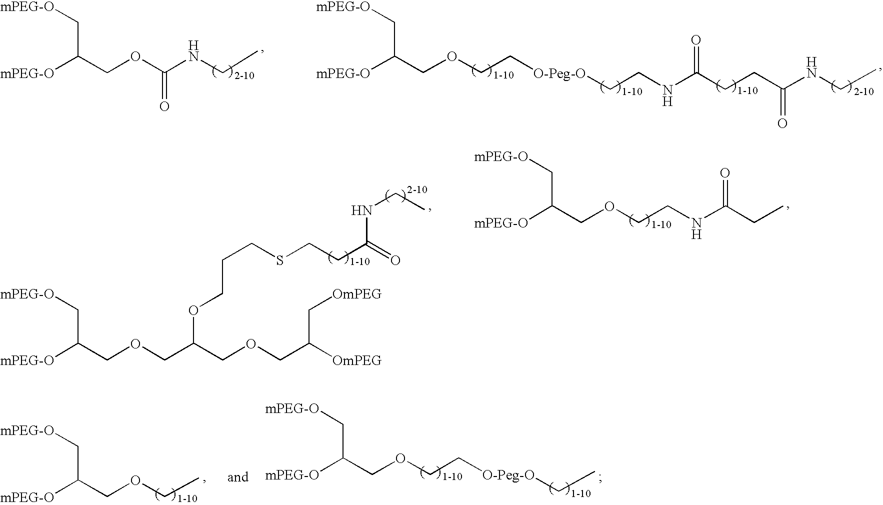

wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof.

In one embodiment, the polypeptide is a glutamine residue comprising growth hormone and PP represents a growth hormone radical obtained by removing —C(═O)—NH₂ from the side chain of a glutamine residue present in growth hormone.

In one embodiment, the invention relates to a growth hormone, such as hGH, which is covalently attached to one or more moieties comprising PEG, and in particular mPEG, wherein said PEG-comprising moiety is attached to the side chain of a glutamine residue present in the growth hormone. In one embodiment, the invention relates to hGH in which are covalently attached to a moiety comprising PEG, and in particular mPEG, wherein said PEG-comprising moiety is attached to the side chain of glutamine residue at the position corresponding to position 40, position 141 or position 40 and 141 in SEQ ID No. 1, provided it is not
N^{ε141}-[2-(4-(4-(mPEG(20k)ylbutanoyl)-amino-butyloxyimino)-ethyl]hGH,
N^{ε141}-[2-(1-(hexadecanoyl)piperidin-4-yl)ethyloxyimino)-ethyl]hGH,
N^{ε141}(2-(4-(4-(1,3-bis(mPEG(20k)ylaminocarbonyloxy)prop-2-yloxy)butyrylamino)butyloxyimino)ethyl)hGH,
N^{ε141}(2-(4-(2,6-bis(mPEG(20k)yloxycarbonylamino)hexanoylamino)butyloxyimino)ethyl)hGH,
N^{ε141}(2-(4-(4-(mPEG(30k)yloxy)butyrylamino)butyloxyimino)ethyl)hGH,
N^{ε141}(2-(4-(4-(mPEG(20k)yloxy)butyrylamino)butyloxyimino)ethyl)hGH, or
N^{ε141}(2-(4-(3-(mPEG(30k)yloxy)propanoylamino)butyloxyimino)ethyl)hGH.

In one embodiment, the invention relates to a compound of formula [Va]

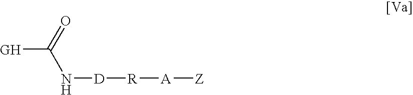

wherein GH represents a growth hormone radical obtained by removing —C(═O)—NH₂ from the side chain of a glutamine residue present in growth hormone;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂— or C₅₋₁₅heteroalkylene;
A represents an oxime bond;
Z represents a moiety selected amongst

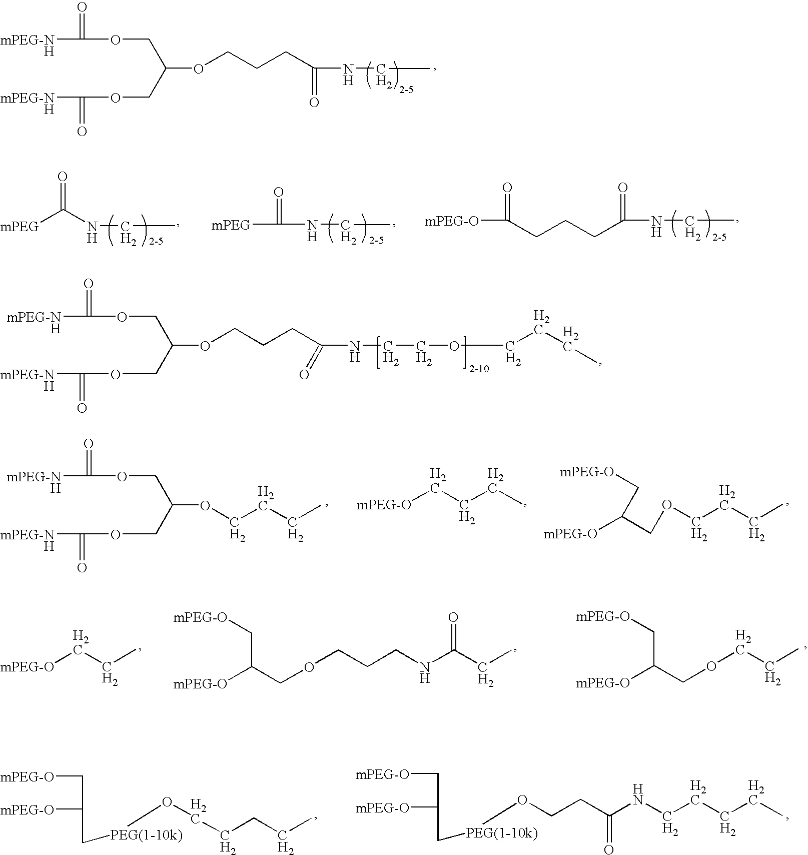

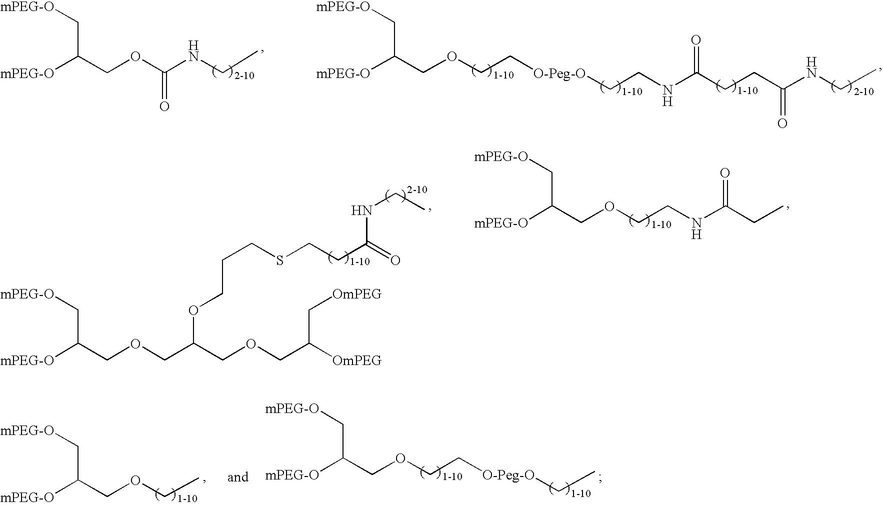

wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof;
provided that if Z is

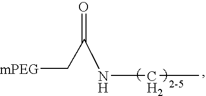

then mPEG is 10 kDa mPEG.

In one embodiment, D represents —O—.

In one embodiment, D represents a single bond.

In one embodiment, R represents —(CH₂)₄—CH(NH₂)—CO—NH—CH₂— or —(CH₂)₄—CH(NHCOCH₃)—CO—NH—CH₂—.

In one embodiment, R represents C₁₋₆alkylene. In a further embodiment, R represents C₁₋₃alkylene. In a further embodiment, R represents methylene, ethylene or propylene. In a further embodiment, R represents methylene or propylene.

In one embodiment, Z represents

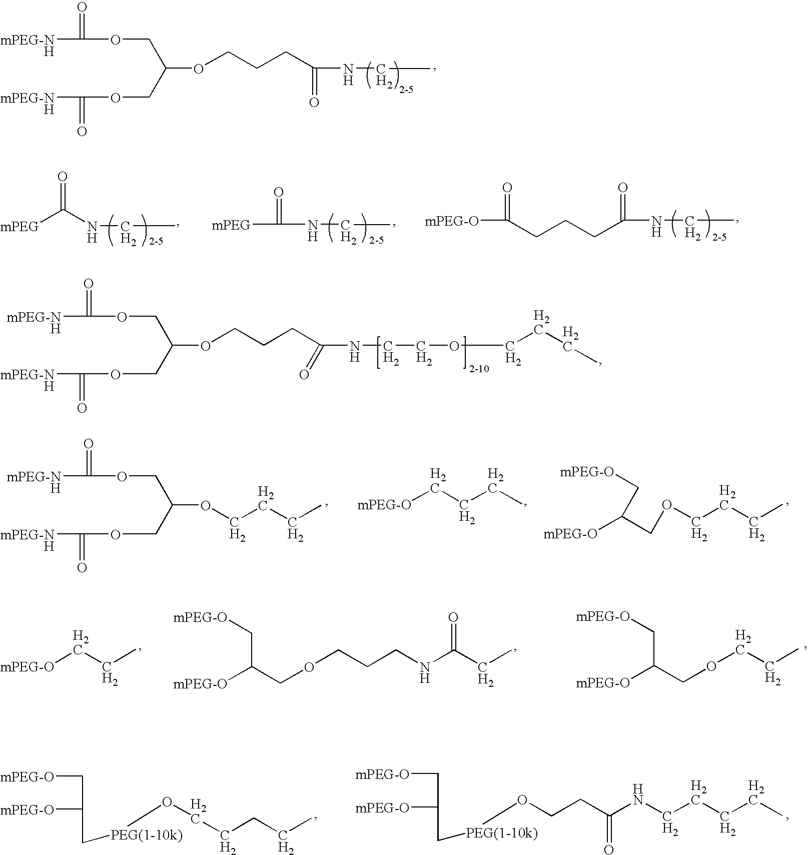

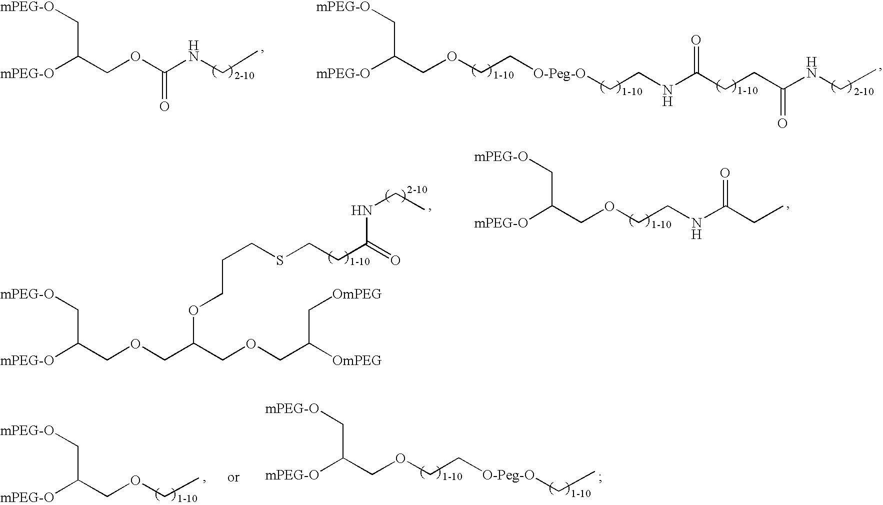

wherein, unless otherwise indicated, mPEG indicates a mPEG with a molecular weight of between 5 kDa and 40 kDa and PEG indicates a PEG with a molecular weight between 1 kDa and 10 kDa.

In one embodiment, GH represents the radical obtained by removing —C(═O)—NH₂ from the side chain of a glutamine residue present in an hGH comprising the amino acid sequence of SEQ ID No. 1.

In one embodiment, GH represents the radical obtained by removing —C(═O)—NH₂ from the side chain of a glutamine residue present in 20 kDa hGH.

In one embodiment, GH represents the radical obtained by removing —C(═O)—NH₂ from the side chain of glutamine 40 of hGH. In particular, said hGH may have been further modified by deleting glutamine 141 or substituting glutamine 141 with another amino acid, and in particular asparagine.

In one embodiment, GH represents the radical obtained by removing —C(═O)—NH₂ from the side chain of glutamine 141 of hGH. In particular, said hGH may have been further modified by deleting glutamine 40 or substituting glutamine 40 with another amino acid, and in particular asparagine.

In one embodiment, GH represents the radical obtained by removing —C(═O)—NH₂ from the side chain of a glutamine residue present in a position different from the position corresponding to position 40 in SEQ ID No. 1 and different from the position corresponding to position 141 in SEQ ID No. 1, wherein the glutamine residue in the position corresponding to position 40 in SEQ ID No. 1, and the glutamine residue in the position corresponding to position 141 in SEQ ID No. 1 each have been deleted or substituted with another amino acid, and in particular asparagine.

Particular examples of compounds of formula [Va] include
N^{δ141/40}-2-(O-(4-{4-(1,3-bis(mPEG(10k)ylaminocarbonyloxy)prop-2-yloxy)butyryl}aminobutyl)oximino)ethyl hGH;
N^{δ141/40}-2-(O-(4-{4-(mPEG(10k)yloxy)butyryl}aminobutyl)oximino)ethyl hGH;
N^{δ141/40}-2-(O-(4-{3-(mPEG(10k)yloxy)propionyl}aminobutyl)oximino)ethyl hGH;
N^{δ141/40}-2-(O-(4-{4-(2,3-bis(mPEG(10k)yloxy)prop-1-yloxy)butyryl}aminobutyl)oximino)ethyl hGH;
N^{δ141/40}-2-(O-(4-{5-(mPEG(10k)yloxy-5-oxopentanoyl}aminobutyl)oximino)ethyl hGH;
N^{δ141/40}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(10k)ylaminocarbonyloxy)prop-2-yloxy)butylidene}-aminoxy)propyloxy hGH;
N^{δ141/40}-3-({4-(mPEG(10k)yloxy)butylidene}aminoxy)propyloxy hGH;
N^{δ141/40}-3-({4-(2,3-bis(mPEG(10k)yloxy)prop-1-yloxy)butylidene}aminoxy)propyloxy hGH;
N^{δ141/40}-3-({3-(mPEG(10k)yloxy)propylidene}aminoxy)propyloxy hGH;
N^{δ141/40}-2-(O-2-(3-(2,3-bis(mPEG(10k)yloxy)propyloxy)propylamino)-2-oxoethyl)oximino)ethyl hGH;
N^{δ141/40}-3-({4-(2,3-bis(mPEG(10k)yloxy)prop-1-yloxy)propylidene}aminoxy)propyloxy hGH;
N^{δ141/40}-2-(O-(4-{4-(1,3-bis(mPEG(20k)ylaminocarbonyloxy)prop-2-yloxy)butyryl}aminobutyl)oximino)ethyl hGH;
N^{δ141/40}-2-(O-(4-{3-(mPEG(20k)yloxy)propionyl}aminobutyl)oximino)ethyl hGH;
N^{δ141/40}-2-(O-(4-{4-(2,3-bis(mPEG(20k)yloxy)prop-1-yloxy)butyryl}aminobutyl)oximino)ethyl hGH;
N^{δ141/40}-2-(O-(4-{5-(mPEG(20k)yloxy-5-oxopentanoyl}aminobutyl)oximino)ethyl hGH;
N^{δ141/40}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(20k)ylaminocarbonyloxy)prop-2-yloxy)butylidene}-aminoxy)propyloxy hGH;
N^{δ141/40}-3-({4-(mPEG(20k)yloxy)butylidene}aminoxy)propyloxy hGH;
N^{δ141/40}-3-({4-(2,3-bis(mPEG(20k)yloxy)prop-1-yloxy)butylidene}aminoxy)propyloxy hGH;
N^{δ141/40}-3-({3-(mPEG(20k)yloxy)propylidene}aminoxy)propyloxy hGH;
N^{δ141/40}-2-(O-(2-(3-(2,3-bis(mPEG(20k)yloxy)propyloxy)propylamino)-2-oxoethyl)oximino)ethyl hGH;
N^{δ141/40}-3-({4-(2,3-bis(mPEG(20k)yloxy)prop-1-yloxy)propylidene}aminoxy)propyloxy hGH;
N^{δ141/40}-2-(O-(4-{4-(1,3-bis(mPEG(30k)ylaminocarbonyloxy)prop-2-yloxy)butyryl}aminobutyl)oximino)ethyl hGH;
N^{δ141/40}-2-(O-(4-{3-(mPEG(30k)yloxy)propionyl}aminobutyl)oximino)ethyl hGH;
N^{δ141/40}-2-(O-(4-{4-(2,3-bis(mPEG(30k)yloxy)prop-1-yloxy)butyryl}aminobutyl)oximino)ethyl hGH;
N^{δ141/40}-2-(O-(4-{5-(mPEG(30k)yloxy-5-oxopentanoyl}aminobutyl)oximino)ethyl hGH;
N^{δ141/40}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(30k)ylaminocarbonyloxy)prop-2-yloxy)butylidene}-aminoxy)propyloxy hGH;
N^{δ141/40}-3-({4-(mPEG(30k)yloxy)butylidene}aminoxy)propyloxy hGH;
N^{δ141/40}-3-({4-(2,3-bis(mPEG(30k)yloxy)prop-1-yloxy)butylidene}aminoxy)propyloxy hGH;
N^{δ141/40}-3-({3-(mPEG(30k)yloxy)propylidene}aminoxy)propyloxy hGH;
N^{δ141/40}-2-(O-(2-(3-(2,3-bis(mPEG(30k)yloxy)propyloxy)propylamino)-2-oxoethyl)oximino)ethyl hGH;
N^{δ141/40}-3-({4-(2,3-bis(mPEG(30k)yloxy)prop-1-yloxy)propylidene}aminoxy)propyloxy hGH;
N^{δ141/40}-3-({4-{(2,3-bis(mPEG(20k)yl)prop-1-yloxy)PEGyloxy}butylidene}aminoxy)propyloxy hGH;
N^{δ141/40}-2-((4-(4-((2,3-bis(mPEG(20k)yl)propyl)PEGyloxy)butyrylamino)butyl)oximino)ethyl hGH;
N^{δ141}-2-(O-(4-{4-(1,3-bis(mPEG(10k)ylaminocarbonyloxy)prop-2-yloxy)butyryl}-aminobutyl)oximino)ethyl hGH;
N^{δ141}-2-(O-(4-{4-(mPEG(10k)yloxy)butyryl}aminobutyl)oximino)ethyl hGH;
N^{δ141}-2-(O-(4-{3-(mPEG(10k)yloxy)propionyl}aminobutyl)oximino)ethyl hGH;
N^{δ141}-2-(O-(4-{4-(2,3-bis(mPEG(10k)yloxy)prop-1-yloxy)butyryl}aminobutyl)oximino)ethyl hGH;
N^{δ141}-2-(O-(4-{5-(mPEG(10k)yloxy-5-oxopentanoyl}aminobutyl)oximino)ethyl hGH;
N^{δ141}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(10k)ylaminocarbonyloxy)prop-2-yloxy)butylidene}-aminoxy)propyloxy hGH;
N^{δ141}-3-({4-(mPEG(10k)yloxy)butylidene}aminoxy)propyloxy hGH;
N^{δ141}-3-({4-(2,3-bis(mPEG(10k)yloxy)prop-1-yloxy)butylidene}aminoxy)propyloxy hGH;
N^{δ141}-3-({3-(mPEG(10k)yloxy)propylidene}aminoxy)propyloxy hGH;
N^{δ141}-2-(O-(2-(3-(2,3-bis(mPEG(10k)yloxy)propyloxy)propylamino)-2-oxoethyl)oximino)ethyl hGH,
N^{δ141}-3-({4-(2,3-bis(mPEG(10k)yloxy)prop-1-yloxy)propylidene}aminoxy)propyloxy hGH;
N^{δ141}-2-(O-(4-{4-(1,3-bis(mPEG(20k)ylaminocarbonyloxy)prop-2-yloxy)butyryl}-minobutyl)oximino)ethyl hGH;
N^{δ141}-2-(O-(4-{3-(mPEG(20k)yloxy)propionyl}aminobutyl)oximino)ethyl hGH;
N^{δ141}-2-(O-(4-{4-(2,3-bis(mPEG(20k)yloxy)prop-1-yloxy)butyryl}aminobutyl)oximino)ethyl hGH;
N^{δ141}-2-(O-(4-{5-(mPEG(20k)yloxy-5-oxopentanoyl}aminobutyl)oximino)ethyl hGH;
N^{δ141}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(20k)ylaminocarbonyloxy)prop-2-yloxy)butylidene}-aminoxy)propyloxy hGH;
N^{δ141}-3-({4-(mPEG(20k)yloxy)butylidene}aminoxy)propyloxy hGH;
N^{δ141}-3-({4-(2,3-bis(mPEG(20k)yloxy)prop-1-yloxy)butylidene}aminoxy)propyloxy hGH;
N^{δ141}-3-({3-(mPEG(20k)yloxy)propylidene}aminoxy)propyloxy hGH;
N^{δ141}-2-(O-(2-(3-(2,3-bis(mPEG(20k)yloxy)propyloxy)propylamino)-2-oxoethyl)oximino)ethyl hGH;
N^{δ141}-3-({4-(2,3-bis(mPEG(20k)yloxy)prop-1-yloxy)propylidene}aminoxy)propyloxy hGH;
N^{δ141}-2-(O-(4-{4-(1,3-bis(mPEG(30k)ylaminocarbonyloxy)prop-2-yloxy)butyryl}-aminobutyl)oximino)ethyl hGH;
N^{δ141}-2-(O-(4-{3-(mPEG(30k)yloxy)propionyl}aminobutyl)oximino)ethyl hGH;
N^{δ141}-2-(O-(4-{4-(2,3-bis(mPEG(30k)yloxy)prop-1-yloxy)butyryl}aminobutyl)oximino)ethyl hGH,
N^{δ141}-2-(O-(4-{5-(mPEG(30k)yloxy-5-oxopentanoyl}aminobutyl)oximino)ethyl hGH;
N^{δ141}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(30k)ylaminocarbonyloxy)prop-2-yloxy)butylidene}-aminoxy)propyloxy hGH;
N^{δ141}-3-({4-(mPEG(30k)yloxy)butylidene}aminoxy)propyloxy hGH;
N^{δ141}-3-({4-(2,3-bis(mPEG(30k)yloxy)prop-1-yloxy)butylidene}aminoxy)propyloxy hGH;
N^{δ141}-3-({3-(mPEG(30k)yloxy)propylidene}aminoxy)propyloxy hGH;
N^{δ141}-2-(O-(2-(3-(2,3-bis(mPEG(30k)yloxy)propyloxy)propylamino)-2-oxoethyl)oximino)ethyl hGH;
N^{δ141}-3-({4-(2,3-bis(mPEG(30k)yloxy)prop-1-yloxy)propylidene}aminoxy)propyloxy hGH;
N^{δ141}-3-({4-{(2,3-bis(mPEG(20k)yloxy)prop-1-yl)PEGyloxy}butylidene}aminoxy)propyloxy hGH;
N^{δ141}-2-((4-(4-((2,3-bis(mPEG(20k)yl)propyl)PEGyloxy)butyrylamino)butyl)oximino)ethyl hGH;
N^{δ40}-2-(O-(4-{4-(1,3-bis(mPEG(10k)ylaminocarbonyloxy)prop-2-yloxy)butyryl}-aminobutyl)oximino)ethyl hGH;
N^{δ40}-2-(O-(4-{4-(mPEG(10k)yloxy)butyryl}aminobutyl)oximino)ethyl hGH;
N^{δ40}-2-(O-(4-{3-(mPEG(10k)yloxy)propionyl}aminobutyl)oximino)ethyl hGH;
N^{δ40}-2-(O-(4-{4-(2,3-bis(mPEG(10k)yloxy)prop-1-yloxy)butyryl}aminobutyl)oximino)ethyl hGH;
N^{δ40}-2-(O-(4-{5-(mPEG(10k)yloxy-5-oxopentanoyl}aminobutyl)oximino)ethyl hGH;
N^{δ40}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(10k)ylaminocarbonyloxy)prop-2-yloxy)butylidene}-aminoxy)propyloxy hGH;
N^{δ40}-3-({4-(mPEG(10k)yloxy)butylidene}aminoxy)propyloxy hGH;
N^{δ40}-3-({4-(2,3-bis(mPEG(10k)yloxy)prop-1-yloxy)butylidene}aminoxy)propyloxy hGH;
N^{δ40}-3-({3-(mPEG(10k)yloxy)propylidene}aminoxy)propyloxy hGH;
N^{δ40}-2-(O-(2-(3-(2,3-bis(mPEG(10k)yloxy)propyloxy)propylamino)-2-oxoethyl)oximino)ethyl hGH,
N^{δ40}-3-({4-(2,3-bis(mPEG(10k)yloxy)prop-1-yloxy)propylidene}aminoxy)propyloxy hGH;
N^{δ40}-2-(O-(4-{4-(1,3-bis(mPEG(20k)ylaminocarbonyloxy)prop-2-yloxy)butyryl}-aminobutyl)oximino)ethyl hGH;
N^{δ40}-2-(O-(4-{3-(mPEG(20k)yloxy)propionyl}aminobutyl)oximino)ethyl hGH;
N^{δ40}-2-(O-(4-{4-(2,3-bis(mPEG(20k)yloxy)prop-1-yloxy)butyryl}aminobutyl)oximino)ethyl hGH;
N^{δ40}-2-(O-(4-{5-(mPEG(20k)yloxy-5-oxopentanoyl}aminobutyl)oximino)ethyl hGH;
N^{δ40}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(20k)ylaminocarbonyloxy)prop-2-yloxy)butylidene}-aminoxy)propyloxy hGH;
N^{δ40}-3-({4-(mPEG(20k)yloxy)butylidene}aminoxy)propyloxy hGH;
N^{δ40}-3-({4-(2,3-bis(mPEG(20k)yloxy)prop-1-yloxy)butylidene}aminoxy)propyloxy hGH;
N^{δ40}-3-({3-(mPEG(20k)yloxy)propylidene}aminoxy)propyloxy hGH;
N^{δ40}-2-(O-(2-(3-(2,3-bis(mPEG(20k)yloxy)propyloxy)propylamino)-2-oxoethyl)oximino)ethyl hGH,
N^{δ40}-3-({4-(2,3-bis(mPEG(20k)yloxy)prop-1-yloxy)propylidene}aminoxy)propyloxy hGH;
N^{δ40}-2-(O-(4-{4-(1,3-bis(mPEG(30k)ylaminocarbonyloxy)prop-2-yloxy)butyryl}-aminobutyl)-oximino)ethyl hGH;
N^{δ40}-2-(O-(4-{3-(mPEG(30k)yloxy)propionyl}aminobutyl)oximino)ethyl hGH;
N^{δ40}-2-(O-(4-{4-(2,3-bis(mPEG(30k)yloxy)prop-1-yloxy)butyryl}aminobutyl)oximino)ethyl hGH;
N^{δ40}-2-(O-(4-{5-(mPEG(30k)yloxy-5-oxopentanoyl}aminobutyl)oximino)ethyl hGH;
N^{δ40}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(30k)ylaminocarbonyloxy)prop-2-yloxy)butylidene}-aminoxy)propyloxy hGH;
N^{δ40}-3-({4-(mPEG(30k)yloxy)butylidene}aminoxy)propyloxy hGH;
N^{δ40}-3-({4-(2,3-bis(mPEG(30k)yloxy)prop-1-yloxy)butylidene}aminoxy)propyloxy hGH;
N^{δ40}-3-({3-(mPEG(30k)yloxy)propylidene}aminoxy)propyloxy hGH;
N^{δ40}-2-(O-(2-(3-(2,3-bis(mPEG(30k)yloxy)propyloxy)propylamino)-2-oxoethyl)oximino)ethyl hGH,
N^{δ40}-3-({4-(2,3-bis(mPEG(30k)yloxy)prop-1-yloxy)propylidene}aminoxy)propyloxy hGH;
N^{δ40}-3-({4-{(2,3-bis(mPEG(20k)yloxy)prop-1-yl)PEGyloxy}butylidene}aminoxy)propyloxy hGH;
N^{δ40}-2-((4-(4-((2,3-bis(mPEG(20k)yl)propyl)PEGyloxy)butyrylamino)butyl)oximino)ethyl hGH;
N^{ε}141-[2-O-(4-(4-(1,3-bis(mPEG(20K)aminocarbonyloxy)-2-propyloxy)butyrylamino)butyl)oxyimino)ethyl]hGH;
N^{ε}141-[2-(O-(4-(4-(mPEG(30K)oxy)butyrylamino)-butyl)oxyimino)-ethyl]hGH;
N^{ε}141-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}141-[2-(2,3-(mPEG(20K)yloxy)propyloxycarbonylamino)ethyloximino)ethyl]hGH;
N^{ε}141-[2-(O-(2-(2-(mPEG(40K)yloxy)ethylamino)-2-oxoethyl)oximino)ethyl]hGH;
N^{ε}141-[2-(3-(4-((1,3-bis(mPEG(30K)ylaminocarbonyloxy)-2-propyloxy)butylidene)aminoxy)propyloxyimino)ethyl]hGH;
N^{ε}141/40-[2-O-(4-(4-(1,3-bis(mPEG(20K)aminocarbonyloxy)-2-propyloxy)butyrylamino)butyl)oxyimino)ethyl]hGH;
N^{ε}141/40-[2-(O-(4-(4-(mPEG(30K)oxy)butyrylamino)-butyl)oxyimino)-ethyl]hGH;
N^{ε}141/40-(3-((4-(2-(2-(2-(2-(4-(1,3-bis(m PEG(20K)ylaminocarbonyloxy)-2-propyloxy)butyrylamino)ethoxy)ethoxy)ethoxy)ethoxy)butylidene)aminoxy)propyloxy)hGH;
N^{ε}141/40-[2-(2-(2,3-(mPEG(20K)yloxy)propyloxycarbonylamino)ethyloximino)ethyl]hGH;
N^{ε}141/40-[2-(O-(2-(2-(mPEG(40K)yloxy)ethylamino)-2-oxoethyl)oximino)ethyl]hGH;
N^{ε}141/40-[2-(3-(4-((1,3-bis(mPEG(30K)ylaminocarbonyloxy)-2-propyloxy)butylidene)aminoxy)propyloxyimino)ethyl]hGH;
N^{ε}141/40-[2-O-(4-(4-(1,3-bis(mPEG(20K)aminocarbonyloxy)-2-propyloxy)butyrylamino)butyl)oxyimino)ethyl]hGH;
N^{ε}40-[2-(O-(4-(4-(mPEG(30K)oxy)butyrylamino)-butyl)oxyimino)-ethyl]hGH;
N^{ε}40-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}40-[2-(2-(2,3-(mPEG(20K)yloxy)propyloxycarbonylamino)ethyloximino)ethyl]hGH;
N^{ε}40-[2-(O-(2-(2-(mPEG(40K)yloxy)ethylamino)-2-oxoethyl)oximino)ethyl]hGH;
N^{ε}40-[2-(3-(4-((1,3-bis(mPEG(30K)ylaminocarbonyloxy)-2-propyloxy)butylidene)aminoxy)propyloxyimino)ethyl]hGH;

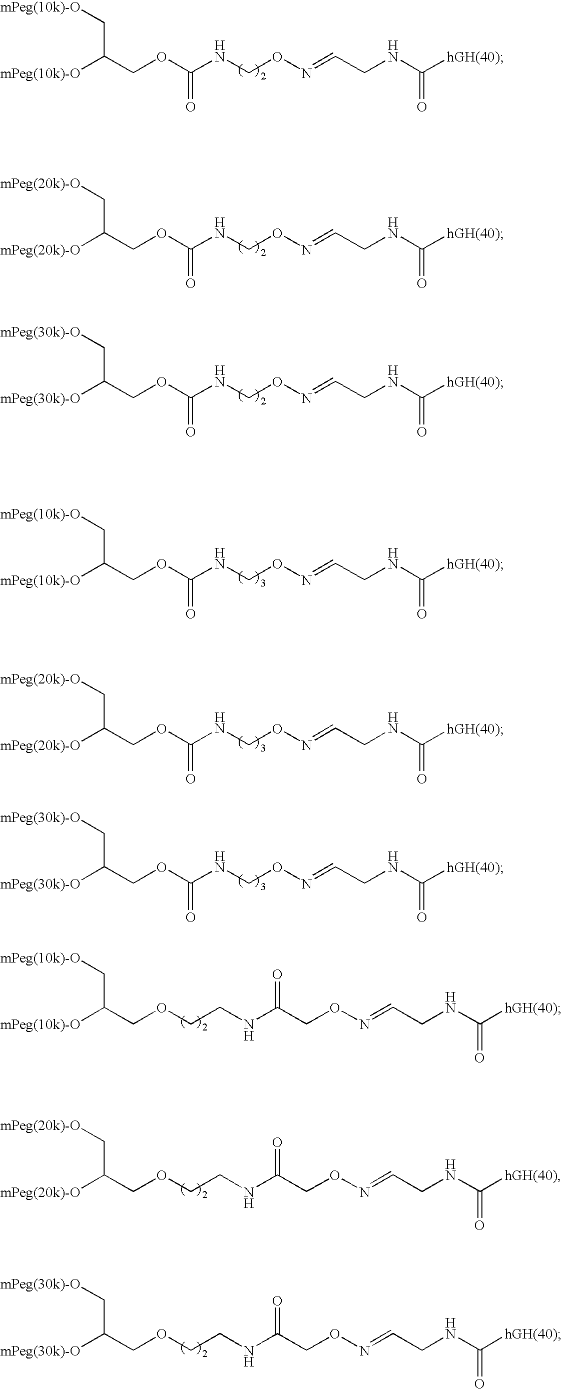

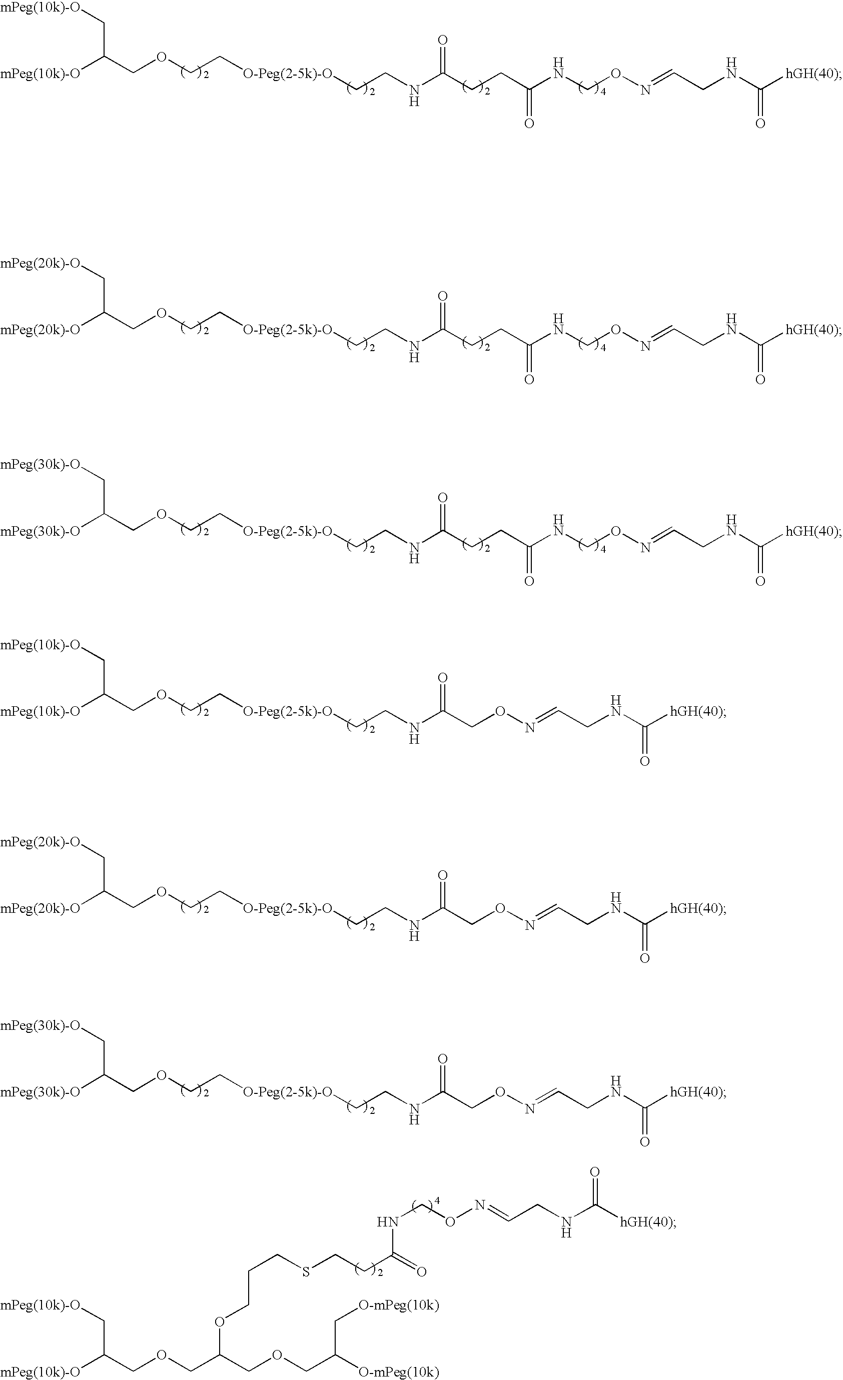

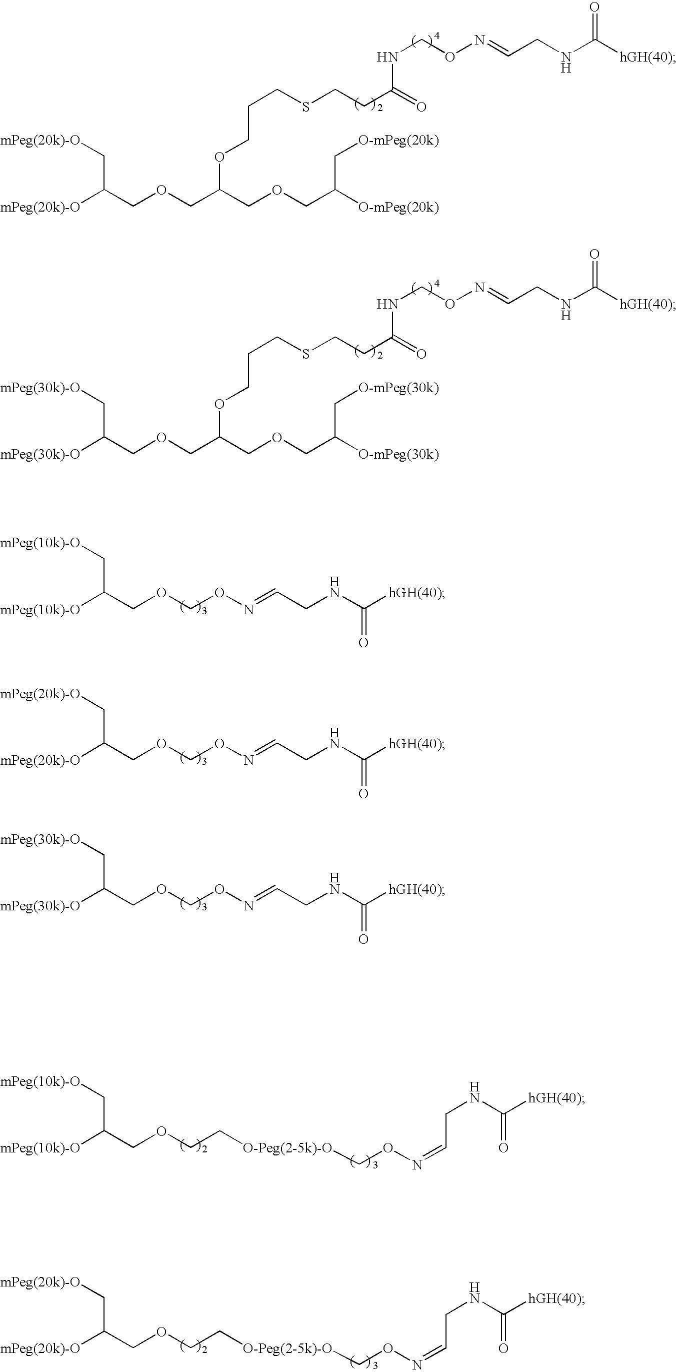

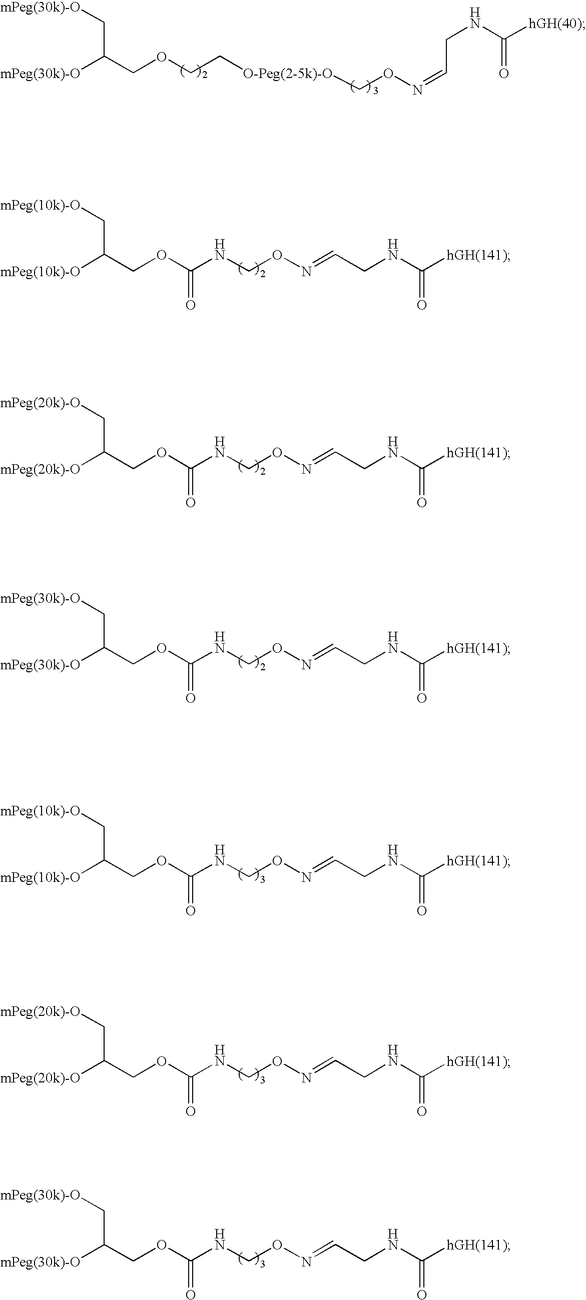

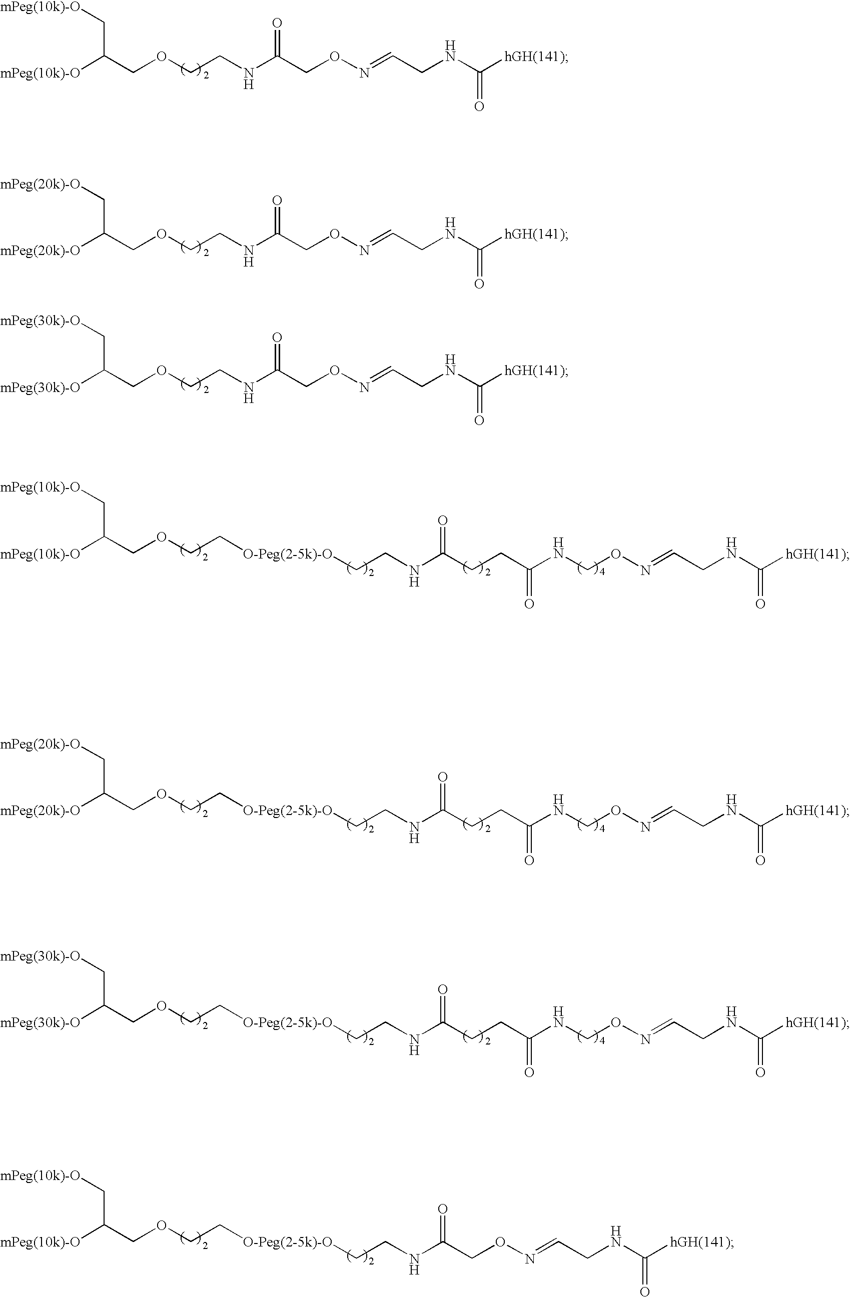

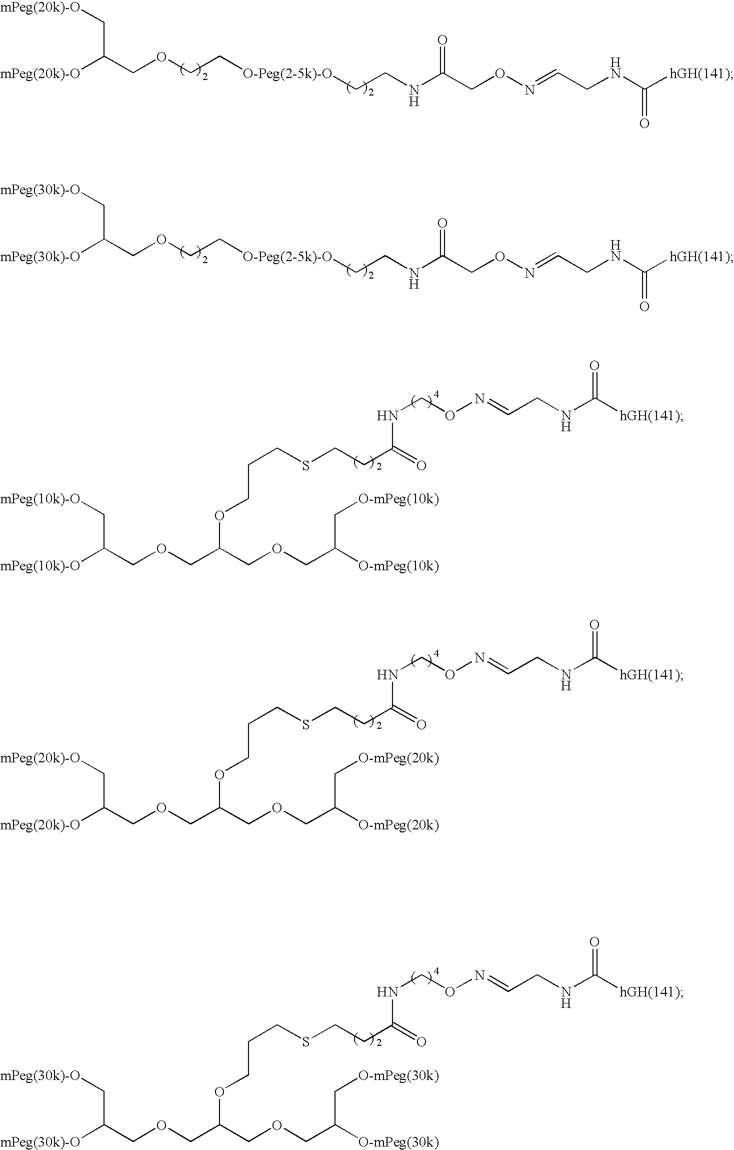

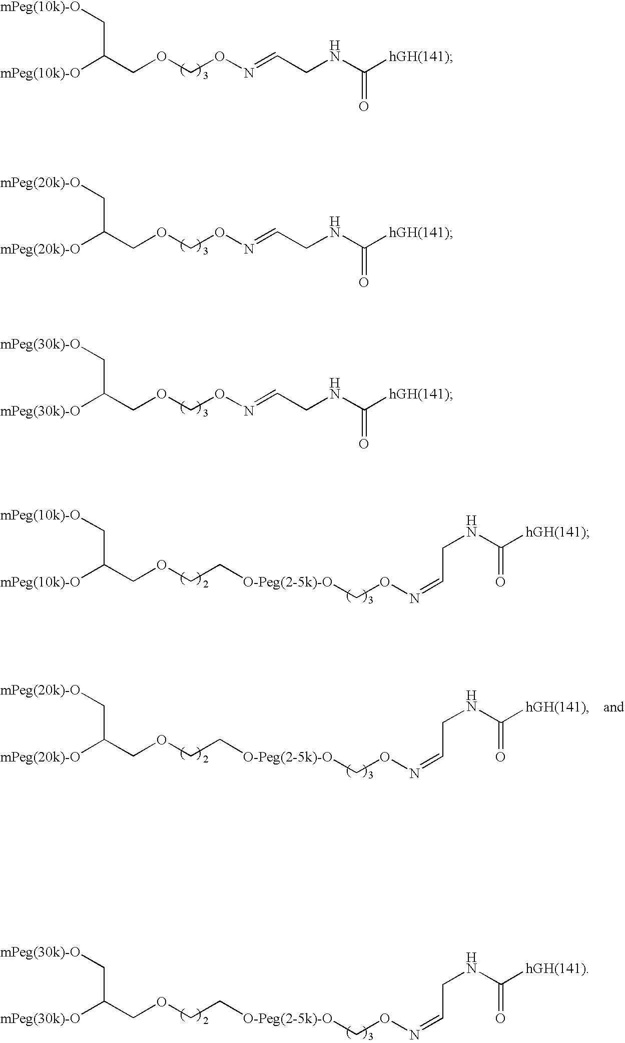

Some of the above graphical representations may also or alternatively be represented as
N^{δ141}-(2-(O-(2-(2,3-Bis(mPEG(10k)yloxy)propyloxycarbonylamino)ethyl)oximino)ethyl)hGH,
N^{δ141}-(2-(O-(3-(2,3-Bis(mPEG(10k)yloxy)propyloxycarbonylamino)propyl)oximino)ethyl)hGH,
N^{δ40}-(2-(O-(2-(2,3-Bis(mPEG(10k)yloxy)propyloxycarbonylamino)ethyl)oximino)ethyl)hGH,
N^{δ40}-(2-(O-(3-(2,3-Bis(mPEG(10k)yloxy)propyloxycarbonylamino)propyl)oximino)ethyl)hGH,
N^{δ141}-(2-O-(2-oxo-2-(3-(2,3-Bis(mPEG(10k)yloxy)propyloxy)propylamino)ethyl)oximinoethyl)hGH,
N^{δ40}-(2-O-(2-oxo-2-(3-(2,3-Bis(mPEG(10k)yloxy)propyloxy)propylamino)ethyl)oximinoethyl)hGH,
N^{δ141}-(2-O-(4-(5-(3-(omega-(3-(2,3-bis(mPEG(10k)yloxy)propyloxy)propyloxy)polyoxyethylenyloxy)propylamino)-1,5-dioxopentylamino)butyl)oximinoethyl)hGH,
N^{δ141}-(2-(O-(2-(2,3-Bis(mPEG(20k)yloxy)propyloxycarbonylamino)ethyl)oximino)ethyl)hGH
N^{δ40}-(2-(O-(3-(2,3-Bis(mPEG(20k)yloxy)propyloxycarbonylamino)propyl)oximino)ethyl)hGH
N^{δ141}-(2-(O-(2-(2,3-Bis(mPEG(20k)yloxy)propyloxycarbonylamino)ethyl)oximino)ethyl)hGH
N^{δ40}-(2-(O-(3-(2,3-Bis(mPEG(20k)yloxy)propyloxycarbonylamino)propyl)oximino)ethyl)hGH
N^{δ141}-(2-O-(2-oxo-2-(3-(2,3-Bis(mPEG(20k)yloxy)propyloxy)propylamino)ethyl)oximinoethyl)hGH
N^{δ40}-(2-O-(2-oxo-2-(3-(2,3-Bis(mPEG(20k)yloxy)propyloxy)propylamino)ethyl)oximinoethyl)hGH,
N^{δ141}-(2-O-(4-(5-(3-(omega-(3-(2,3-bis(mPEG(20k)yloxy)propyloxy)propyloxy)polyoxyethylenyloxy)propylamino)-1,5-dioxopentylamino)butyl)oximinoethyl)hGH,
N^{δ141}-(2-(O-(2-(2,3-Bis(mPEG(30k)yloxy)propyloxycarbonylamino)ethyl)oximino)ethyl)hGH,
N^{δ141}-(2-(O-(3-(2,3-Bis(mPEG(30k)yloxy)propyloxycarbonylamino)propyl)oximino)ethyl)hGH,
N^{δ40}-(2-(O-(2-(2,3-Bis(mPEG(30k)yloxy)propyloxycarbonylamino)ethyl)oximino)ethyl)hGH,
N^{δ40}-(2-(O-(3-(2,3-Bis(mPEG(30k)yloxy)propyloxycarbonylamino)propyl)oximino)ethyl)hGH,
N^{δ141}-(2-O-(2-oxo-2-(3-(2,3-Bis(mPEG(30k)yloxy)propyloxy)propylamino)ethyl)oximinoethyl)hGH,
N^{δ40}-(2-O-(2-oxo-2-(3-(2,3-Bis(mPEG(30k)yloxy)propyloxy)propylamino)ethyl)oximinoethyl)hGH, and
N^{δ141}-(2-O-(4-(5-(3-(omega-(3-(2,3-bis(mPEG(30k)yloxy)propyloxy)propyloxy)polyoxyethylenyloxy)propylamino)-1,5-dioxopentylamino)butyl)oximinoethyl)hGH.

In one embodiment, the present invention relates to compound according to formula [VI]

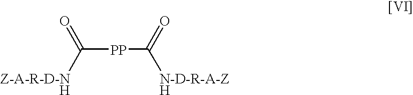

wherein PP represents a polypeptide radical obtained by removing —C(═O)—NH₂ from the side chain of two glutamine residues present in said polypeptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
A represents an oxime bond;
Z represents a moiety selected amongst

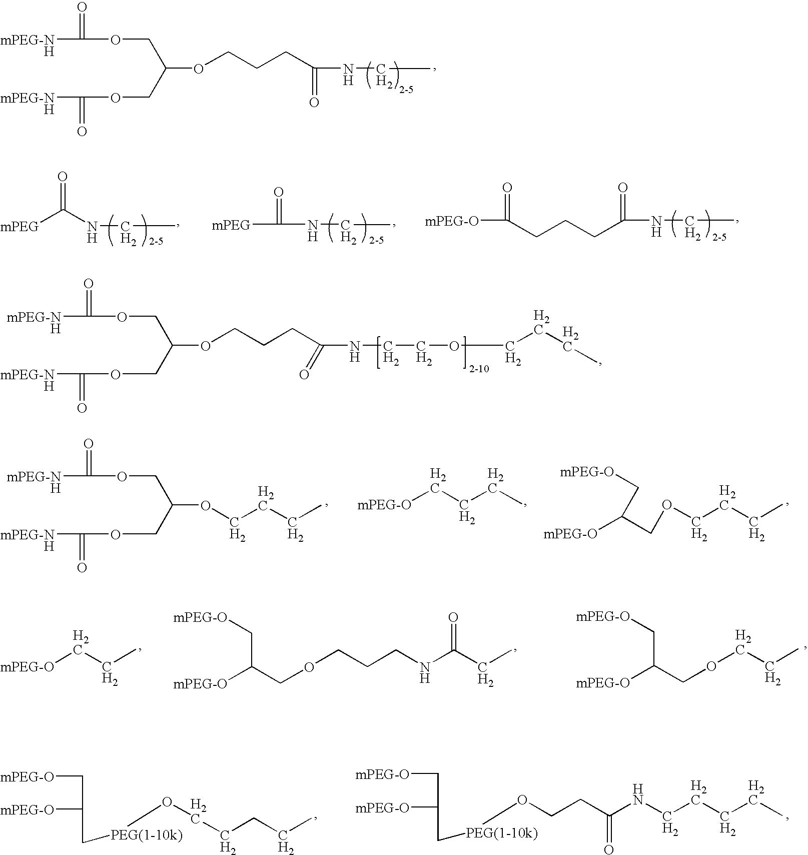

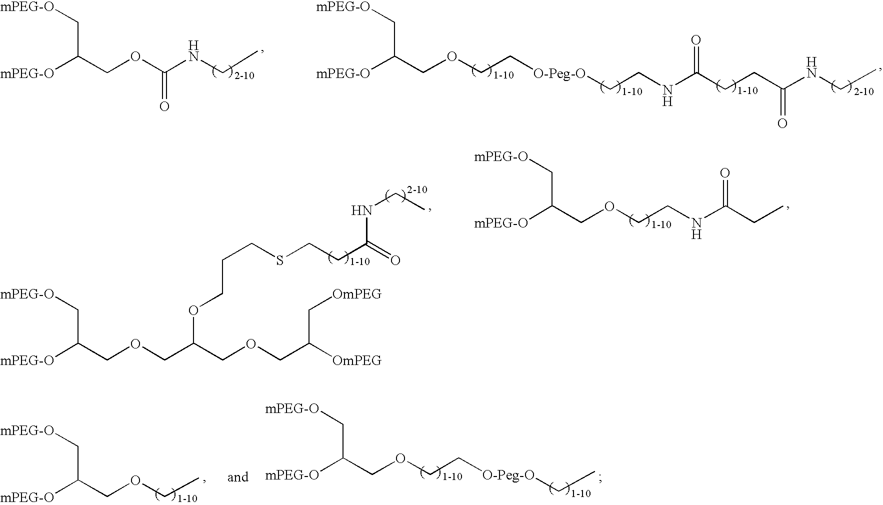

wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof.

In one embodiment, the polypeptide is a glutamine residue comprising growth hormone and PP represents a growth hormone radical obtained by removing —C(═O)—NH₂ from the side chain of two glutamine residues present in the growth hormone.

In one embodiment, the present invention provides a compound according to formula [VIa]

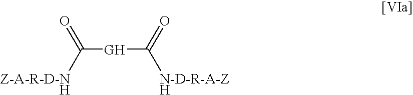

wherein GH represents a growth hormone radical obtained by removing —C(═O)—NH₂ from the side chain of two glutamine residues present in said growth hormone;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
A represents an oxime bond;
Z represents a moiety selected amongst

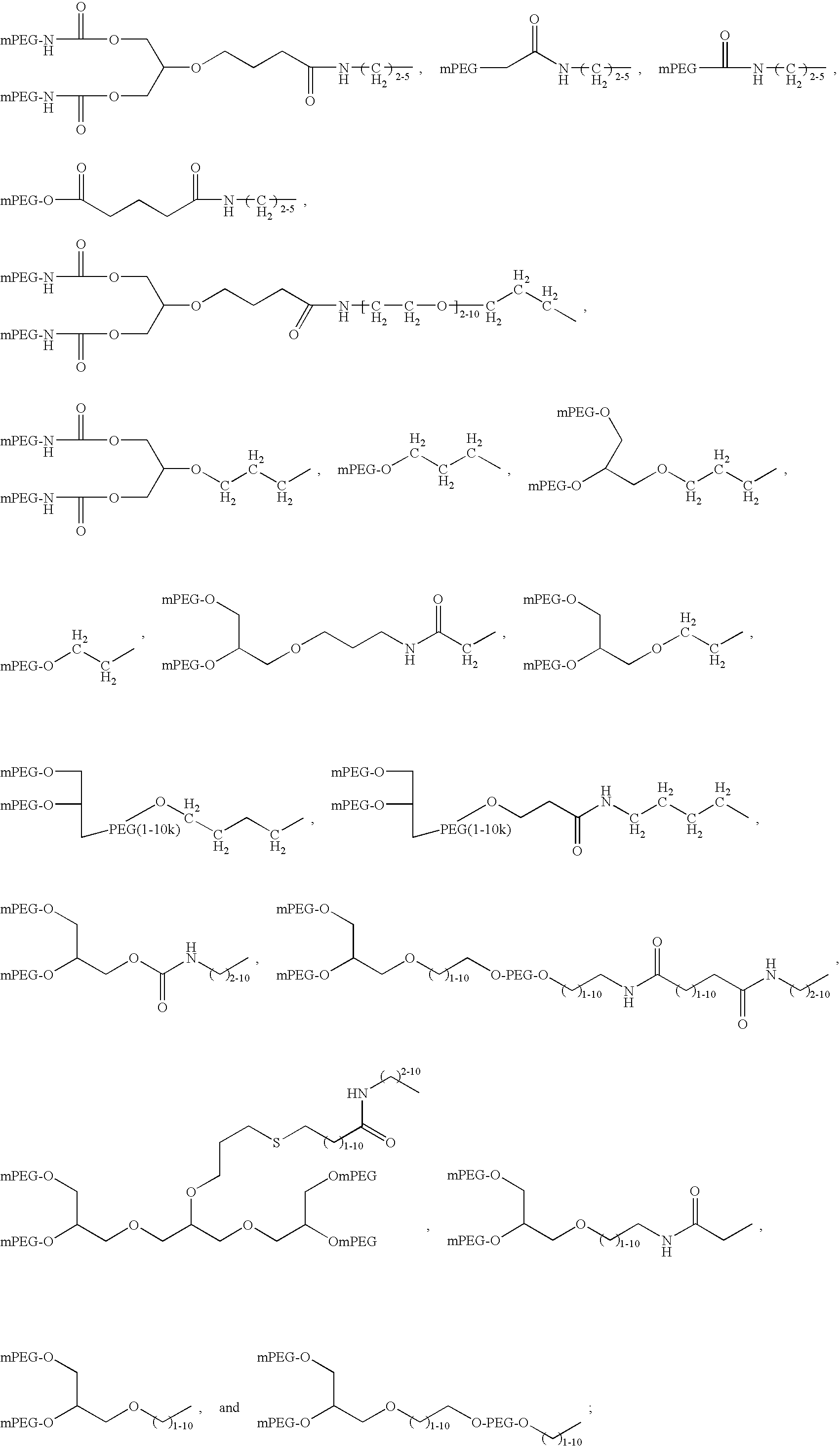

wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof.

In one embodiment, D represents —O—.

In one embodiment, D represents a single bond.

In one embodiment, R represents —(CH₂)₄—CH(NH₂)—CO—NH—CH₂— or —(CH₂)₄—CH(NHCOCH₃)—CO—NH—CH₂—.

In one embodiment, R represents C₁₋₆alkylene. In a further embodiment, R represents C₁₋₃alkylene. In a further embodiment, R represents methylene, ethylene or propylene. In a further embodiment, R represents methylene or propylene.

In one embodiment, Z represents

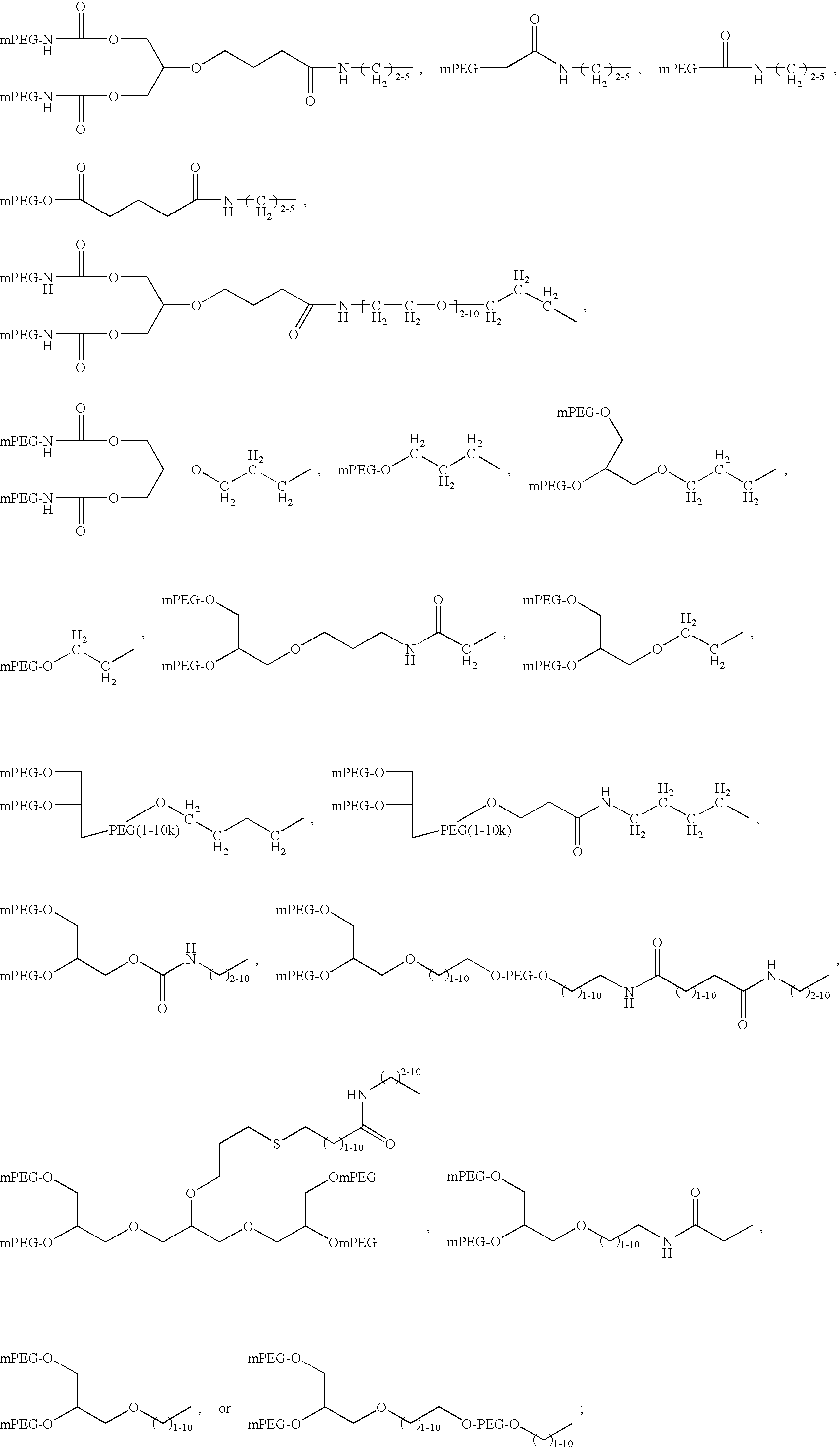

wherein, unless otherwise indicated, mPEG indicates a mPEG with a molecular weight of between 5 kDa and 40 kDa and PEG indicates a PEG with a molecular weight between 1 kDa and 10 kDa.

In one embodiment, GH in formula [VIa] represents the radical obtained by removing —C(═O)—NH₂ from the side chain of two glutamine residues present in an hGH comprising the amino acid sequence of SEQ ID No. 1.

In one embodiment, GH in formula [VIa] represents the radical obtained by removing —C(═O)—NH₂ from the side chain of two glutamine residues present in 20 kDa hGH.

In one embodiment, GH in formula [VIa] represents the radical obtained by removing —C(═O)—NH₂ from the side chain of a glutamine residue in the position corresponding to position 40 in SEQ ID No. 1, and from the side chain of the glutamine residue in the position corresponding to position 141 in SEQ ID No. 1; or

represents the radical obtained by removing —C(═O)—NH₂ from the side chain of a glutamine residue in the position corresponding to position 40 in SEQ ID No. 1, wherein the glutamine residue in the position corresponding to position 141 in SEQ ID No. 1 has been deleted or substituted with another amino acid, and by removing —C(═O)—NH₂ from the side chain of another glutamine residue present in a position different from the positions corresponding to positions 40 and 141 in SEQ ID No. 1; or
represents the radical obtained by removing —C(═O)—NH₂ from the side chain of a glutamine residue in the position corresponding to position 141 in SEQ ID No. 1, wherein the glutamine residue in the position corresponding to position 40 in SEQ ID No. 1 has been deleted or substituted with another amino acid, and by removing —C(═O)—NH₂ from the side chain of another glutamine residue present in a position different from the positions corresponding to positions 40 and 141 in SEQ ID No. 1; or
represents the radical obtained by removing —C(═O)—NH₂ from the side chain of two glutamines present in an hGH in positions different from the positions corresponding to positions 40 and 141 in SEQ ID No. 1, wherein any glutamine residues present in the positions corresponding to positions 40 and 141 in SEQ ID No. 1 has been deleted or substituted with other amino acids.

In an additional or alternative embodiment, the invention provides the methods and compounds described herein, wherein each instance of “mPEG” is replaced by an alkoxyPEG or “aPEG” compound of the formula

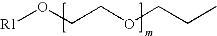

wherein m is an integer larger than 1. Each R1 can be any suitable C₁₋₁₀ alkyl group, branched or (for C₃₋₁₀) unbranched, including, but not limited to, methyl, ethyl, propyl, and butyl.

The present invention also provides a compound obtained by use of a method according to the invention.

The present invention also provides a compound obtainable by use of a method according to the invention.

Compounds of the present invention exert growth hormone activity and may as such be used in the treatment of diseases or states which will benefit from an increase in the amount of circulating growth hormone. In particular, the invention provides a method for the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; traumatic spinal cord injury; subarachnoid haemorrhage; very low birth weight; metabolic syndrome; glucocorticoid myopathy; or short stature due to glucucorticoid treatment inchildren, the method comprising administering to a patient in need thereof a therapeutically effective amount of a compound according to the present invention. In particular, said compound is a compound of formula [Va] or formula [VIa].

The terms “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The patient to be treated is preferably a mammal, in particular a human being, but it may also include animals, such as dogs, cats, cows, sheep and pigs. Nonetheless, it should be recognized that therapeutic regimens and prophylactic (preventative) regimens represent separate aspects of the invention.

A “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on e.g. the severity of the disease or injury as well as the weight, sex, age and general state of the subject. It will be understood that determining an appropriate dosage may be achieved using routine experimentation, by constructing a matrix of values and testing different points in the matrix, which is all within the ordinary skills of a trained physician or veterinary.

The present invention thus provides a compound according to the invention for use in therapy.

In one aspect, the invention provides a method for the acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound of the present invention. In particular, said compound is a compound of formula [Va] or formula [VIa].

In one embodiment, the invention relates to the use of compounds according to the present invention in the manufacture of diseases benefiting from an increase in the growth hormone plasma level, such as the disease mentioned above. In particular, said compound is a compound of formula [Va] or formula [VIa].

A typical parenteral dose is in the range of 10⁻⁹ mg/kg to about 100 mg/kg body weight per administration. Typical administration doses are from about 0.0000001 to about 10 mg/kg body weight per administration. The exact dose will depend on e.g. indication, medicament, frequency and mode of administration, the sex, age and general condition of the subject to be treated, the nature and the severity of the disease or condition to be treated, the desired effect of the treatment and other factors evident to the person skilled in the art.

Typical dosing frequencies are twice daily, once daily, bi-daily, twice weekly, once weekly or with even longer dosing intervals. Due to the prolonged half-lifes of the compounds of the present invention compared to the corresponding un-conjugated growth hormone, a dosing regime with long dosing intervals, such as twice weekly, once weekly or with even longer dosing intervals is a particular embodiment of the invention.

Many diseases are treated using more than one medicament in the treatment, either concomitantly administered or sequentially administered. It is therefore within the scope of the present invention to use compounds of the present invention in therapeutic methods for the treatment of one of the above mentioned diseases in combination with one or more other therapeutically active compound normally used in the treatment said diseases. By analogy, it is also within the scope of the present invention to use compounds of the presently invention in combination with other therapeutically active compounds normally used in the treatment of one of the above mentioned diseases in the manufacture of a medicament for said disease.

### Pharmaceutical Compositions

Another purpose is to provide a pharmaceutical composition comprising a compound of the present invention which is present in a concentration from 10⁻¹⁵ mg/ml to 200 mg/ml, such as e.g. 10⁻¹⁰ mg/ml to 5 mg/ml and wherein said composition has a pH from 2.0 to 10.0. The composition may further comprise a buffer system, preservative(s), tonicity agent(s), chelating agent(s), stabilizers and surfactants. In one embodiment of the invention the pharmaceutical composition is an aqueous composition, i.e. composition comprising water. Such composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical composition is a freeze-dried composition, whereto the physician or the patient adds solvents and/or diluents prior to use.

In another embodiment the pharmaceutical composition is a dried composition (e.g. freeze-dried or spray-dried) ready for use without any prior dissolution.

In a further aspect the invention relates to a pharmaceutical composition comprising an aqueous solution of a compound of the present invention, and a buffer, wherein said compound is present in a concentration from 0.1-100 mg/ml or above, and wherein said composition has a pH from about 2.0 to about 10.0.

In a another embodiment of the invention the pH of the composition is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the composition further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 20^{th} edition, 2000.

In a further embodiment of the invention the composition further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects obtained using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 20^{th} edition, 2000.

In a further embodiment of the invention the composition further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 20^{th} edition, 2000.

In a further embodiment of the invention the composition further comprises a stabilizer. The use of a stabilizer in pharmaceutical compositions is well-known to the skilled person. For convenience reference is made to Remington: The Science and Practice of Pharmacy, 20^{th} edition, 2000.

More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In a further embodiment of the invention the composition further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a nonionic surfactant, which protects the protein against aggregation associated with freeze-thawing or mechanical shearing.

In a further embodiment of the invention the composition further comprises a surfactant. In a further embodiment of the invention the surfactant is selected from a detergent, ethoxylated castor oil, polyglycolyzed glycerides, acetylated monoglycerides, sorbitan fatty acid esters, polyoxypropylene-polyoxyethylene block polymers (e.g. poloxamers such as Pluronic® F68, poloxamer 188 and 407, Triton X-100), polyoxyethylene sorbitan fatty acid esters, polyoxyethylene and polyethylene derivatives such as alkylated and alkoxylated derivatives (tweens, e.g. Tween-20, Tween-40, Tween-80 and Brij-35), monoglycerides or ethoxylated derivatives thereof, diglycerides or polyoxyethylene derivatives thereof, alcohols, glycerol, lectins and phospholipids (e.g. phosphatidyl serine, phosphatidyl choline, phosphatidyl ethanolamine, phosphatidyl inositol, diphosphatidyl glycerol and sphingomyelin), derivates of phospholipids (e.g. dipalmitoyl phosphatidic acid) and lysophospholipids (e.g. palmitoyl lysophosphatidyl-L-serine and 1-acyl-sn-glycero-3-phosphate esters of ethanolamine, choline, serine or threonine) and alkyl, alkoxyl(alkyl ester), alkoxy(alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (e.g. cephalins), glyceroglycolipids (e.g. galactopyransoide), sphingoglycolipids (e.g. ceramides, gangliosides), dodecylphosphocholine, hen egg lysolecithin, fusidic acid derivatives—(e.g. sodium tauro-dihydrofusidate etc.), long-chain fatty acids and salts thereof C₆-C₁₂ (e.g. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulphate or sodium lauryl sulphate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates, 3-cholamido-1-propyldimethylammonio-1-propanesulfonate, cationic surfactants (quaternary ammonium bases) (e.g. cetyl-trimethylammonium bromide, cetylpyridinium chloride), non-ionic surfactants (e.g. Dodecyl β-D-glucopyranoside), poloxamines (e.g. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 20^{th} edition, 2000.

It is possible that other ingredients may be present in the pharmaceutical composition of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical composition of the present invention.

Pharmaceutical compositions containing a compound of the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the GH conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the composition of solids, semisolids, powder and solutions for pulmonary administration of a compound of formula [Va] or formula [VIa] using, for example a metered dose inhaler, dry powder inhaler and a nebulizer, all being devices well known to those skilled in the art.

Compositions of the current invention are specifically useful in the composition of controlled, sustained, protracting, retarded, and slow release drug delivery systems. More specifically, but not limited to, compositions are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, cocrystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound of the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized composition” refers to a composition with increased physical stability, increased chemical stability or increased physical and chemical stability.

The term “physical stability” of the protein composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein compositions is evaluated by means of visual inspection and/or turbidity measurements after exposing the composition filled in suitable containers (e.g. cartridges or vials) to mechanical/physical stress (e.g. agitation) at different temperatures for various time periods. Visual inspection of the compositions is performed in a sharp focused light with a dark background. The turbidity of the composition is characterized by a visual score ranking the degree of turbidity for instance on a scale from 0 to 3 (a composition showing no turbidity corresponds to a visual score 0, and a composition showing visual turbidity in daylight corresponds to visual score 3). A composition is classified physical unstable with respect to protein aggregation, when it shows visual turbidity in daylight. Alternatively, the turbidity of the composition can be evaluated by simple turbidity measurements well-known to the skilled person. Physical stability of the aqueous protein compositions can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein composition as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein composition can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized composition” refers to a composition with increased physical stability, increased chemical stability or increased physical and chemical stability. In general, a composition must be stable during use and storage (in compliance with recommended use and storage conditions) until the expiration date is reached.

In one embodiment of the invention the pharmaceutical composition comprising a compound of the present invention is stable for more than 6 weeks of usage and for more than 3 years of storage.

In another embodiment of the invention the pharmaceutical composition comprising the compound of the present invention is stable for more than 4 weeks of usage and for more than 3 years of storage.

In a further embodiment of the invention the pharmaceutical composition comprising the compound of the present invention is stable for more than 4 weeks of usage and for more than two years of storage.

In an even further embodiment of the invention the pharmaceutical composition comprising the compound of the present invention is stable for more than 2 weeks of usage and for more than two years of storage.

The following is a numbered list of embodiments and should not be construed as limiting the invention:

Embodiment 1. A method for covalently attaching PEG to a polypeptide comprising at least one glutamine residue, said method comprising reacting in one or more steps such glutamine residue comprising polypeptide represented by formula [I]

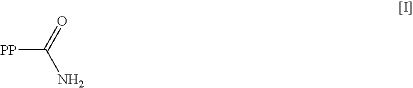

wherein PP represents a polypeptide radical obtained by removing —C(═O)—NH₂ from the side chain of a glutamine residue present in the polypeptide, with a nitrogen containing nucleophile of formula [II]

H₂N-D-R—X [II]

wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated polypeptide of formula [III]

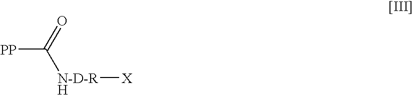

optionally, if X is a latent group, transforming said latent group into —O—NH₂, an aldehyde or a ketone,
said transaminated polypeptide being further reacted with a second compound of formula [IV]

Y—Z [IV]

wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst

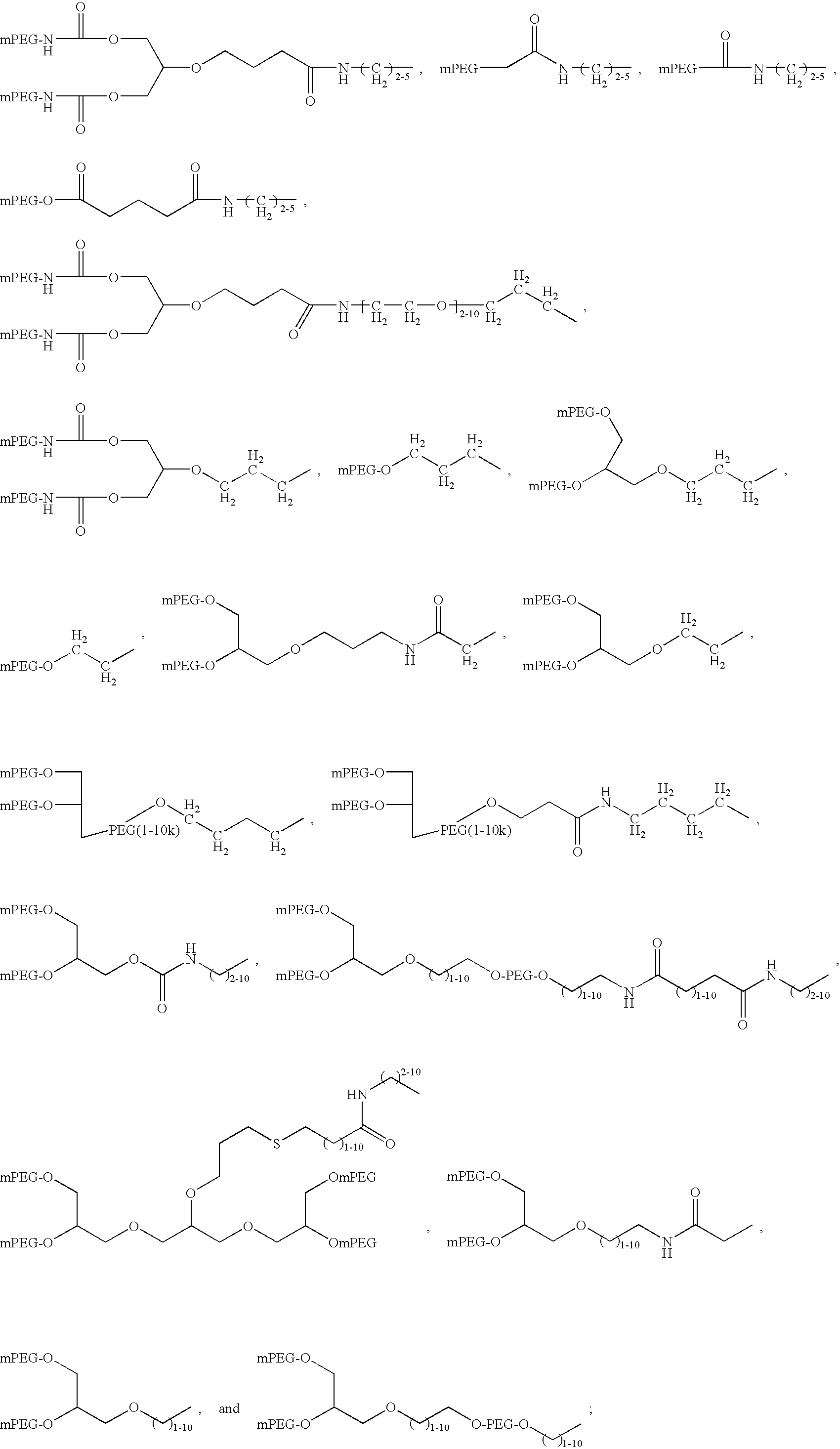

wherein, unless otherwise indicated, mPEG indicates a mPEG with a molecular weight of between 5 kDa and 40 kDa and PEG indicates a PEG with a molecular weight between 1 kDa and 10 kDa; provided that if Z is

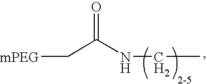

then PEG is 10 kDa PEG to form a PEGylated polypeptide of formula [V]

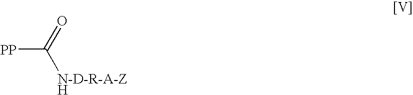

wherein A represents an oxime bond;
or any pharmaceutically acceptable salt, prodrug or solvate thereof.

Embodiment 2. The method according to embodiment 1, wherein D represents —O—.

Embodiment 3. The method according to embodiment 1, wherein D represents a single bond.

Embodiment 4. The method according to any of embodiments 1 to 3, wherein R represents —(CH₂)₄—CH(NH₂)—CO—NH—CH₂— or —(CH₂)₄—CH(NHCOCH₃)—CO—NH—CH₂—.

Embodiment 5. The method according to any of embodiments 1 to 3, wherein R represents C₁₋₆alkylene.

Embodiment 6. The method according to embodiment 5, wherein R represents C₁₋₃alkylene.

Embodiment 7. The method according to embodiment 6, wherein R represents methylene or propylene.

Embodiment 8. The method according to any of embodiments 1 to 7, wherein Z represents

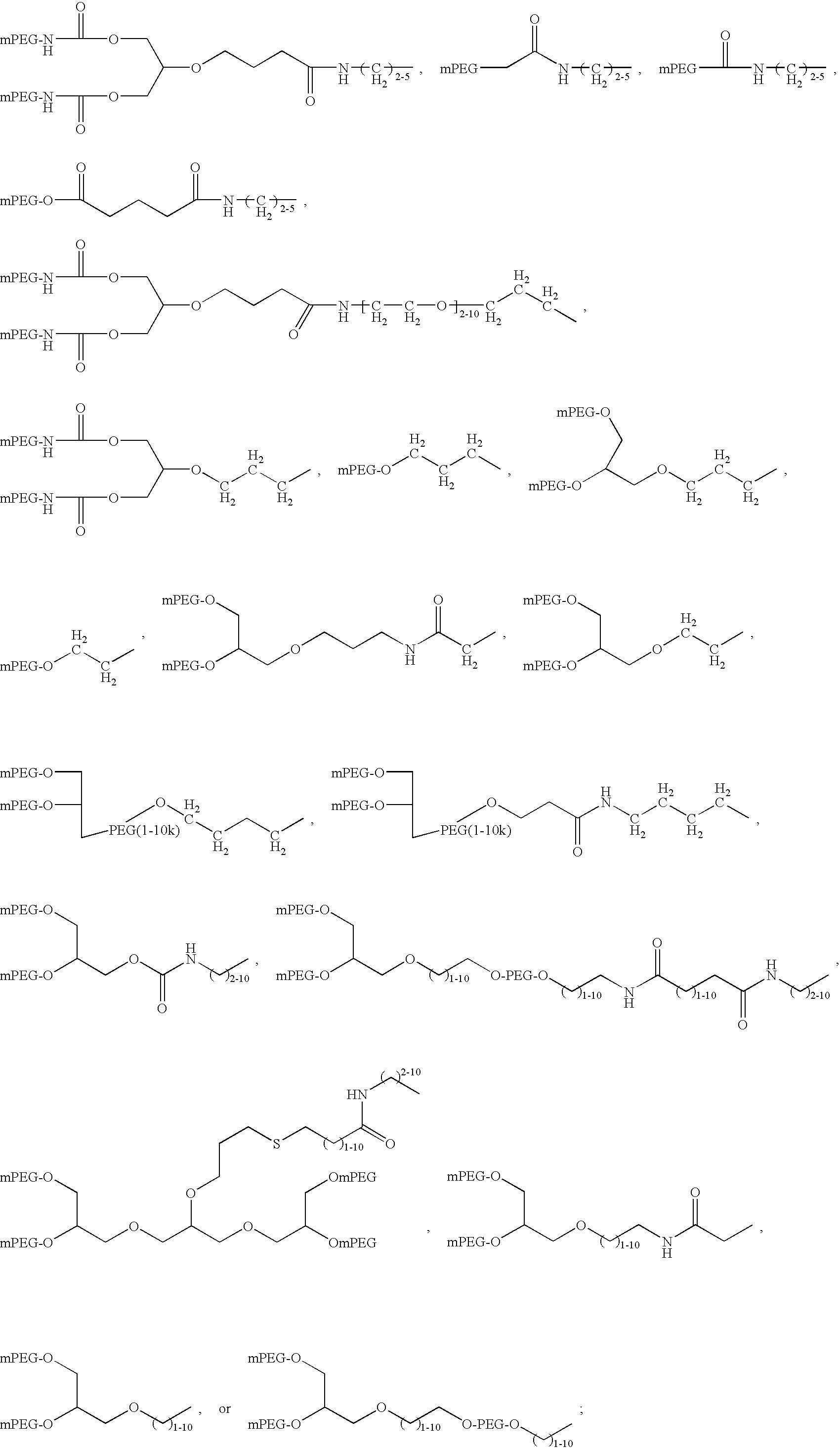

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

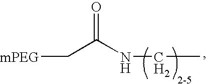

then PEG is 10 kDa PEG.

Embodiment 9. The method according to any of embodiments 1 to 8, wherein Y represents —O—NH₂ and X represents an aldehyde or a latent group, which may be further reacted to form an aldehyde.

Embodiment 10. The method according to any of embodiments 1 to 8, wherein Y represents —O—NH₂ and X represents a ketone or a latent group which may be further reacted to form a ketone.

Embodiment 11. The method according to embodiment 9 or embodiment 10, wherein the compound of formula [IV]

Y—Z [IV]

represents a compound selected from

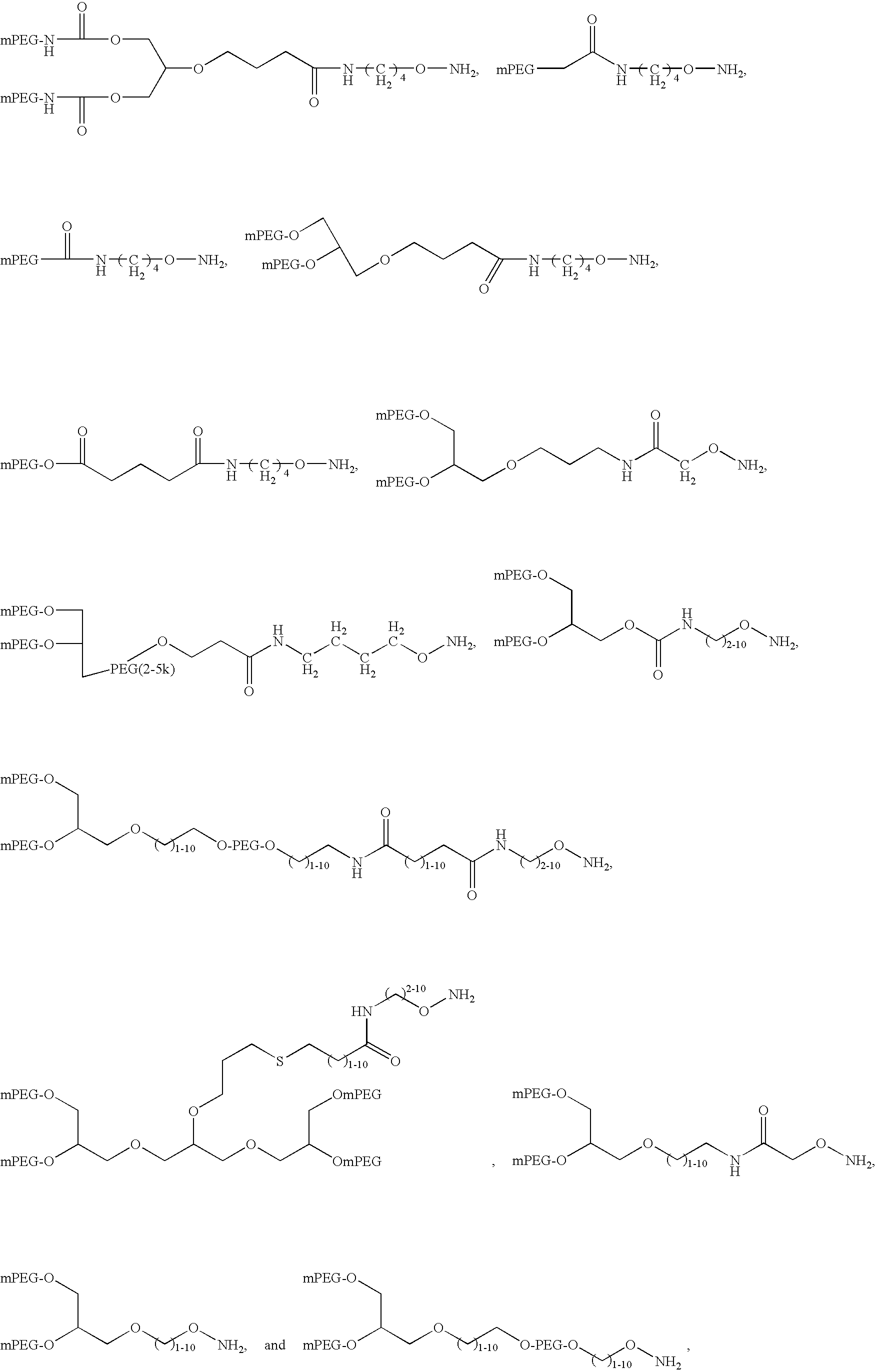

wherein, unless otherwise indicated, mPEG means mPEG with a molecular weight of 10 kDa, 20 kDa or 30 kDa and PEG means PEG with a molecular weight between 2 kDa and 5 kDa.

Embodiment 12. The method according to any of embodiments 1 to 8, wherein Y represents an aldehyde and X represent —O—NH₂ or a latent group which upon further reaction may be transformed into —O—NH₂.

Embodiment 13. The method according to embodiment 12, wherein the compound of formula [IV]

Y—Z [IV]

represents a compound selected from

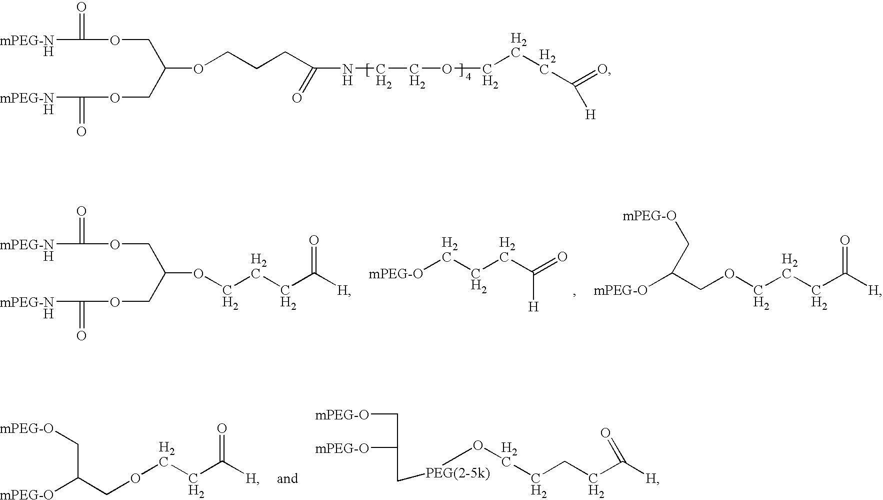

wherein, unless otherwise indicated, mPEG means mPEG with a molecular weight of 10 kDa, 20 kDa or 30 kDa and PEG means PEG with a molecular weight between 2 kDa and 5 kDa.

Embodiment 14. The method according to any of embodiments 1 to 8, wherein Y represents a ketone and X represent —O—NH₂ or a latent group which upon further reaction may be transformed into —O—NH₂.

Embodiment 15. The method according to embodiment 14, wherein the compound of formula [IV]

Y—Z [IV]

represents a compound selected from

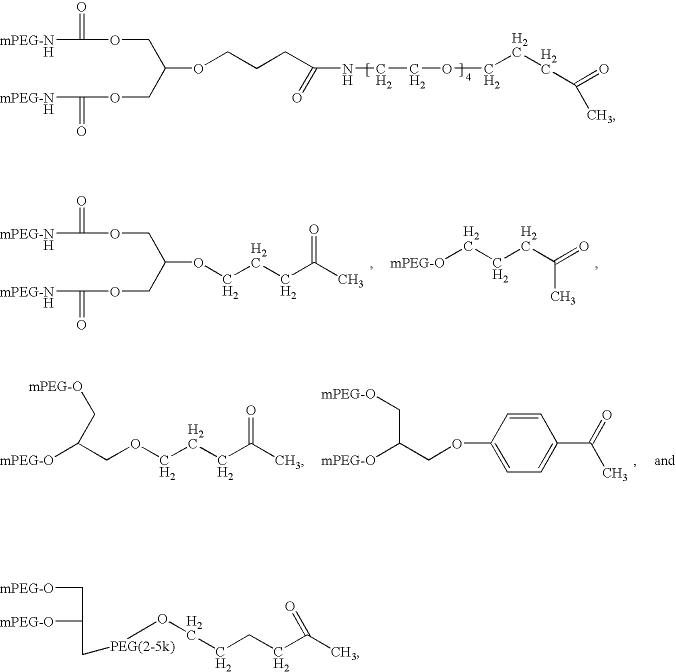

wherein, unless otherwise indicated, mPEG means mPEG with a molecular weight of 10 kDa, 20 kDa or 30 kDa and PEG means PEG with a molecular weight between 2 kDa and 5 kDa.

Embodiment 16. The method according to any of embodiments 1 to 8, wherein the compound of formula [II]

H₂N-D-R—X [II]

represents 1,3-diamino-2-propanol, and

Y represents —O—NH₂.

Embodiment 17. The method according to any of embodiments 1 to 8, wherein the compound of formula [II]

H₂N-D-R—X [II]

represents 1,3-diaminooxy propane, and
Y represents an aldehyde.

Embodiment 18. A method of modifying pharmacological properties of growth hormone, the method comprising covalently attaching PEG to said growth hormone according to a method of any one of embodiments 1 to 17.

Embodiment 19. The method according to according to any of embodiments 1 to 18, wherein the polypeptide is a glutamine residue comprising growth hormone and PP represents a growth hormone radical obtained by removing —C(═O)—NH₂ from the side chain of a glutamine residue present in growth hormone.

Embodiment 20. The method according to embodiment 19, wherein said glutamine residue comprising growth hormone represents a human growth hormone.

Embodiment 21. The method according to embodiment 20, wherein said glutamine residue comprising growth hormone represents
a) a hGH comprising the amino acid sequence of SEQ ID No. 1,
b) 20 kDa hGH,
c) a hGH in which the glutamine residue in the position corresponding to position 40 in SEQ ID No. 1 has been deleted or substituted with another amino acid,
d) a hGH in which the glutamine residue in the position corresponding to position 141 in SEQ ID No. 1 has been deleted or substituted with another amino acid, or
e) a hGH in which the glutamine residue in the position corresponding to position 40 in SEQ ID No. 1, and the glutamine residue in the position corresponding to position 141 in SEQ ID No. 1 each have been deleted or substituted with another amino acid, and where a glutamine residue is present in another position in the growth hormone.

Embodiment 22. The method according to embodiment 21, wherein said growth hormone represents hGH.

Embodiment 23. A compound according to formula [V]

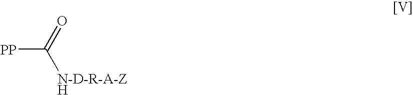

wherein PP represents a polypeptide radical obtained by removing —C(═O)—NH₂ from the side chain of a glutamine residue present in the polypeptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂— or C₅₋₁₅heteroalkylene;
A represents an oxime bond;
Z represents a moiety selected amongst

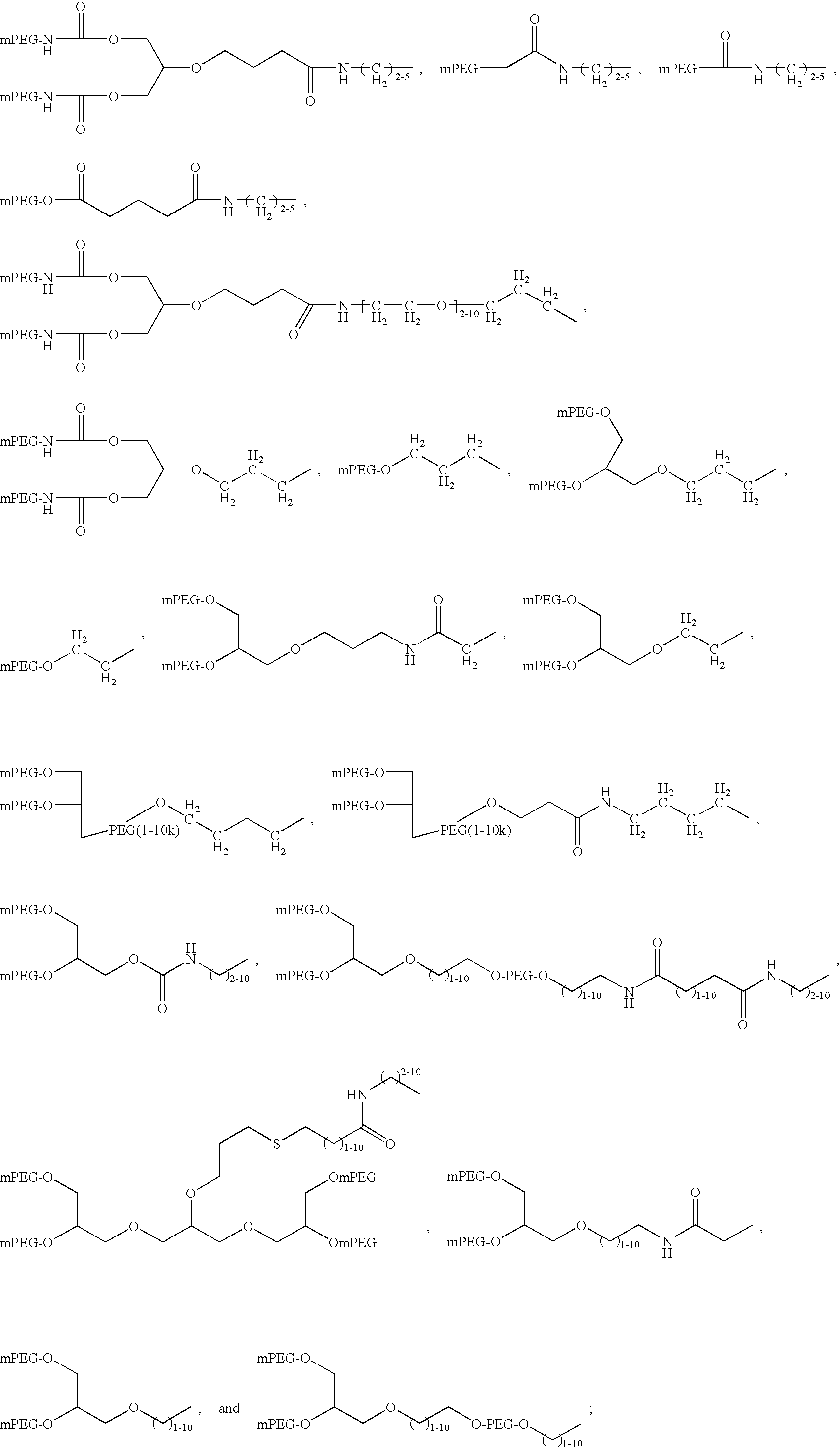

wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof;
provided that if Z is

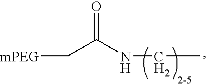

then mPEG is 10 kDa mPEG.

Embodiment 24. The compound according to embodiment 23, wherein D represents —O—.

Embodiment 25. The compound according to embodiment 23, wherein D represents a single bond.

Embodiment 26. The compound according to any of embodiments 23 to 25, wherein R represents —(CH₂)₄—CH(NH₂)—CO—NH—CH₂— or —(CH₂)₄—CH(NHCOCH₃)—CO—NH—CH₂—.

Embodiment 27. The compound according to any of embodiments 23 to 25, wherein R represents C₁₋₆alkylene.

Embodiment 28. The compound according to embodiment 27, wherein R represents C₁₋₃alkylene.

Embodiment 29. The compound according to embodiment 28, wherein R represents methylene or propylene.

Embodiment 30. The compound according to any of embodiments 23 to 29, wherein Z represents

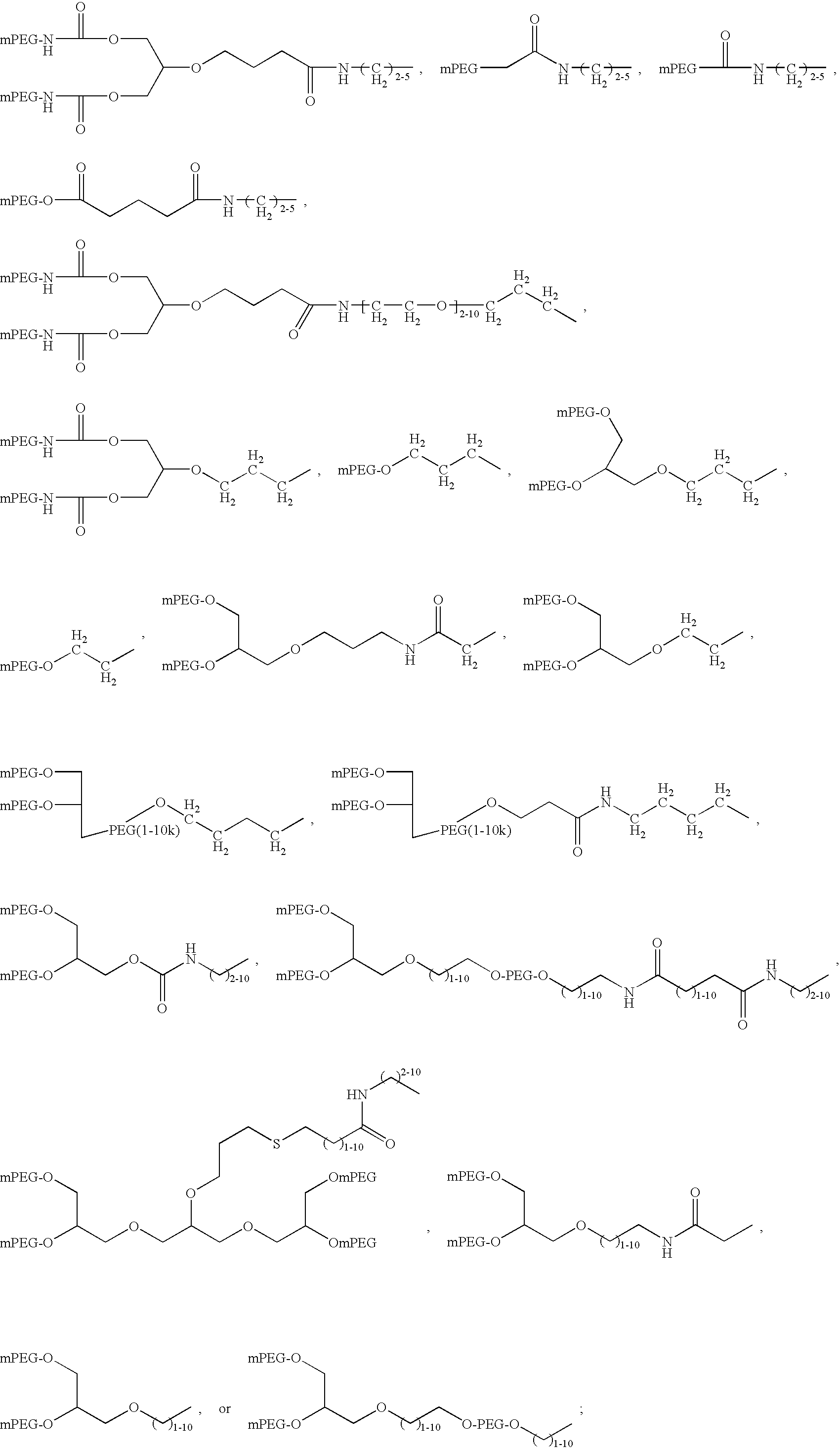

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

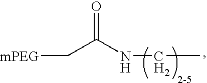

then PEG is 10 kDa PEG.

Embodiment 31. The compound according to any of embodiments 23 to 30, wherein the polypeptide is a glutamine residue comprising growth hormone and PP represents a growth hormone radical obtained by removing —C(═O)—NH₂ from the side chain of a glutamine residue present in growth hormone.

Embodiment 32. The compound according to embodiment 31, wherein GH represents the radical obtained by removing —C(═O)—NH₂ from the side chain of a glutamine residue present in an hGH comprising the amino acid sequence of SEQ ID No. 1.

Embodiment 33. The compound according to embodiment 31, wherein GH represents the radical obtained by removing —C(═O)—NH₂ from the side chain of a glutamine residue present in 20 kDa hGH.

Embodiment 34. The compound according to embodiment 31, wherein GH represents the radical obtained by removing —C(═O)—NH₂ from the side chain of
a) the glutamine residue in the position corresponding to position 40 in SEQ ID No. 1; or
b) the glutamine residue in the position corresponding to position 141 in SEQ ID No. 1; or
   GH represents the radical obtained by removing —C(═O)—NH₂ from the side chain of a glutamine residue in the position corresponding to position 40 in SEQ ID No. 1, wherein the glutamine residue in the position corresponding to position 141 in SEQ ID No. 1 has been deleted or substituted with another amino acid; or
   GH represents the radical obtained by removing —C(═O)—NH₂ from the side chain of a glutamine residue in the position corresponding to position 141 in SEQ ID No. 1, wherein the glutamine residue in the position corresponding to position 40 in SEQ ID No. 1 has been deleted or substituted with another amino acid; or
   GH represents the radical obtained by removing —C(═O)—NH₂ from the side chain of a glutamine residue present in a position different from the position corresponding to position 40 in SEQ ID No. 1 and different from the position corresponding to position 141 in SEQ ID No. 1, wherein the glutamine residue in the position corresponding to position 40 in SEQ ID No. 1, and the glutamine residue in the position corresponding to position 141 in SEQ ID No. 1 each have been deleted or substituted with another amino acid.

Embodiment 35. A compound according to embodiment 23 selected from
N^{δ141/40}-2-(O-(4-{4-(1,3-bis(mPEG(10k)ylaminocarbonyloxy)prop-2-yloxy)butyryl}aminobutyl)oximino)ethyl hGH;
N^{δ141/40}-2-(O-(4-{4-(mPEG(10k)yloxy)butyryl}aminobutyl)oximino)ethyl hGH;
N^{δ141/40}-2-(O-(4-{3-(mPEG(10k)yloxy)propionyl}aminobutyl)oximino)ethyl hGH;
N^{δ141/40}-2-(O-(4-{4-(2,3-bis(mPEG(10k)yloxy)prop-1-yloxy)butyryl}aminobutyl)oximino)ethyl hGH;
N^{δ141/40}-2-(O-(4-{5-(mPEG(10k)yloxy-5-oxopentanoyl}aminobutyl)oximino)ethyl hGH;
N^{δ141/40}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(10k)ylaminocarbonyloxy)prop-2-yloxy)butylidene}-aminoxy)propyloxy hGH;
N^{δ141/40}-3-({4-(mPEG(10k)yloxy)butylidene}aminoxy)propyloxy hGH;
N^{δ141/40}-3-({4-(2,3-bis(mPEG(10k)yloxy)prop-1-yloxy)butylidene}aminoxy)propyloxy hGH;
N^{δ141/40}-3-({3-(mPEG(10k)yloxy)propylidene}aminoxy)propyloxy hGH;
N^{δ141/40}-2-(O-(2-(3-(2,3-bis(mPEG(10k)yloxy)propyloxy)propylamino)-2-oxoethyl)oximino)ethyl hGH;
N^{δ141/40}-3-({4-(2,3-bis(mPEG(10k)yloxy)prop-1-yloxy)propylidene}aminoxy)propyloxy hGH;
N^{δ141/40}-2-(O-(4-{4-(1,3-bis(mPEG(20k)ylaminocarbonyloxy)prop-2-yloxy)butyryl}aminobutyl)oximino)ethyl hGH;
N^{δ141/40}-2-(O-(4-{3-(mPEG(20k)yloxy)propionyl}aminobutyl)oximino)ethyl hGH;
N^{δ141/40}-2-(O-(4-{4-(2,3-bis(mPEG(20k)yloxy)prop-1-yloxy)butyryl}aminobutyl)oximino)ethyl hGH;
N^{δ141/40}-2-(O-(4-{5-(mPEG(20k)yloxy-5-oxopentanoyl}aminobutyl)oximino)ethyl hGH;
N^{δ141/40}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(20k)ylaminocarbonyloxy)prop-2-yloxy)butylidene}-aminoxy)propyloxy hGH;
N^{δ141/40}-3-({4-(mPEG(20k)yloxy)butylidene}aminoxy)propyloxy hGH;
N^{δ141/40}-3-({4-(2,3-bis(mPEG(20k)yloxy)prop-1-yloxy)butylidene}aminoxy)propyloxy hGH;
N^{δ141/40}-3-({3-(mPEG(20k)yloxy)propylidene}aminoxy)propyloxy hGH;
N^{δ141/40}-2-(O-(2-(3-(2,3-bis(mPEG(20k)yloxy)propyloxy)propylamino)-2-oxoethyl)oximino)ethyl hGH;
N^{δ141/40}-3-({4-(2,3-bis(mPEG(20k)yloxy)prop-1-yloxy)propylidene}aminoxy)propyloxy hGH;
N^{δ141/40}-2-(O-(4-{4-(1,3-bis(m PEG(30k)ylaminocarbonyloxy)prop-2-yloxy)butyryl}aminobutyl)oximino)ethyl hGH;
N^{δ141/40}-2-(O-(4-{3-(mPEG(30k)yloxy)propionyl}aminobutyl)oximino)ethyl hGH;
N^{δ141/40}-2-(O-(4-{4-(2,3-bis(mPEG(30k)yloxy)prop-1-yloxy)butyryl}aminobutyl)oximino)ethyl hGH;
N^{δ141/40}-2-(O-(4-{5-(m PEG(30k)yloxy-5-oxopentanoyl}aminobutyl)oximino)ethyl hGH;
N^{δ141/40}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(m PEG(30k)ylaminocarbonyloxy)prop-2-yloxy)butylidene}-aminoxy)propyloxy hGH;
N^{δ141/40}-3-({4-(m PEG(30k)yloxy)butylidene}aminoxy)propyloxy hGH;
N^{δ141/40}-3-({4-(2,3-bis(mPEG(30 k)yloxy)prop-1-yloxy)butylidene}aminoxy)propyloxy hGH;
N^{δ141/40}-3-({3-(mPEG(30k)yloxy)propylidene}aminoxy)propyloxy hGH;
N^{δ141/40}-2-(O-(2-(3-(2,3-bis(mPEG(30k)yloxy)propyloxy)propylamino)-2-oxoethyl)oximino)ethyl hGH;
N^{δ141/40}-3-({4-(2,3-bis(mPEG(30k)yloxy)prop-1-yloxy)propylidene}aminoxy)propyloxy hGH;
N^{δ141/40}-3-({4-{(2,3-bis(mPEG(20k)yl)prop-1-yloxy)PEGyloxy}butylidene}aminoxy)propyloxy hGH;
N^{δ141/40}-2-((4-(4-((2,3-bis(mPEG(20k)yl)propyl)PEGyloxy)butyrylamino)butyl)oximino)ethyl hGH;
N^{δ141}-2-(O-(4-{4-(1,3-bis(mPEG(10k)ylaminocarbonyloxy)prop-2-yloxy)butyryl}-aminobutyl)oximino)ethyl hGH;
N^{δ141}-2-(O-(4-{4-(mPEG(10k)yloxy)butyryl}aminobutyl)oximino)ethyl hGH;
N^{δ141}-2-(O-(4-{3-(mPEG(10k)yloxy)propionyl}aminobutyl)oximino)ethyl hGH;
N^{δ141}-2-(O-(4-{4-(2,3-bis(mPEG(10k)yloxy)prop-1-yloxy)butyryl}aminobutyl)oximino)ethyl hGH;
N^{δ141}-2-(O-(4-{5-(mPEG(10k)yloxy-5-oxopentanoyl}aminobutyl)oximino)ethyl hGH;
N^{δ141}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(10k)ylaminocarbonyloxy)prop-2-yloxy)butylidene}-aminoxy)propyloxy hGH;
N^{δ141}-3-({4-(mPEG(10k)yloxy)butylidene}aminoxy)propyloxy hGH;
N^{δ141}-3-({4-(2,3-bis(mPEG(10k)yloxy)prop-1-yloxy)butylidene}aminoxy)propyloxy hGH;
N^{δ141}-3-({3-(mPEG(10k)yloxy)propylidene}aminoxy)propyloxy hGH;
N^{δ141}-2-(O-(2-(3-(2,3-bis(mPEG(10k)yloxy)propyloxy)propylamino)-2-oxoethyl)oximino)ethyl hGH;
N^{δ141}-3-({4-(2,3-bis(mPEG(10k)yloxy)prop-1-yloxy)propylidene}aminoxy)propyloxy hGH;
N^{δ141}-2-(O-(4-{4-(1,3-bis(mPEG(20k)ylaminocarbonyloxy)prop-2-yloxy)butyryl}-minobutyl)oximino)ethyl hGH;
N^{δ141}-2-(O-(4-{3-(mPEG(20k)yloxy)propionyl}aminobutyl)oximino)ethyl hGH;
N^{δ141}-2-(O-(4-{4-(2,3-bis(mPEG(20k)yloxy)prop-1-yloxy)butyryl}aminobutyl)oximino)ethyl hGH;
N^{δ141}-2-(O-(4-{5-(mPEG(20k)yloxy-5-oxopentanoyl}aminobutyl)oximino)ethyl hGH;
N^{δ141}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(20k)ylaminocarbonyloxy)prop-2-yloxy)butylidene}-aminoxy)propyloxy hGH;
N^{δ141}-3-({4-(mPEG(20k)yloxy)butylidene}aminoxy)propyloxy hGH;
N^{δ141}-3-({4-(2,3-bis(mPEG(20k)yloxy)prop-1-yloxy)butylidene}aminoxy)propyloxy hGH;
N^{δ141}-3-({3-(mPEG(20k)yloxy)propylidene}aminoxy)propyloxy hGH;
N^{δ141}-2-(O-(2-(3-(2,3-bis(mPEG(20k)yloxy)propyloxy)propylamino)-2-oxoethyl)oximino)ethyl hGH;
N^{δ141}-3-({4-(2,3-bis(mPEG(20k)yloxy)prop-1-yloxy)propylidene}aminoxy)propyloxy hGH;
N^{δ141}-2-(O-(4-{4-(1,3-bis(mPEG(30k)ylaminocarbonyloxy)prop-2-yloxy)butyryl}-aminobutyl)oximino)ethyl hGH;
N^{δ141}-2-(O-(4-{3-(mPEG(30k)yloxy)propionyl}aminobutyl)oximino)ethyl hGH;
N^{δ141}-2-(O-(4-{4-(2,3-bis(mPEG(30k)yloxy)prop-1-yloxy)butyryl}aminobutyl)oximino)ethyl hGH,
N^{δ141}-2-(O-(4-{5-(mPEG(30k)yloxy-5-oxopentanoyl}aminobutyl)oximino)ethyl hGH;
N^{δ141}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(30k)ylaminocarbonyloxy)prop-2-yloxy)butylidene}-aminoxy)propyloxy hGH;
N^{δ141}-3-({4-(mPEG(30k)yloxy)butylidene}aminoxy)propyloxy hGH;
N^{δ141}-3-({4-(2,3-bis(mPEG(30k)yloxy)prop-1-yloxy)butylidene}aminoxy)propyloxy hGH;
N^{δ141}-3-({3-(mPEG(30k)yloxy)propylidene}aminoxy)propyloxy hGH;
N^{δ141}-2-(O-(2-(3-(2,3-bis(mPEG(30k)yloxy)propyloxy)propylamino)-2-oxoethyl)oximino)ethyl hGH;
N^{δ141}-3-({4-(2,3-bis(mPEG(30k)yloxy)prop-1-yloxy)propylidene}aminoxy)propyloxy hGH;
N^{δ141}-3-({4-{(2,3-bis(mPEG(20k)yloxy)prop-1-yl)PEGyloxy}butylidene}aminoxy)propyloxy hGH;
N^{δ141}-2-((4-(4-((2,3-bis(mPEG(20k)yl)propyl)PEGyloxy)butyrylamino)butyl)oximino)ethyl hGH;
N^{δ40}-2-(O-(4-{4-(1,3-bis(mPEG(10k)ylaminocarbonyloxy)prop-2-yloxy)butyryl}-aminobutyl)oximino)ethyl hGH;
N^{δ40}-2-(O-(4-{4-(mPEG(10k)yloxy)butyryl}aminobutyl)oximino)ethyl hGH;
N^{δ40}-2-(O-(4-{3-(mPEG(10k)yloxy)propionyl}aminobutyl)oximino)ethyl hGH;
N^{δ40}-2-(O-(4-{4-(2,3-bis(mPEG(10k)yloxy)prop-1-yloxy)butyryl}aminobutyl)oximino)ethyl hGH;
N^{δ40}-2-(O-(4-{5-(mPEG(10k)yloxy-5-oxopentanoyl}aminobutyl)oximino)ethyl hGH;
N^{δ40}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(10k)ylaminocarbonyloxy)prop-2-yloxy)butylidene}-aminoxy)propyloxy hGH;
N^{δ40}-3-({4-(mPEG(10k)yloxy)butylidene}aminoxy)propyloxy hGH;
N^{δ40}-3-({4-(2,3-bis(mPEG(10k)yloxy)prop-1-yloxy)butylidene}aminoxy)propyloxy hGH;
N^{δ40}-3-({3-(mPEG(10k)yloxy)propylidene}aminoxy)propyloxy hGH;
N^{δ40}-2-(O-(2-(3-(2,3-bis(mPEG(10k)yloxy)propyloxy)propylamino)-2-oxoethyl)oximino)ethyl hGH;
N^{δ40}-3-({4-(2,3-bis(mPEG(10k)yloxy)prop-1-yloxy)propylidene}aminoxy)propyloxy hGH;
N^{δ40}-2-(O-(4-{4-(1,3-bis(mPEG(20k)ylaminocarbonyloxy)prop-2-yloxy)butyryl}-aminobutyl)oximino)ethyl hGH;
N^{δ40}-2-(O-(4-{3-(mPEG(20k)yloxy)propionyl}aminobutyl)oximino)ethyl hGH;
N^{δ40}-2-(O-(4-{4-(2,3-bis(mPEG(20k)yloxy)prop-1-yloxy)butyryl}aminobutyl)oximino)ethyl hGH;
N^{δ40}-2-(O-(4-{5-(mPEG(20k)yloxy-5-oxopentanoyl}aminobutyl)oximino)ethyl hGH;
N^{δ40}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(20k)ylaminocarbonyloxy)prop-2-yloxy)butylidene}-aminoxy)propyloxy hGH;
N^{δ40}-3-({4-(mPEG(20k)yloxy)butylidene}aminoxy)propyloxy hGH;
N^{δ40}-3-({4-(2,3-bis(mPEG(20k)yloxy)prop-1-yloxy)butylidene}aminoxy)propyloxy hGH;
N^{δ40}-3-({3-(mPEG(20k)yloxy)propylidene}aminoxy)propyloxy hGH;
N^{δ40}-2-(O-(2-(3-(2,3-bis(mPEG(20k)yloxy)propyloxy)propylamino)-2-oxoethyl)oximino)ethyl hGH,
N^{δ40}-3-({4-(2,3-bis(mPEG(20k)yloxy)prop-1-yloxy)propylidene}aminoxy)propyloxy hGH;
N^{δ40}-2-(O-(4-{4-(1,3-bis(mPEG(30k)ylaminocarbonyloxy)prop-2-yloxy)butyryl}-aminobutyl)oximino)ethyl hGH;
N^{δ40}-2-(O-(4-{3-(mPEG(30k)yloxy)propionyl}aminobutyl)oximino)ethyl hGH;
N^{δ40}-2-(O-(4-{4-(2,3-bis(mPEG(30k)yloxy)prop-1-yloxy)butyryl}aminobutyl)oximino)ethyl hGH;
N^{δ40}-2-(O-(4-{5-(mPEG(30k)yloxy-5-oxopentanoyl}aminobutyl)oximino)ethyl hGH;
N^{δ40}-3-({4-(2-(2-(2-(2-(4-(1,3-bis(mPEG(30k)ylaminocarbonyloxy)prop-2-yloxy)butyrylamino)-ethoxy)ethoxy)ethoxy)ethoxy)butylidene}aminoxy)prop-1-yloxy hGH;
N^{δ40}-3-({4-(1,3-bis(mPEG(30k)ylaminocarbonyloxy)prop-2-yloxy)butylidene}-aminoxy)propyloxy hGH;
N^{δ40}-3-({4-(mPEG(30k)yloxy)butylidene}aminoxy)propyloxy hGH;
N^{δ40}-3-({4-(2,3-bis(mPEG(30k)yloxy)prop-1-yloxy)butylidene}aminoxy)propyloxy hGH;
N^{δ40}-3-({3-(mPEG(30k)yloxy)propylidene}aminoxy)propyloxy hGH;
N^{δ40}-2-(O-(2-(3-(2,3-bis(mPEG(30k)yloxy)propyloxy)propylamino)-2-oxoethyl)oximino)ethyl hGH,
N^{δ40}-3-({4-(2,3-bis(mPEG(30k)yloxy)prop-1-yloxy)propylidene}aminoxy)propyloxy hGH;
N^{δ40}-3-({4-{(2,3-bis(mPEG(20k)yloxy)prop-1-yl)PEGyloxy}butylidene}aminoxy)propyloxy hGH;
N^{δ40}-2-((4-(4-((2,3-bis(mPEG(20k)yl)propyl)PEGyloxy)butyrylamino)butyl)oximino)ethyl hGH;
N^{ε}141-[2-O-(4-(4-(1,3-bis(mPEG(20K)aminocarbonyloxy)-2-propyloxy)butyrylamino)butyl)oxyimino)ethyl]hGH;
N^{ε}141-[2-(O-(4-(4-(mPEG(30K)oxy)butyrylamino)-butyl)oxyimino)-ethyl]hGH;
N^{ε}141-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}141-[2-(2-(2,3-(mPEG(20K)yloxy)propyloxycarbonylamino)ethyloximino)ethyl]hGH;
N^{ε}141-[2-(O-(2-(2-(mPEG(40K)yloxy)ethylamino)-2-oxoethyl)oximino)ethyl]hGH;
N^{ε}141-[2-(3-(4-((1,3-bis(mPEG(30K)ylaminocarbonyloxy)-2-propyloxy)butylidene)aminoxy)propyloxyimino)ethyl]hGH;
N^{ε}141/40-[2-O-(4-(4-(1,3-bis(mPEG(20K)aminocarbonyloxy)-2-propyloxy)butyrylamino)butyl)oxyimino)ethyl]hGH;
N^{ε}141/40-[2-(O-(4-(4-(mPEG(30K)oxy)butyrylamino)-butyl)oxyimino)-ethyl]hGH;
N^{ε}141/40-(3-((4-(2-(2-(2-(2-(4-(1,3-bis(m PEG(20K)ylaminocarbonyloxy)-2-propyloxy)butyrylamino)ethoxy)ethoxy)ethoxy)ethoxy)butylidene)aminoxy)propyloxy)hGH;
N^{ε}141/40-[2-(2-(2,3-(mPEG(20K)yloxy)propyloxycarbonylamino)ethyloximino)ethyl]hGH;
N^{ε}141/40-[2-(O-(2-(2-(mPEG(40K)yloxy)ethylamino)-2-oxoethyl)oximino)ethyl]hGH;
N^{ε}141/40-[2-(3-(4-((1,3-bis(mPEG(30K)ylaminocarbonyloxy)-2-propyloxy)butylidene)aminoxy)propyloxyimino)ethyl]hGH;
N^{ε}40-[2-O-(4-(4-(1,3-bis(mPEG(20K)aminocarbonyloxy)-2-propyloxy)butyrylamino)butyl)oxyimino)ethyl]hGH;
N^{ε}40-[2-(O-(4-(4-(mPEG(30K)oxy)butyrylamino)-butyl)oxyimino)-ethyl]hGH;
N^{ε}40-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}40-[2-(2-(2,3-(mPEG(20K)yloxy)propyloxycarbonylamino)ethyloximino)ethyl]hGH;
N^{ε}40-[2-(O-(2-(2-(mPEG(40K)yloxy)ethylamino)-2-oxoethyl)oximino)ethyl]hGH;
N^{ε}40-[2-(3-(4-((1,3-bis(mPEG(30K)ylaminocarbonyloxy)-2-propyloxy)butylidene)aminoxy)propyloxyimino)ethyl]hGH;

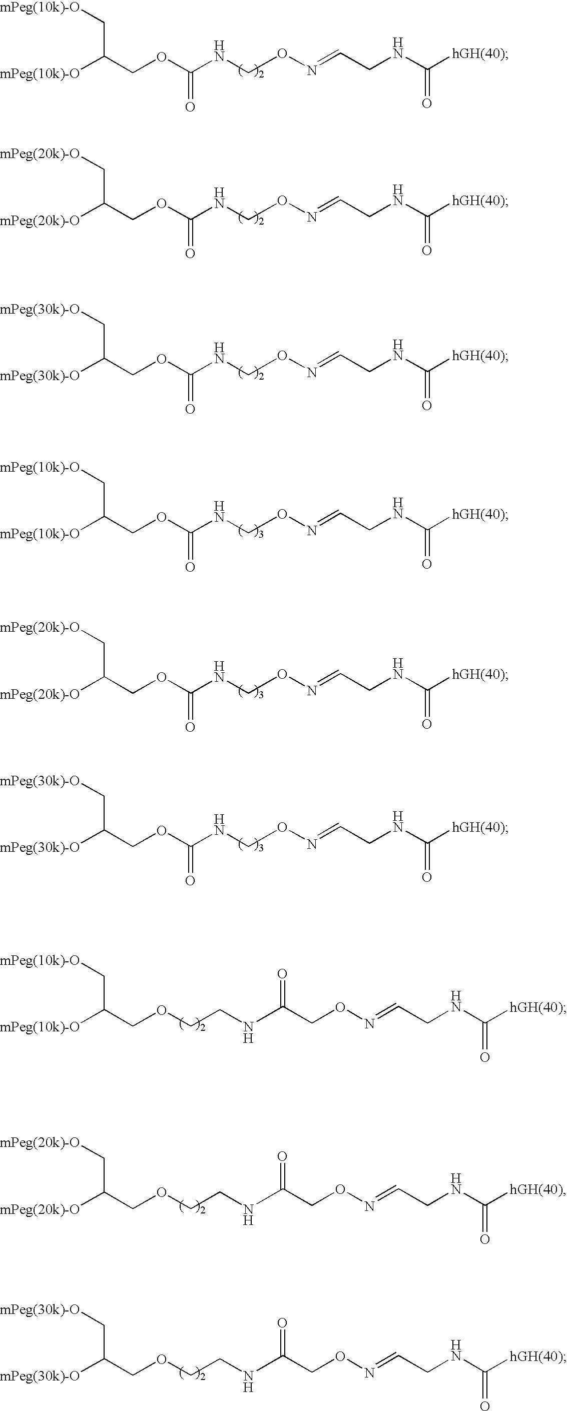

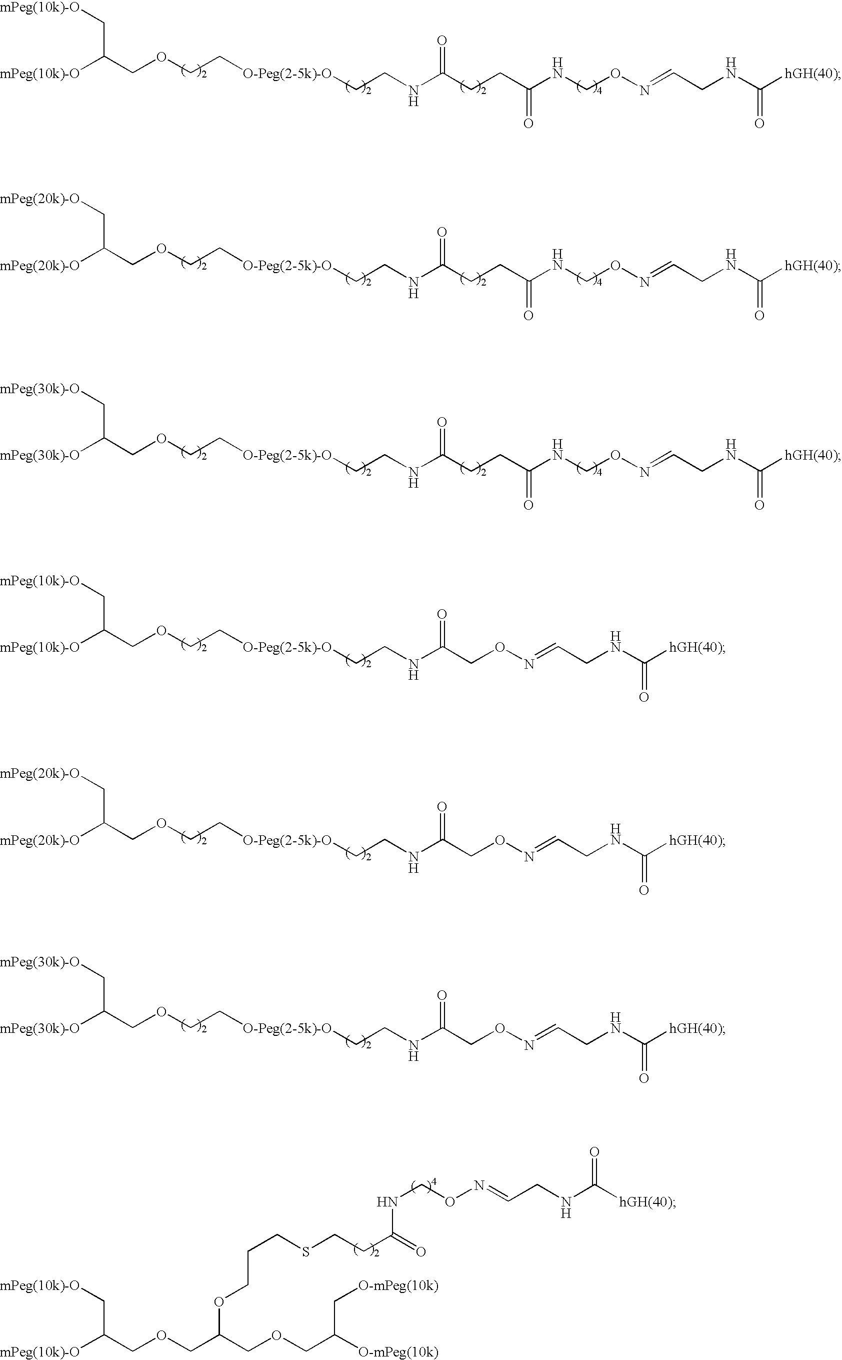

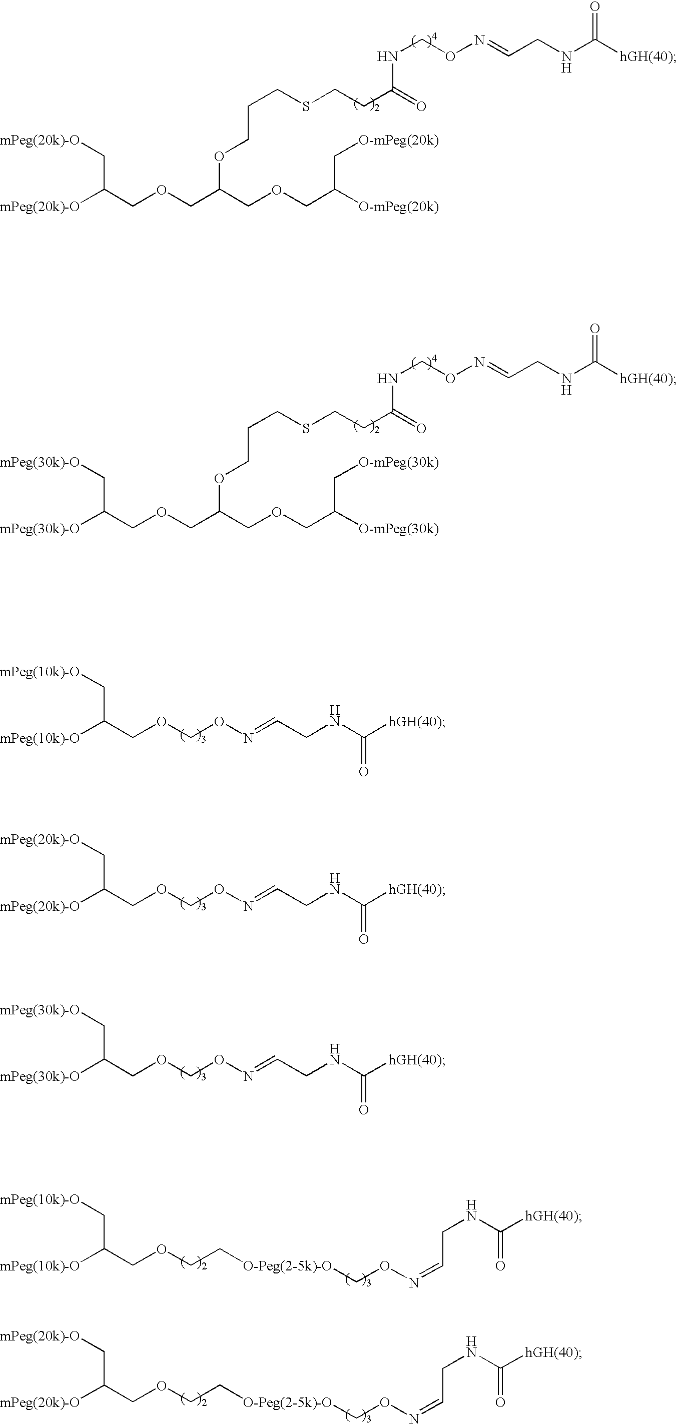

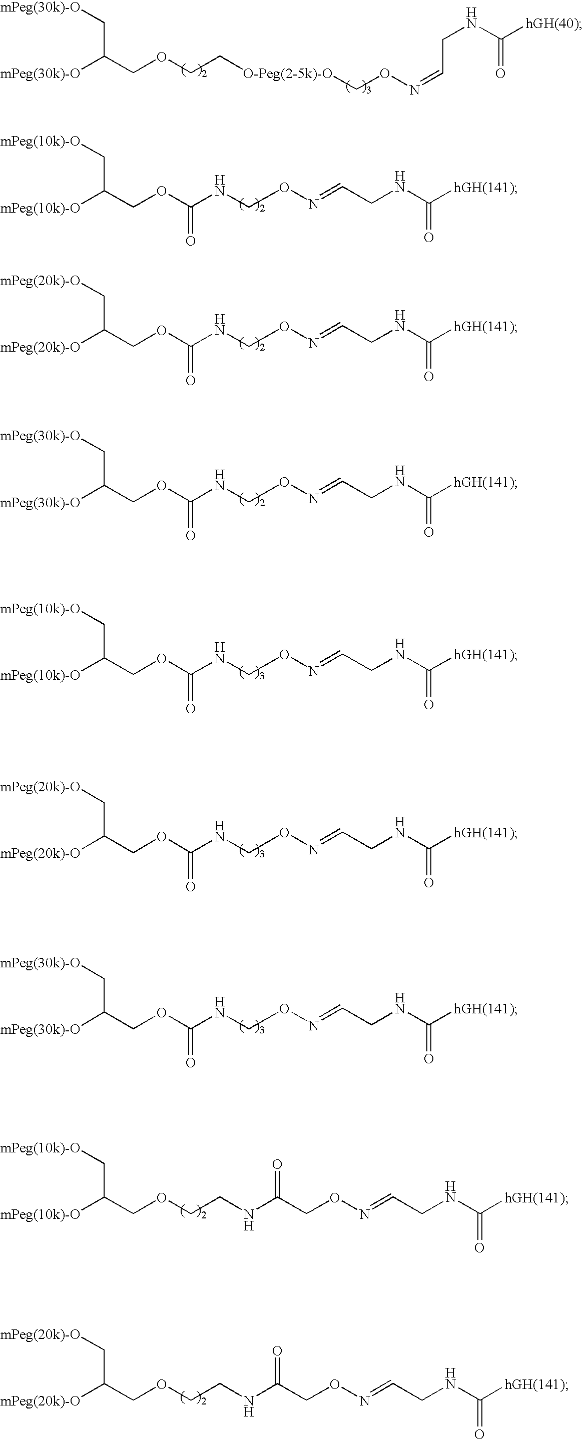

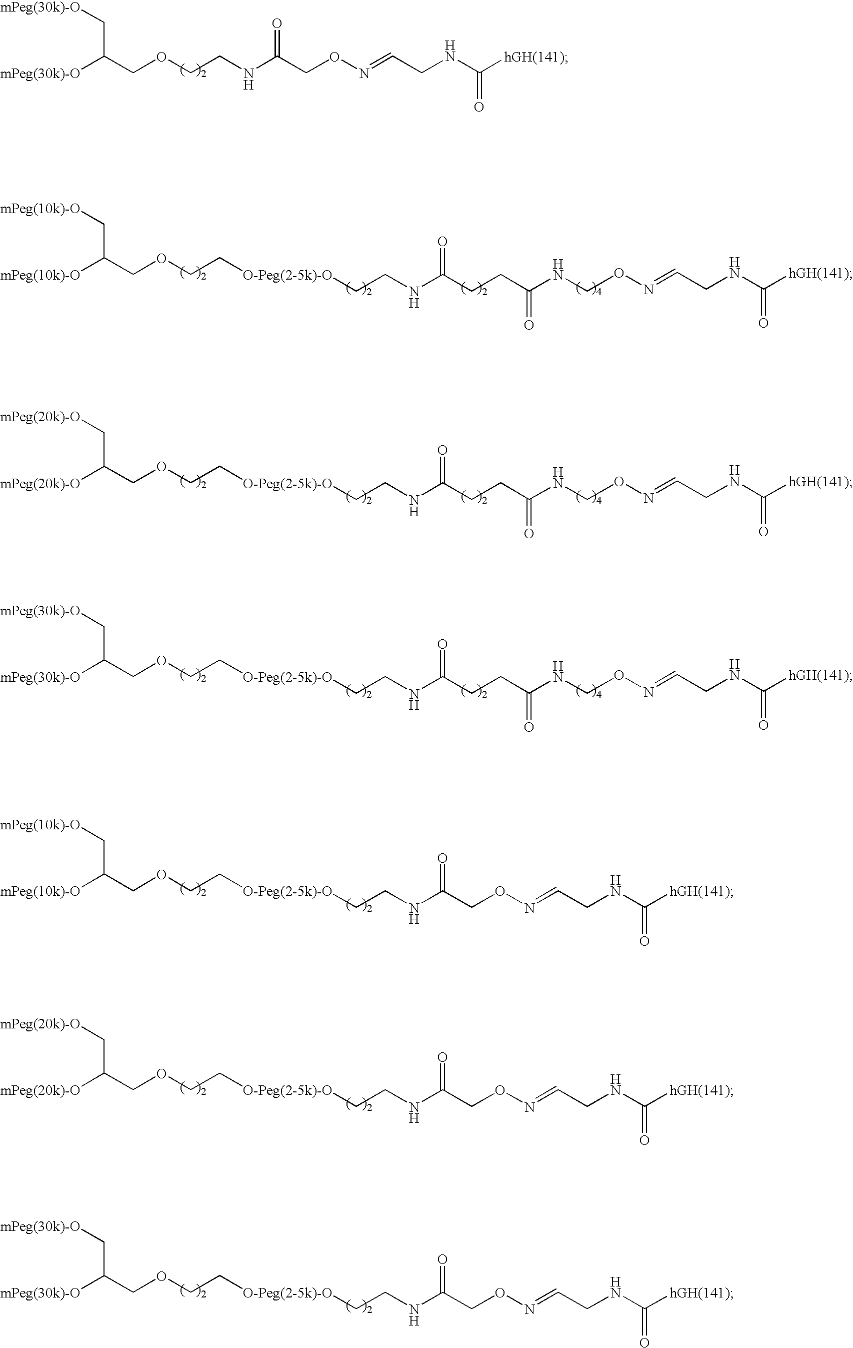

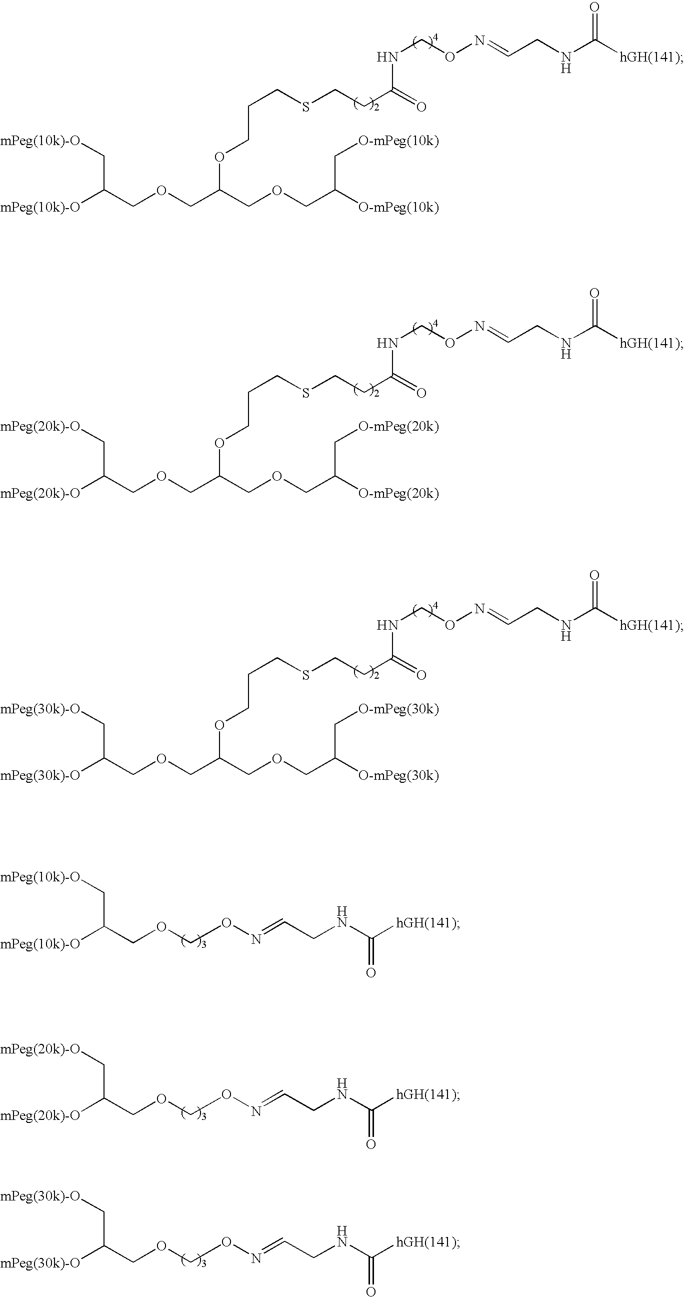

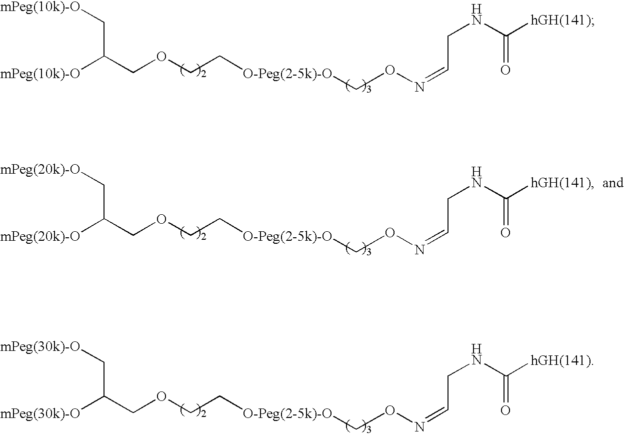

Embodiment 36. A compound according to formula [VI]

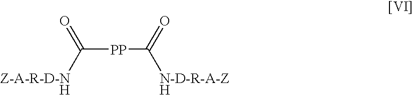

wherein PP represents a polypeptide radical obtained by removing —C(═O)—NH₂ from the side chain of two glutamine residues present in said polypeptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
A represents an oxime bond;
Z represents a moiety selected amongst

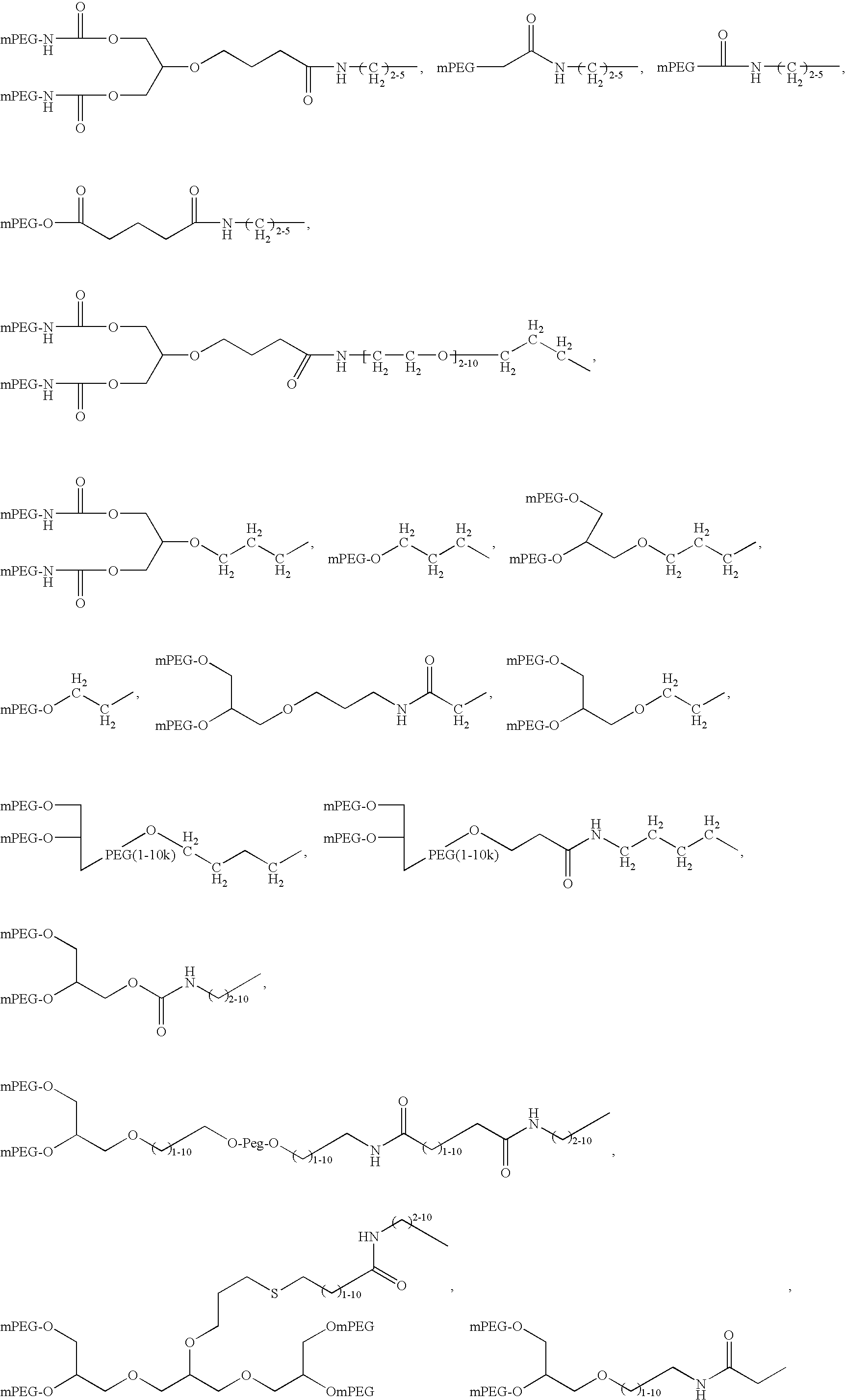

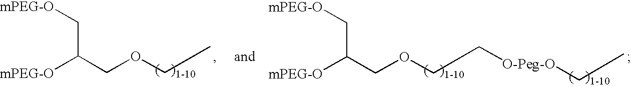

wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof;
provided that if Z is

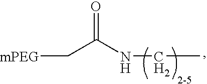

then mPEG is 10 kDa mPEG.

Embodiment 37. The compound according to embodiment 36, wherein D represents —O—.

Embodiment 38. The compound according to embodiment 36, wherein D represents a single bond.

Embodiment 39. The compound according to any of embodiments 36 to 38, wherein R represents —(CH₂)₄—CH(NH₂)—CO—NH—CH₂— or —(CH₂)₄—CH(NHCOCH₃)—CO—NH—CH₂—.

Embodiment 40. The compound according to any of embodiments 36 to 38, wherein R represents C₁₋₆alkylene.

Embodiment 41. The compound according to embodiment 40, wherein R represents C₁₋₃alkylene.

Embodiment 42. The compound according to embodiment 41, wherein R represents methylene or propylene.

Embodiment 43. The compound any of embodiments 36 to 42, wherein Z represents

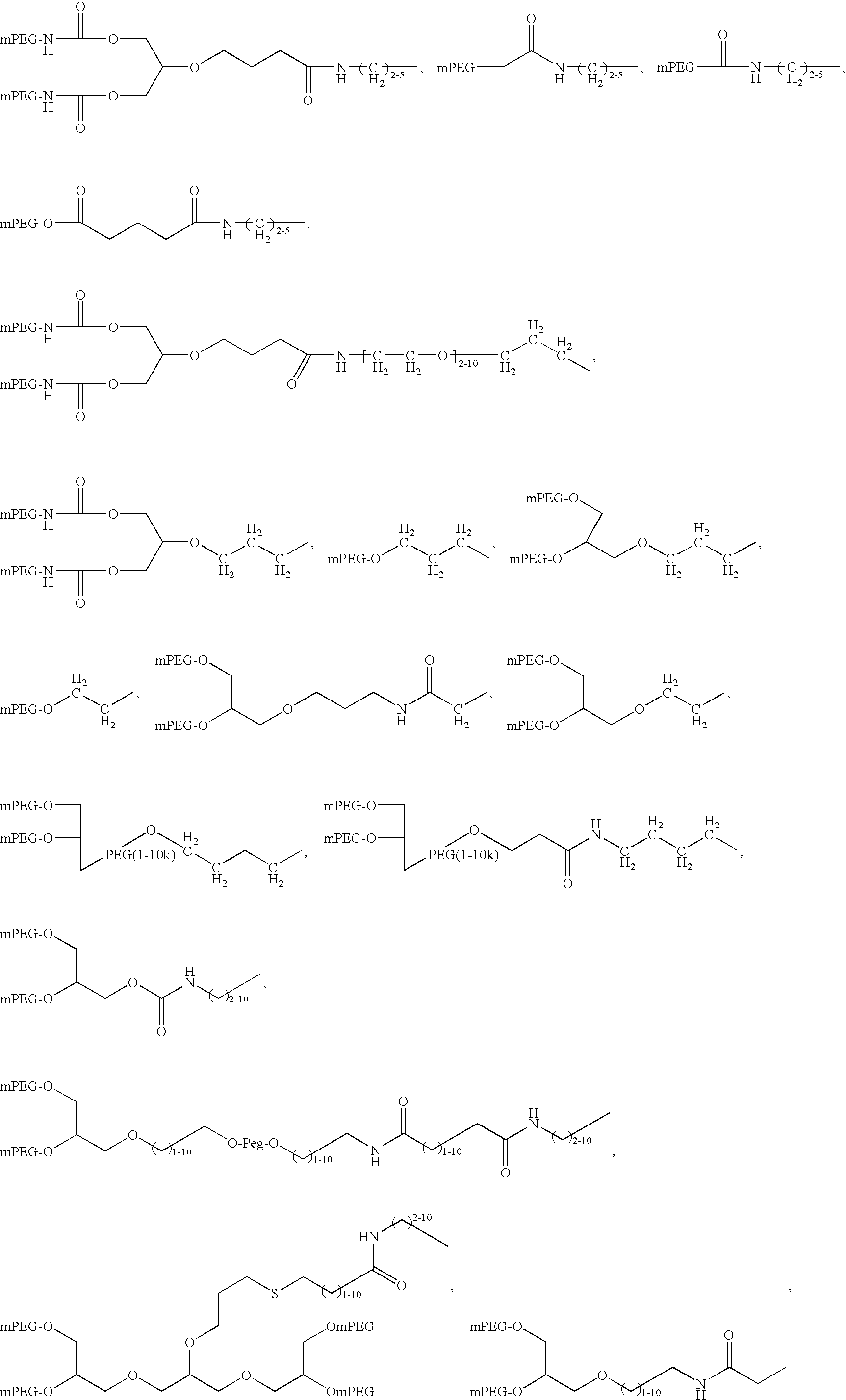

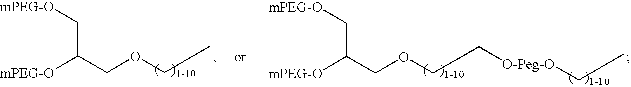

wherein, unless otherwise indicated, mPEG indicates a mPEG with a molecular weight of between 5 kDa and 40 kDa and PEG indicates a PEG with a molecular weight between 1 kDa and 10 kDa;
provided that if Z is

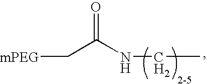

then PEG is 10 kDa PEG.

Embodiment 44. The compound according to any of embodiments 36 to 43, wherein the polypeptide is a glutamine residue comprising growth hormone and PP represents a growth hormone radical obtained by removing —C(═O)—NH₂ from the side chain of two glutamine residues present in the growth hormone.

Embodiment 45. The compound according to embodiment 44, wherein GH represents the radical obtained by removing —C(═O)—NH₂ from the side chain of two glutamine residues present in an hGH comprising the amino acid sequence of SEQ ID No. 1.

Embodiment 46. The compound according to embodiment 44, wherein GH represents the radical obtained by removing —C(═O)—NH₂ from the side chain of two glutamine residues present in 20 kDa hGH.

Embodiment 47. The compound according to embodiment 44, wherein

GH represents the radical obtained by removing —C(═O)—NH₂ from the side chain of a glutamine residue in the position corresponding to position 40 in SEQ ID No. 1, and from the side chain of the glutamine residue in the position corresponding to position 141 in SEQ ID No. 1; or
GH represents the radical obtained by removing —C(═O)—NH₂ from the side chain of a glutamine residue in the position corresponding to position 40 in SEQ ID No. 1, wherein the glutamine residue in the position corresponding to position 141 in SEQ ID No. 1 has been deleted or substituted with another amino acid, and by removing —C(═O)—NH₂ from the side chain of another glutamine residue present in a position different from the positions corresponding to positions 40 and 141 in SEQ ID No. 1; or
GH represents the radical obtained by removing —C(═O)—NH₂ from the side chain of a glutamine residue in the position corresponding to position 141 in SEQ ID No. 1, wherein the glutamine residue in the position corresponding to position 40 in SEQ ID No. 1 has been deleted or substituted with another amino acid, and by removing —C(═O)—NH₂ from the side chain of another glutamine residue present in a position different from the positions corresponding to positions 40 and 141 in SEQ ID No. 1; or
GH represents the radical obtained by removing —C(═O)—NH₂ from the side chain of two glutamines present in an hGH in positions different from the positions corresponding to positions 40 and 141 in SEQ ID No. 1, wherein any glutamine residues present in the positions corresponding to positions 40 and 141 in SEQ ID No. 1 has been deleted or substituted with other amino acids.

Embodiment 48. Human growth hormone, which is covalently attached to a moiety comprising PEG, and in particular mPEG, wherein said PEG comprising moiety is attached to the side chain of glutamine residue 40, to the side chain of glutamine 141 or to the side chains of glutamine 40 and glutamine 141 of human growth hormone, provided it is not
N^{ε141}-[2-(4-(4-(mPEG(20k)ylbutanoyl)-amino-butyloxyimino)-ethyl]hGH,
N^{ε141}-[2-(1-(hexadecanoyl)piperidin-4-yl)ethyloxyimino)-ethyl]hGH,
N^{ε141}(2-(4-(4-(1,3-bis(mPEG(20k)ylaminocarbonyloxy)prop-2-yloxy)butyrylamino)butyloxyimino)ethyl)hGH,
N^{ε141}(2 (4-(2,6-bis(mPEG(20k)yloxycarbonylamino)hexanoylamino)butyloxyimino)ethyl)hGH,
N^{ε141}(2 (4-(4-(mPEG(30k)yloxy)butyrylamino)butyloxyimino)ethyl)hGH,
N^{ε141}(2-(4-(4-(mPEG(20k)yloxy)butyrylamino)butyloxyimino)ethyl)hGH, or
N^{ε141}(2-(4-(3-(mPEG(30k)yloxy)propanoylamino)butyloxyimino)ethyl)hGH.

Embodiment 49. A compound obtained by use of a method according to any of embodiments 1 to 22.

Embodiment 50. A compound obtainable by use of a method according to any of embodiments 1 to 22.

Embodiment 51. A compound obtained by use of a method according to any of embodiments 19 to 22.

Embodiment 52. A compound obtainable by use of a method according to any of embodiments 19 to 22.

Embodiment 53. The compound according to any of embodiments 31 to 35, any of embodiments 44 to 52, embodiment 51, or embodiment 52 for use in therapy.

Embodiment 54. A pharmaceutical composition comprising a compound according to any of embodiments 31 to 35, any of embodiments 44 to 52, embodiment 51, or embodiment 52.

Embodiment 55. A method for treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucucorticoid treatment inchildren; for acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound according to any of embodiments 31 to 35, any of embodiments 44 to 52, embodiment 51, or embodiment 52 or a pharmaceutical composition according to embodiment 54.

Embodiment 56. The use of a compound according to any of embodiments 31 to 35, any of embodiments 44 to 52, embodiment 51, or embodiment 52 in the manufacture of a medicament to be used in the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucucorticoid treatment inchildren; acceleration of the healing of muscle tissue, nervous tissue or wounds; the acceleration or improvement of blood flow to damaged tissue; or the decrease of infection rate in damaged tissue.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law), regardless of any separately provided incorporation of particular documents made elsewhere herein.

All headings and sub-headings are used herein for convenience only and should not be construed as limiting the invention in any way,

Any combination of the above-described elements in all possible variations thereof is encompassed by the invention unless otherwise indicated herein or otherwise clearly contradicted by context.

The use of the terms “a” and “an” and “the” and similar referents in the context of describing the invention are to be construed to cover both the singular and the plural, unless otherwise indicated herein or clearly contradicted by context. For example, the phrase “the compound” is to be understood as referring to various “compounds” of the invention or particular described aspect, unless otherwise indicated.

Recitation of ranges of values herein are merely intended to serve as a shorthand method of referring individually to each separate value falling within the range, unless otherwise indicated herein, and each separate value is incorporated into the specification as if it were individually recited herein.

Unless otherwise indicated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or aspect of the invention using terms such as “comprising”, “having,” “including,” or “containing” with reference to an element or elements is intended to provide support for a similar aspect or aspect of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). Basic and novel properties with respect to such aspects of the invention are provided here.

This invention includes all modifications and equivalents of the subject matter recited in the aspects or claims presented herein to the maximum extent permitted by applicable law.

### EXAMPLES

The TGase used in the examples is microbial transglutaminase from Streptoverticillium mobaraenae according to U.S. Pat. No. 5,156,956 or from Streptomyces lydicus according to WO 9606931-A1.

### Analytical Methods:

### Maldi-T of Mass Spectrometry.

Molecular weights were determined using the Autoflex Maldi-T of instrument (Bruker). Samples were prepared according to the sandwich method. Matrix 1 was a solution of 10 mg alfa-cyano-4-hydroxy-cinnamic acid in 1 ml acetone. Matrix 2 was a solution of 10 mg alfa-cyano-4-hydroxy-cinnamic acid in 1 ml 50% acetonitrile in water. Samples were prepared on the garget by sequentially applying 1 μl matrix 1, air drying, applying 1 μl 3% trifluoracetic acid, applying 1 μl sample, applying 1 μl matrix 2, air drying, washing by flushing the target plate with water and finally air drying. The spectra were acquired using 20% laser power and the standard method for the 3-20 kDa range which was supplied with the instrument.

### Quantification of Protein

Protein concentrations were estimated by measuring absorbance at 280 nm using a UV-spectrofotometer. A molar molar extinction coefficient of 16170 M^{÷1} cm^{÷1} was used. Amounts were calculated from volumes and concentrations.

### SDS Page

SDS poly-acrylamide gel electrophoresis was performed using NuPAGE 4%-12% Bis-Tris gels (Invitrogen NP0321 BOX). The gels were silver stained (Invitrogen LC6100) or Coomassie stained (Invitrogen LC6065) and where relevant also stained for PEG with barium iodide as described in M. M. Kurfurst, Anal.Biochem. 200(2), 244-248 (1992).

### RP-HPLC Analysis.

System A.: Merck-Hitachi system consisting of: L-7400 UV detector, L-7200 autosampler and L-7100 pump. A Zorbax SB-300 4.6 mm×50 mm 5 μm C-18 silica column (Agilent Technologies) was used and detection was by UV at 214 nm. The column was equilibrated with 0.1% trifluoracetic acid/H₂O and eluted by a gradient of 0 to 90% acetonitrile against 0.1% trifluoracetic acid/H₂O over 10 min at ambient temperature, with a flow of 1 ml/min.

### CE (Capillary Electrophoresis) Analysis:

Capillary electrophoresis was carried out using an Agilent Technologies 3DCE system (Agilent Technologies). Data acquisition and signal processing were performed using Agilent Technologies 3DCE ChemStation. The capillary was a 64.5 cm (56.0 cm efficient length) 50 μm i.d. “Extended Light Path Capillary” from Agilent. UV detection was per-formed at 200 nm (16 nm Bw, Reference 380 nm and 50 nm Bw). The running electrolyte was phosphate buffer 50 mM pH7 (method A) or phosphate buffer 50 mM pH2.5 (method B). The capillary was conditioned with 0.1 M NaOH for 3 min, then with Milli-Q water for 2 min and with the electrolyte for 3 min. After each run, the capillary was flushed with milli-Q water for 2 min, then with phosphoric acid for 2 min, and with milli-Q water for 2 min. The hydrodynamic injection was done at 50 mbar for 4.0 s. The voltage was +25 kV. The capillary temperature was 30 C and the runtime was 10.5 min (method A) or 20 min (method B).

Examples 1 and 2 are intended to provide an illustrative example of PEGylation of hGH using transamination with a latent aldehyde—see also WO2005070468.

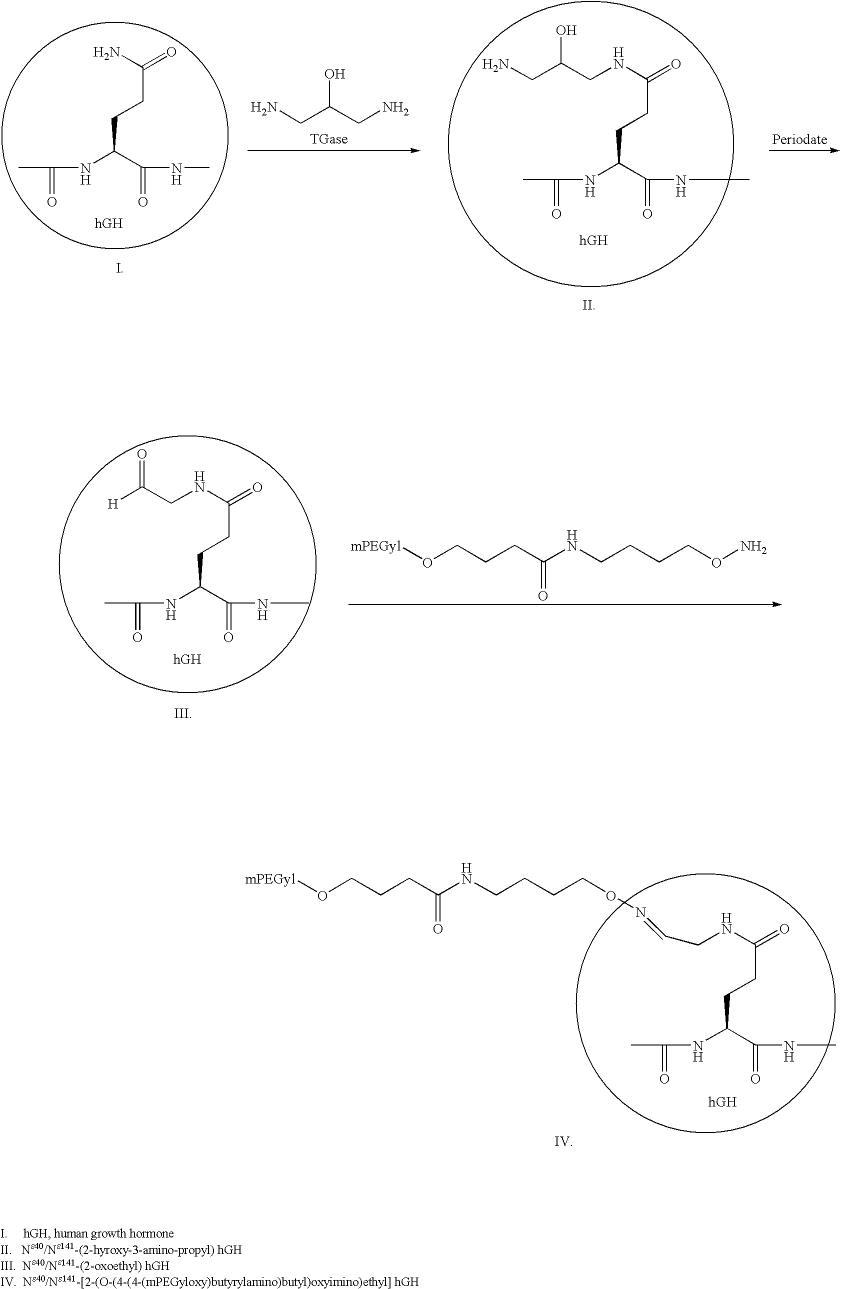

### Example 1

### Preparation of N^{ε141}-[2-O-(4-(4-(1,3-bis(mPEG(20000)aminocarbonyloxy)-2-propyloxy)butyrylamino)butyl)oxyimino)ethyl]

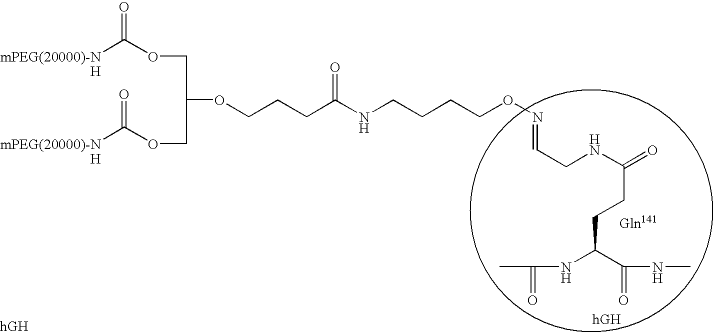

### (a) Trans-amination of hGH (I.) to give N^{ε141}-(2-hydroxy-3-aminopropyl) hGH (II.) hGH (I.) (200 mg) was dissolved in phosphate buffer (50 mM, pH 8.0, 14 ml)

This solution was mixed with a solution of 1,3-Diaminopropan-2-ol (4 mmol, 378 mg) dissolved in phosphate buffer (50 mM, 1 ml, pH 8.0, pH adjusted to 8.0 with dilute hydrochloric acid after dissolution of 1,3-Diamino-propan-2-ol).

Finally a solution of TGase (18 mg˜40 U) dissolved in phosphate buffer (50 mM, pH 8.0, 1 ml) was added and the volume was adjusted to 20 ml by addition of phosphate buffer (50 mM, pH 8.0) giving a concentration of 1,3-Diaminopropan-2-ol at 0.2 M. The combined mixture was incubated for 4 hours at 37° C.

The temperature was lowered to room temperature and N-ethylmaleimide was added to a final concentration of 1 mM.

After further 1 hour the mixture was diluted with 10 volumes of tris buffer (50 mM, pH 8.5).

CE analysis of the resulting mixture shows two major peaks corresponding to hGH and N^{ε141}-(2-hydroxy-3-aminopropyl) hGH (II.) and two minor peaks corresponding to N^{ε40}-(2-hydroxy-3-aminopropyl) hGH (II.) and N^{ε40},N^{ε141}-bis(2-hydroxy-3-aminopropyl) hGH. The last two components are removed during the following purification, but they could have been recovered.

### (b) Ion exchange chromatography of N^{ε141}-(2-hydroxy-3-aminopropyl) hGH (II.)

The solution resulting from (a) was applied to a MonoQ 10/100 GL column (Amersham Biosciences cat. No. 17-5167-01) preequilibrated with buffer A (50 mM tris, pH 8.5). It was then eluted at a flow of 2.5 ml/min with a gradient of 0% to 100% of buffer B (50 mM tris, 0.2 M NaCl, pH 8.5) in buffer A over 63 min. Fractions were collected based on UV absorbtion at 280 nm and Maldi-T of analysis was performed on selected fractions. The fractions corresponding to the largest peak giving the expected mw according to Maldi-T of mass spectrometry were pooled. This pool contains hGH (I.) and N^{ε141}-(2-hydroxy-3-aminopropyl) hGH (II.) in a ratio 60:40 found by CE (method A). Peptide mapping experiments described in International application WO2005DK000028 has previously demonstrated that the combined procedures of (a) and (b) results in selective derivatization at Gln-141.

### (c) Synthesis of N-(4-(tert-Butyloxycarbonylaminoxy)butyl)phthalimide

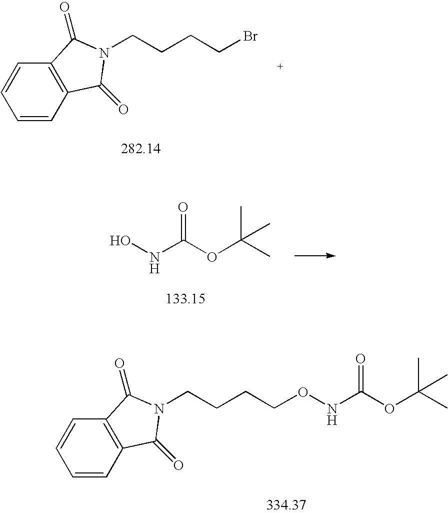

To a stirred mixture of N-(4-bromobutyl)phthalimide (18.9 g, 67.0 mmol), MeCN (14 ml), and N-Boc-hydroxylamine (12.7 g, 95.4 mmol) was added DBU (15.0 ml, 101 mmol) in portions. The resulting mixture was stirred at 50° C. for 24 h. Water (300 ml) and 12 M HCl (10 ml) were added, and the product was extracted three times with AcOEt. The combined extracts were washed with brine, dried (MgSO₄), and concentrated under reduced pressure. The resulting oil (28 g) was purified by chromatography (140 g SiO₂, gradient elution with heptane/AcOEt). 17.9 g (80%) of the title compound was obtained as an oil. ¹H NMR (DMSO-d₆) δ 1.36 (s, 9H), 1.50 (m, 2H), 1.67 (m, 2H), 3.58 (t, J=7 Hz, 2H), 3.68 (t, J=7 Hz, 2H), 7.85 (m, 4H), 9.90 (s, 1H).

### (d) Synthesis of 4-(tert-Butyloxycarbonylaminoxy)butylamine

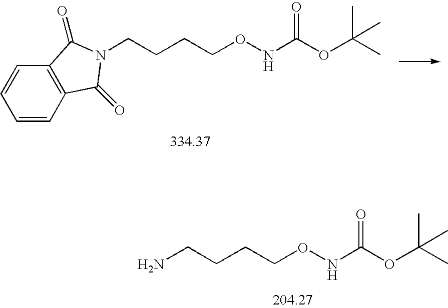

To a solution of N-(4-(tert-butyloxycarbonylaminoxy)butyl)phthalimide obtained from (a) (8.35 g, 25.0 mmol) in EtOH (10 ml) was added hydrazine hydrate (20 ml), and the mixture was stirred at 80° C. for 38 h. The mixture was concentrated and the residue coevaporated with EtOH and PhMe. To the residue was added EtOH (50 ml), and the precipitated phthalhydrazide was filtered off and washed with EtOH (50 ml). Concentration of the combined filtrates yielded 5.08 g of an oil. This oil was mixed with a solution of K₂CO₃ (10 g) in water (20 ml), and the product was extracted with CH₂Cl₂. Drying (MgSO₄) and concentration yielded 2.28 g (45%) of the title compound as an oil, which was used without further purification. ¹H NMR (DMSO-d₆) δ 1.38 (m, 2H), 1.39 (s, 9H), 1.51 (m, 2H), 2.51 (t, J=7 Hz, 2H), 3.66 (t, J=7 Hz, 2H).

### (e) Synthesis of N-Boc-O-(4-(4-(1,3-bis(mPEG(20000)aminocarbonyloxy)-2-propyloxy)butyrylamino)-butyl)hydroxyl-amine

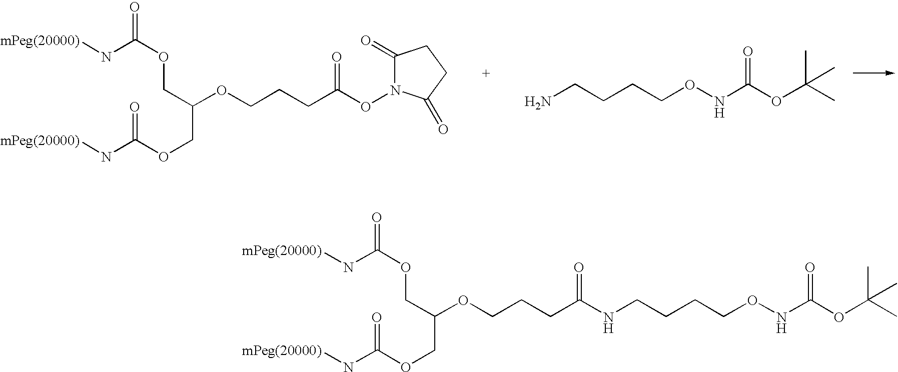

(mPeg(20000)-NH—CO—O—CH₂)₂CH—O—(CH₂)₃—CO—OSu (Nektar, 2Z3Y0T01, 2.0 g, 50 μmol) was mixed with a solution of 4-(Boc-aminoxy)butylamine (187 mg, 915 μmmol) in DCM (12 ml). After stirring at room temperature for 43 h the mixture was added dropwise to stirred Et₂O (200 ml). Filtration, washing with Et₂O. The product was redissolved in DCM (10 ml), and precipitated once more from Et₂O (200 ml). This precipitation was repeated three times. The product was then dissolved in DCM (100 ml) and treated with Amberlyst 15 (11 g; washed with DCM) for 5 min. After filtration and concentration the product was precipitated from Et₂O as above. Filtration and drying under reduced pressure yielded 1.98 g of the title compound as a solid.

### (f) Synthesis of 0-(4-(4-(1,3-bis(mPEG(20000)aminocarbonyloxy)-2-propyloxy)butyrylamino)butyl)-hydroxylamine

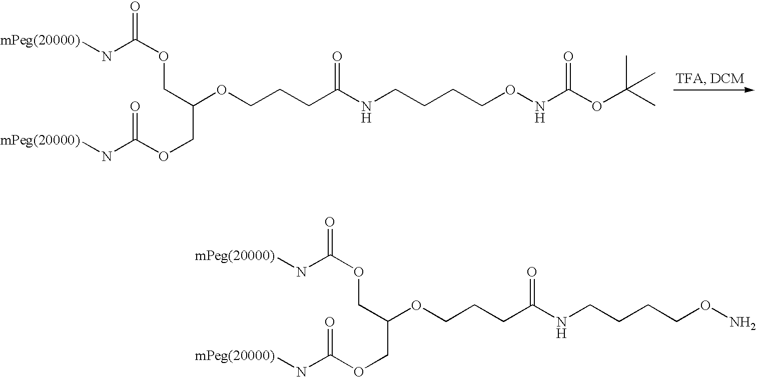

The product from the previous reaction (1.0 g) was dissolved in DCM, and Amberlyst 15 (8.0 g; washed with DCM) was added. After stirring for 10 min the mixture was filtered and the filtrate concentrated. To the residue were added DCM (25 ml) and TFA (25 ml). After standing at room temperature for 0.5 h the mixture was concentrated, the residue coevaporated twice with a mixture of DCM and toluene, and dried under reduced pressure overnight. The residue was dissolved in water (15 ml) and washed twice with water using an Amicon Ultra-15 ultrafiltration device (Millipore). The solution was then neutralized by addition of 2-methylpyridine to pH 6. This solution was used directly for the oximation.

### (g) Oxidation of N^{ε141}-(2-hydroxy-3-aminopropyl) hGH (II.) to give N^{ε141}-(2-oxoethyl) hGH (III.)

The buffer of the pooled fractions from (b) 2 containing 53.8 mg of (I.) and (II.) was exchanged four times to a 15 mM triethanolamine/137 mM 3-(Methylthio)-1-propanol pH 8.5 (adjusted with 1 N hydrochloric acid) buffer using an Amicon Ultra-15 ultrafiltration device (Millipore). Then the solution was concentrated to 5.4 ml. To this was added 0.54 ml of a 25 mM sodiumperiodate in water solution, and the mixture was incubated for 30 min at room temperature in the dark.

The solution was then washed three times with 15 mM triethanolamine buffer pH 8.5 using an Amicon Ultra-15 ultrafiltration device (Millipore). It was then cooled on ice and 1.62 ml ice cold N,N-dimethylformamide was added.

### (h) Oximation of N^{ε141}-(2-oxoethyl) hGH (III.) with O-(4-(4-(1,3-bis(mPEG(20000)oxy)-2-propyloxy)butyrylamino)butyl)hydroxylamine to give N^{ε141}-[2-(O-(4-(4-(1,3-bis(mPEG(20000)oxy)-2-propyloxy)butyrylamino)butyl)oxyimino)ethyl]hGH

The mixture resulting from (g) was slowly added to the PEG solution from (f) under gentle mixing and the reaction was allowed to proceed at room temperature for 72 h.

### (i) Ion exchange chromatography of N^{ε141}-[2-(O-(4-(4-(1,3-bis(mPEG(20000)oxy)-2-propyloxy)butyrylamino)butyl)oxyimino)ethyl]hGH

The solution resulting from (h) was applied to a MonoQ 10/100 GL column (Amersham Biosciences cat. No. 17-5167-01) pre-equilibrated with buffer A (10 mM tris, pH 8.0).

It was then eluted at a flow of 2.0 ml/min with a gradient of 0% to 100% of buffer B (10 mM tris, 0.2 M NaCl, pH 8.0) in buffer A over 79 min. Fractions were collected based on UV absorption at 280 nm. The fractions were pooled corresponding to the wanted peak and concentrated to 10 ml on an Amicon Ultra-15 ultrafiltration device (Millipore).

### (j) Size exclusion chromatography of N^{ε141}-[2-(O-(4-(4-(1,3-bis(mPEG(20000)oxy)-2-propyloxy)butyrylamino)butyl)oxyimino)ethyl]hGH

The concentrated solution resulting from (i) was applied to a HiLoad 26/60 Superdex 200 column (Amersham Biosciences cat. No. 17-1071-01) pre-equilibrated with buffer C (50 mM Ammonium hydrogen carbonate, pH 8.0). It was then eluted at a flow of 0.5 ml/min buffer C over 956 min. Fractions were collected based on UV absorption at 280 nm, and pooled according to earlier tests.

SDS page showed a single band with an apparent molecular weight larger than 120 kDa.

### Example 2

### Preparation of N^{ε141}-[2-(O-(4-(4-(mPEG(30000)oxy)butyrylamino)-butyl)oxyimino)-ethyl]

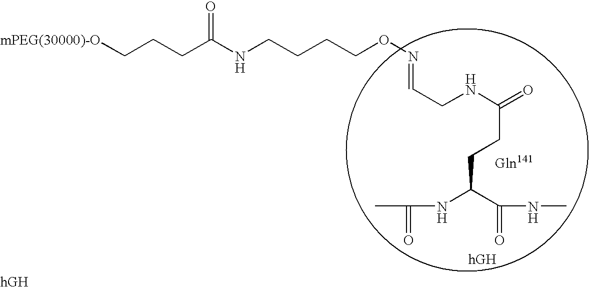

### (a) Trans-amination of hGH (I.) to give N^{ε141}-(2-hydroxy-3-aminopropyl) hGH (II.) hGH (I.) (200 mg) was dissolved in phosphate buffer (50 mM, pH 8.0, 14 ml)

This solution was mixed with a solution of 1,3-Diaminopropan-2-ol (378 mg) dissolved in phosphate buffer (50 mM, 0.5 ml, pH 8.0, pH adjusted to 8.0 with dilute hydrochloric acid after dissolution of 1,3-Diamino-propan-2-ol).

Finally a solution of TGase (18 mg˜40 U) dissolved in phosphate buffer (50 mM, pH 8.0, 0.5 ml) was added and the volume was adjusted to 20 ml by addition of phosphate buffer (50 mM, pH 8.0) giving a concentration of 1,3-Diamino-propan-2-ol at 0.2 M. The combined mixture was incubated for 4 hours at 37° C.

The temperature was lowered to room temperature and N-ethyl-maleimide was added to a final concentration of 1 mM.

After further 1 hour the mixture was diluted with 10 volumes of tris buffer (50 mM, pH 8.5). CE analysis of the resulting mixture shows two major peaks corresponding to hGH and N^{ε141}-(2-hydroxy-3-aminopropyl) hGH (II.) and two minor peaks corresponding to N^{ε40}-(2-hydroxy-3-aminopropyl) hGH (II.) and N^{ε40},N^{ε141}-bis(2-hydroxy-3-aminopropyl) hGH. The last two components are removed during the following purification, but they could have been recovered.

### (b) Ion exchange chromatography of N^{ε141}-(2-hydroxy-3-amino-propyl) hGH (II.)

The solution resulting from (a). was applied to a MonoQ 10/100 GL column (Amersham Biosciences cat. No. 17-5167-01) prequilibrated with buffer A (50 mM tris, pH 8.5). It was then eluted at a flow of 2 ml/min with a step gradient:

Step 1: 0% to 60% buffer B (50 mM tris, 0.2 M NaCl, pH 8.5) in buffer A over 12 min.

Step 2: 60% to 64% buffer B in buffer A over 8 min.

Step 3: 64% buffer B in buffer A for 16 min.

Step 4: 64% to 67% buffer B in buffer A over 16 min.

Step 5: 67% buffer B in buffer A for 16 min.

Step 6: 67% to 100% buffer B in buffer A over 12 min.

Fractions were collected based on UV absorbtion at 280 nm and fractions corresponding to the largest peak were pooled. This pool contains hGH (I.) and N^{ε141}-(2-hydroxy-3-amino-propyl) hGH (II.) in a ratio 58:42 found by CE (method A). Peptide mapping experiments described in International application WO2005DK000028 has previously demonstrated that the combined procedures of (a) and (b) results in selective derivatization at Gln-141.

### (c) Synthesis of O-(4-(4-(mPEG(30000)oxy)butyrylamino)butyl)hydroxylamine

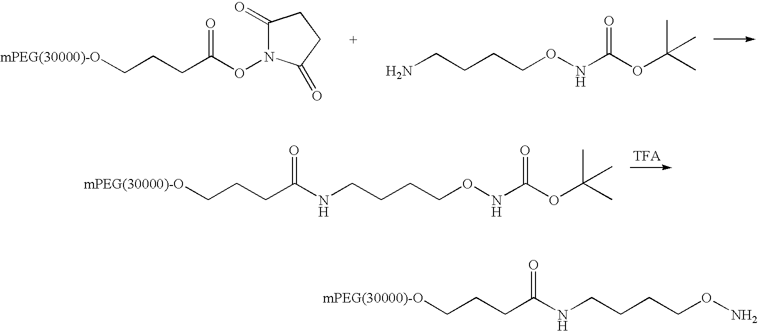

To a solution of 4-(N-Boc-aminoxy)butylamine (0.43 g, 2.10 mmol) in DCM (40 ml) was added mPeg(30000)-O—(CH₂)₃—CO—OSu (Nektar, 2M450R01, MW 30 kDa, 5.0 g, 0.17 mmol). The resulting mixture was stirred at room temperature for 5 d, concentrated under reduced pressure, and the residue was dried in vacuum. Recrystallization from iPrOH (4×80 ml) followed by coevaporation with DCM and drying under reduced pressure yielded 4.14 g of the Boc-protected alkoxylamine.

The product from the previous reaction (0.65 g) was dissolved in 20 ml DCM, and Amberlyst 15 (6.0 g; washed with DCM) was added. After stirring for 10 min the mixture was filtered and the filtrate concentrated. To the residue were added DCM (20 ml) and TFA (20 ml). After standing at room temperature for 0.5 h the mixture was concentrated, the residue coevaporated twice with a mixture of DCM and toluene, and dried under reduced pressure overnight. The residue was dissolved in water (15 ml) and washed twice with water using an Amicon Ultra-15 ultrafiltration device (Millipore). The solution was then neutralized by addition of 2-methylpyridine to pH 6. This solution was used directly for the oximation.

### (d) Oxidation of N^{ε141}-(2-hydroxy-3-aminopropyl) hGH (II.) to give N^{ε141}-(2-oxoethyl) hGH (III.)

The buffer of the pooled fractions from (b) containing 78.14 mg of (1.) and (II.) was exchanged four times to a 15 mM triethanolamine/137 mM 3-(Methylthio)-1-propanol pH 8.5 (adjusted with 1 N hydrochloric acid) buffer using an Amicon Ultra-15 ultrafiltration device (Millipore). Finally the solution was concentrated to 3.3 ml. To this was added 0.33 ml of a 25 mM sodiumperiodate in water was added, and the mixture was incubated for 30 min at room temperature in the dark.

The solution was then washed three times with 15 mM triethanolamine buffer pH 8.5 using an Amicon Ultra-15 ultrafiltration device (Millipore). Then it was cooled on ice and 1.0 ml ice cold N,N-dimethylformamide was added.

### (e) Oximation of N^{ε141}-(2-oxoethyl) hGH (III.) with O-(4-(4-(mPEG(30000)oxy)butyrylamino)butyl)-hydroxylamine to give N^{ε141}-[2-(O-(4-(4-(mPEG(30000)oxy)butyrylamino)butyl)oxyimino)ethyl]hGH

The mixture resulting from (d) was slowly added to the PEG solution from (c) under gentle mixing and the reaction was allowed to proceed at room temperature for 144 h.

### (f) Ion exchange chromatography of N^{ε141}-[2-(O-(4-(4-(mPEG(30000)oxy)butyrylamino)butyl)oxyimino)-ethyl]hGH

The solution resulting from (e) was applied to a MonoQ 10/100 GL column (Amersham Biosciences cat. No. 17-5167-01) pre-equilibrated with buffer A (10 mM tris, pH 8.0). It was then eluted at a flow of 1.5 ml/min with a gradient of 0% to 100% of buffer B (10 mM tris, 0.2 M NaCl, pH 8.0) in buffer A over 107 min. Fractions were collected based on UV absorption at 280 nm. The fractions were pooled corresponding to the wanted peak and concentrated to 10 ml on an Amicon Ultra-15 ultrafiltration device (Millipore).

### (g) Size exclusion chromatography of N^{ε141}-[2-(O-(4-(4-(mPEG(30000)oxy)butyrylamino)-butyl)oxyimino)-ethyl]hGH

The concentrated solution resulting from (f) was applied to a HiLoad 26/60 Superdex 200 column (Amersham Biosciences cat. No. 17-1071-01) pre-equilibrated with buffer C (50 mM Ammonium hydrogen carbonate, pH 8.0). It was then eluted at a flow of 0.5 ml/min buffer C over 956 min. Fractions were collected based on UV absorption at 280 nm, and pooled according to earlier tests.

SDS page showed a single band with an apparent molecular weight of app. 117 kDa.

### Example 3

### Preparation of N^{ε141}-(3-((4(4-(2-(2-(2-(2-(4-(1,3-bis(mPeg(20000)ylaminocarbonyloxy)-2-propyloxy)butyrylamino)ethoxy)ethoxy)ethoxy)ethoxy)butylidene)aminoxy)propyloxy)hGH (VII.)

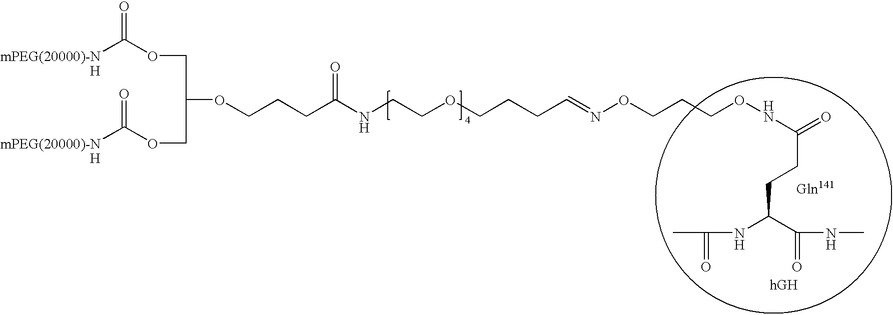

Scheme for the conjugation of hGH with mPEG using transamination with a diaminoxy compound in example 3.

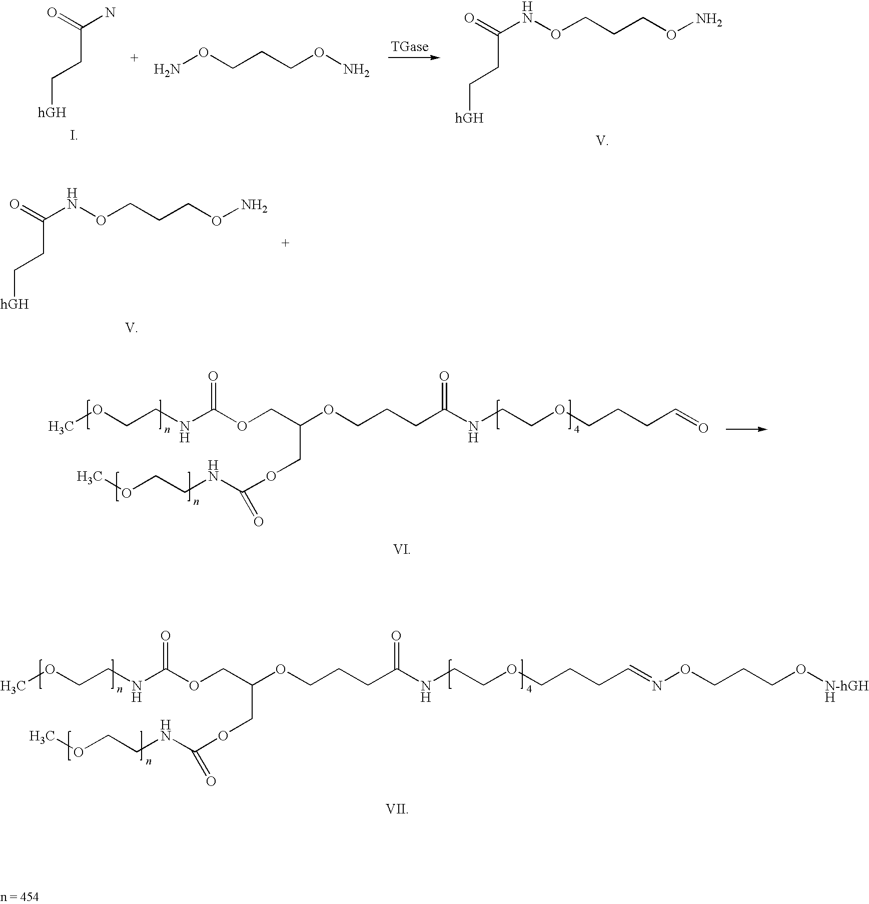

### (a) Trans-amination of hGH (I.) to give N^{ε141}-(3-(aminoxy)propoxy)hGH (V.)

The following solutions were prepared:
1) 100 mg hGH (I.) was dissolved in Na-phosphate buffer (50 mM, pH 6.0) ca. 9 ml. Subsequently, pH was adjusted to 6 and the volume was adjusted to 10 ml using Na-phosphate buffer (50 mM, pH 6.0).
2) 2.00 g 1,3-bisaminoxypropane.2HCl (A. Shirayev, P. K. Thoo lin, and I. K. Moiseev, Synthesis, 38-40 (1997) was dissolved in Na-phosphate buffer (50 mM, pH 6.0) ca. 5 ml, followed by adjustment of pH and volume as for 1.
3) 30 mg Transglutaminase Activa WM (Ajinomoto, 0.3 mg enzyme) was dissolved in Na-phosphate buffer (50 mM, pH 6.0) ca. 9 ml, followed by adjustment of pH and volume as for 1.

Solutions 1+2+3 were mixed, filtered through 0.45 μm filter and allowed to react at 22° C. for a period of 2.5 h. At this point, CE (method B) and Maldi-tof spectroscopy showed approximately 50% conversion of hGH, with approximately 70% of the product being the desired hGH-derivative (V.). To the reaction mixture was added 30 ml of 2 mM aqueous N-ethylmaleimide (NEM). pH was adjusted to 8.0 and the solution was buffer changed 6 times by ultrafiltration (Amicon Ultra 15 (Millipore) 10000 Da cut-off filters, 3600 RCF, room temperature, reduce volume to 1/10) using a 1:1 mixture of aqueous NEM 2 mM and phosphate buffer 50 mM, pH 8.0.

### (b) Oximation of N^{ε141}-(3-(aminoxy)propoxy)hGH (V.) with 4-(2-(2-(2-(2-(4-(1,3-bis(mPeg(20000)ylaminocarbonyloxy)-2-propyloxy)butyrylamino)ethoxy)-ethoxy)ethoxy)ethoxy)butanal (VI.) to give N^{ε141}-(3-((4-(2-(2-(2-(2-(4-(1,3-bis(mPeg(20000)ylaminocarbonyloxy)-2-propyloxy)butyrylamino)ethoxy)ethoxy)ethoxy)ethoxy)butylidene)aminoxy)propyloxy)hGH (VII.)

After last wash in (a), pH was adjusted to 6.0. 180 mg mPEG2-ButyrALD-40K (VI., Nektar 083Y0T01) in 10 ml buffer was added. Reaction was complete within 3 h. RP-HPLC (system A) and SDS-page showed approximately 100% conversion of the hGH-derivative V to the PEGylated hGH (VII.). The reaction buffer changed 3 times with 10 mM TRIS buffer pH 8.5 by ultrafiltration as described above.

### (c) Ion exchange chromatography of N^{ε141}-(3-((4-(2-(2-(2-(2-(4-(1,3-bis(mPeg(20000)ylaminocarbonyloxy)-2-propyloxy)butyrylamino)ethoxy)ethoxy)ethoxy)ethoxy)butylidene)aminoxy)propyloxy)hGH (VII.)

The crude product was purified on a MonoQ 10/100 anion exchange column (Amersham Biosciences) using 10 mM TRIS pH 8.5 (buffer A) and 10 mM TRIS pH 8.5+200 mM NaCl (buffer B) as start and elution buffers, respectively. Elution was performed at 2.0 ml/min with a gradient of 0 to 100% B over a period of 80 min. The product (VII.) eluted in approximately 24% buffer B. Fractions containing the desired product (VII.) were collected, pooled and buffer changed 6 times with 50 mM NH₄HCO₃ buffer pH 8.0, and finally freeze-dried.

The pure product (VII.) was obtained along with two by-products: approx. 20% of an isomer, hGH PEGylated in position Gln-40, and approximately 10% of a dimer, hGH PEGylated in both positions Gln-40 and Gln-141. These products were separable by anion exchange chromatography. The identity of the products were established by peptide mapping experiments and SDS-page.

### Example 4

### Preparation of N^{ε141}-[2-(2-(2,3-bis(mPeg(20000)yloxy)propyloxycarbonylamino)ethyloximino)ethyl]hGH

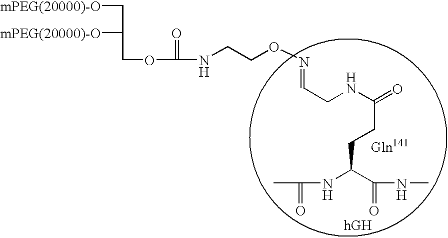

### (a), (b) N^{ε141}-(2-oxoethyl)hGH

N^{ε141}-(2-oxoethyl)hGH (252 mg)was prepared as described in Example 1.

### (c) Oximation of N^{ε141}-(2-oxoethyl)hGH (III.) with P-(2-(2,3-(mPeg(20000)yloxy)propyloxycarbonylamino)ethyl)hydroxylamine (Sunbright GL2-400 CA, NOF Corp., Tokyo, Japan) to give N^{ε141}-[2-(2-(2,3-(mPeg(20000)yloxy)propyloxycarbonylamino)ethyloximino)ethyl]hGH.

The aminoxy-PEG derivative (452 mg Sunbright GL2-400 CA, NOF Corp.) was dissolved in 5.5 ml buffer (2-(N-morpholino)ethanesulphonic acid (MES), 0.3 M, pH 6.5). Then the concentrated pool from the oxidation was diluted with 1.25 ml ice cold NMP and slowly added to the solution of the PEG reagent. The mixture was stirred slowly for 2 days.

### (d) Ion exchange chromatography of N^{ε141}-[2-(2-(2,3-(mPeg(20000)yloxy)propyloxycarbonylamino)ethyloximino)ethyl]hGH

The reaction mixture from (c) was buffer-exchanged into 20 mM triethanolamine buffer pH 8.5 using a desalting column (HiPrep 26/10 Amersham Biosciences cat. No. 17-5087-01) and the protein applied to an ion-exchanger column (HiLoad 26/10 Q Sepharose HP, Ammersham Biosciences) pre-equilibrated with buffer A (20 mM trethanolamine, pH 8.5). It was then eluted at a flow of 5 ml/min with a gradient of 0% to 100% of buffer B (20 mM triethanolamine, 0.2 M NaCl, pH 8.5) in buffer A over 10 column volumes. Fractions were collected based on UV absorption at 280 nm. The fractions corresponding to the desired peak were pooled.

### (e) Size exclusion chromatography of N^{ε141}-[2-(2-(2,3-(mPeg(20000)yloxy)propyloxycarbonylamino)ethyloximino)ethyl]hGH

The pool from (d) was concentrated to 15 ml by ultrafiltration and the protein buffer-exchanged to 50 mM ammonium hydrogen carbonate pH 8.0 using a desalting column (HiPrep 26/10 Amersham Biosciences cat. No. 17-5087-01). The protein was then applied to a size exclusion column (HiLoad 26/10 Superdex 200, Amersham Biosciences cat. No. 17-1071-01) pre-equilibrated with 50 mM Ammonium hydrogen carbonate, pH 8.0). It was then eluted at a flow of 1.0 ml/min. Fractions were collected based on UV absorption at 280 nm.

### Example 5

### Preparation of N^{ε141}-[2-(O-(2-(2-(mPEG(40000)yloxy)ethylamino)-2-oxoethyl)oximino)ethyl]hGH

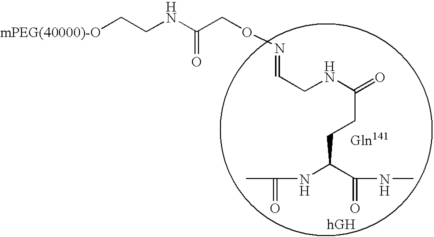

### (a) Transamination.

hGH (I) (100 mg) was transaminated with 1,3-diaminopropan-2-ol and the product was purified using procedures similar to those of example 1 (a) and (b). According to UV spectrofotometry and CE analysis the pool resulting from the purification contained 30 mg protein where 20 mg was N^{ε141}-(2-hydroxy-3-aminopropyl) hGH (II)

### (b) Synthesis of O-(2-(2-(mPEG(40000k)yloxy)ethylamino)-2-oxoethyl)hydroxylamine

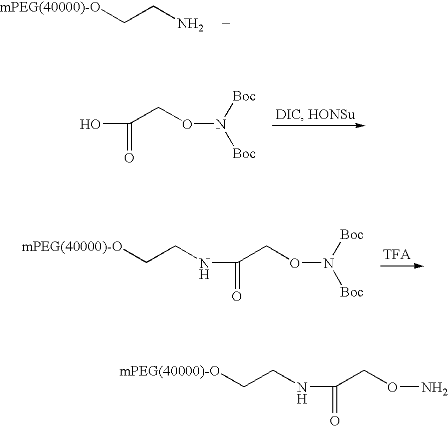

To a solution of Bis-Boc-aminoxyacetic acid (0.30 mg, 0.10 mmol) in DMF (1 ml) was added diisopropylethylamine (0.034 ml, 0.2 mmol), N-hydroxysuccinimide (10 mg, 0.1 mmol) and diisopropylcarbodiimide (0.016 ml, 0.1 mmol). The mixture was stirred for 15 min and mPeg(40000)-O—(CH₂)₂—NH₂ (Product 008-028, Chirotech Ltd. Cambridge UK, MW 40 kDa, 1.0 g, 0.025 mmol) dissolved in a minimal amount of DCM was added. The resulting mixture was stirred at room temperature for 6 h and the product was precipitated and washed with diethylether. The precipitate was redissolved in a mixture of 0.5 ml DMF and 1 ml DCM and precipitated and washed with diethylether. This was repeated once more and after drying 770 mg precipitate was isolated.

200 mg of the dry precipitate was dissolved in 5 ml DCM and 5 ml TFA was added. After 30 min the mixture was concentrated on a rotary evaporator and stripped with 10 ml ethanol twice. The residual oil was redissolved in a mixture of 0.5 ml DMF and 2 ml DCM and precipitated and washed with diethylether. This was repeated once more and the precipitated PEG derivative was dried and dissolved in 1 ml buffer (0.3 M MES, pH 6.5)

The transaminated product obtained in (a) was dissolved in buffer (20 mM triethanolamine, pH 8.5) and 3-(methylthio)-1-propanol (1 ml of a 683 mM solution) was added. Then sodiumperiodate (5 mg, 10 eq.) was added and the mixture was allowed to react 30 min before it was washed three times with aquous methionine solution (168 mM) and concentrated to 4.5 ml using an ultrafiltration device (Amicon Ultra-15, Millipore). Then 0.5 ml ice cold N-methyl-pyrrolidone was added and is slowly added to the solution of the PEG derivative and the mixture is allowed to react overnight.

The buffer of the reaction mixture was exchanged on a desalting column (HiPrep 26/10 Amersham Biosciences cat. No. 17-5087-01) which was pre-equilibrated and eluted with buffer (tris 10 mM, pH 8.5) and the pool was applied to a ion exchange column (HiLoad 26/10 QSepharose, Amersham Biosciences cat. No. 17-1066-01) pre-equilibrated in 10 mM Tris pH 8.5 and eluted with a gradient of 0.2M NaCl in 10 mM Tris ph 8.5 at a flow of 4 ml/min over 10 column volumes. The fractions containing pegylated hGH according to RP-HPLC were pooled and the buffer exchanged on a desalting column (HiPrep 26/10 Amersham Biosciences cat. No. 17-5087-01) which was pre-equilibrated and eluted with buffer (ammonium bicarbonate 50 mM, pH 8.5). The pool was lyophilized. The yield of the target compound was 3.25 mg.

### Example 6

### Preparation of N^{ε141}-[2-(3-(44(1,3-bis(mPeg(30000)ylaminocarbonyloxy)-2-propyloxy)butylidene)aminoxy)propyloxyimino)ethyl]hGH (VII.)

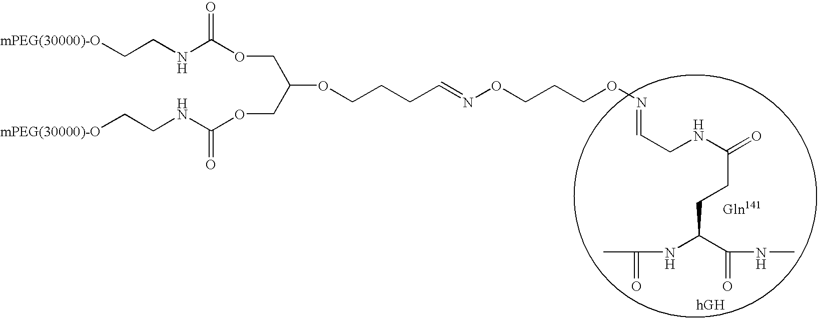

hGH (100 mg) was dissolved in buffer (7 ml, mono-sodium phosphate 125 mM, pH 6.0). 1,3-bis-aminoxypropane.2HCl (2.0 g in 5.0 ml 125 mM mono-sodium phosphate, adjusted to pH 6.0) was added and finally TGase (Activa WM, Ajinomoto, 30 mg in 2 ml mono-sodium phosphate 125 mM, pH 6.0) was added and the volume adjusted to 30 ml with mono-sodium phosphate buffer (125 mM, pH 6.0).

After 4 h at 37° C. the reaction was stopped by addition of NEM (100 ul, 100 mM NEM). After further 1 h at 37° C. the excess bis-aminoxypropane was removed and the buffer exchanged on a desalting column (HiPrep 26/10 Amersham Biosciences cat. No. 17-5087-01) which was pre-equilibrated and eluted with buffer (Tris 10 mM, pH 8.5).

To the protein-containing pool (32 ml containing 82 mg protein) was dropwise added a solution of a 60 kDa 2-arm PEG aldehyde (Nektar, 083Y0V01MPEG2-BUTYRALD-60K, 800 mg in 10 ml water). After incubation at room temperature overnight the buffer was exchanged in four portions on a desalting column (HiPrep 26/10 Amersham Biosciences cat. No. 17-5087-01) to 10 mM Tris pH 8.5. The combined protein containing pool was applied in three portions to a MonoQ 10/100 GL column equilibrated in 10 mM Tris pH 8.5 and eluted with a gradient of 0.2M NaCl in 10 mM Tris ph 8.5 at a flow of 4 ml/min over 20 column volumes. The fractions containing the target compound were collected, buffer-exchanged and re-chromatographed as above. Finally the fractions containing the target compound were pooled and the pool was applied to a HiLoad 26/60 Superdex 200 PG gelfiltration column. (Amersham Biosciences cat. No. 171071-01) which was pre-equilibrated and eluted with 50 mM ammonium bicarbonate. The fractions containing the target compound were pooled and lyophilized. The yield was 35.74 mg of the desired product.

### Pharmacological Methods

Assay (I) BAF hGH-R Assay to Determine In Vitro Growth Hormone Activity

The BAF hGH-R assay is an in vitro proliferation assay, where BAF-3 cells have been modified to be dependent on growth hormone (GH) for growth and survival. BAF-3 is an immortalized murine bone marrow-derived pro-B cell line. Originally, BAF-3 cells are dependent on IL-3 for growth and survival. IL-3 signaling is initiated when one IL-3 molecule binds and dimerizes two IL-3 receptors. This leads to activation of the JAK-2/STAT signaling pathway and thereby regulation of transcription of genes important for growth and survival. The GH-receptor (GH-R) belongs to the same receptor superfamily as the IL-3R (the cytokine/hematopoietin receptor superfamily) and share the same JAK/STAT intracellular signaling pathway. Thus, after transfection of the human GH-R into the BAF-3 cell line, the cell line was turned into a GH-dependent cell line. The cell line shows a dose-related stimulation of growth by adding increasing concentrations of human GH or test compound.

The BAF hGH-R assay is initiated by starving the cells for hGH (culture medium without hGH) for 24 hours at 37° C. and 5% CO₂. The cells are centrifuged, the medium is removed and the cells are re-suspended in starvation medium. 20.000 cells/well are seeded into microtiter plates (96 well NUNC TC microwell 96F SI w/lid NUNCLON D cat. No. 167008). Human GH (in different concentrations) or test-compound (in different concentrations) is added to the cells, and the plates are incubated for 68 hours at 37° C. and 5% CO₂.

The metabolic activity of the cells is measured by AlamarBlue® (BioSource cat no Dal 1025). AlamarBlue is a redox indicator, which is reduced by reactions innate to cellular metabolism and, therefore, provides an indirect measure of viable cell number. AlamarBlue® is added to each well and the cells are incubated for another 4 hours. The absorbance is measured in a fluorescence plate reader using an excitation filter of 544 nM and an emission filter of 590 nM.

The absorbance of the samples is plotted as a function of the concentration of GH/test-compound. From the dose-response curves the potency, expressed by the EC₅₀ value (the amount of GH/test-compound that elicit half of the maximal response), can be calculated. Further, the relative in vitro activity of a test-compound can be described by the ratio-value defined as EC₅₀(compound)/EC₅₀(hGH). A ratio-value above 1 indicates that test-compound is less potent compared to human GH.

Table 1 shows the results for the compounds as described in the examples.

TABLE 1
Relative in vitro potency of different hGH compounds described in
the examples in the BAF hGH-R assay. Values are Mean ± SD

| | Ratio |
|---|---|
| | EC50(compound)/EC50(hGH) |
| Compound from | [Mean ± SD] |
| Example 1 | 10 ± 5 |
| Example 2 | 6 ± 2 |
| Example 3 | 9 ± 1 |
| Example 4 | 10 ± 4 |
| Example 5 | 7 ± 2 |
| Example 6 | 20 ± 6 |

### Pharmacokinetics

The pharmacokinetic of the compounds of the examples was investigated in male

Sprague Dawley rats after intravenous (i.v.) and subcutaneous (s.c.) single dose administration.

Test compounds were diluted to a final concentration of 1 mg/ml in a dilution buffer consisting of: Glycine 20 mg/ml, mannitol 2 mg/ml, NaHCO₃ 2.5 mg/ml, pH adjusted to 8.2.

The test compounds were studied in male Sprague Dawley rats weighing 250 g. The test compounds were administered as a single injection either i.v. in the tail vein or s.c. in the neck with a 25 G needle at a dose of 1 mg/kg body weight.

For each test compound blood sampling was conducted according to the following schedule presented in Table 2.

TABLE 2
Blood sampling schedule for each test compound.

| Animal | | Sampling time (h) | | | | | | | | | | | | |
|---|---|---|---|---|---|---|---|---|---|---|---|---|---|---|
| no. | RoA | Predose | 0.08 | 0.25 | 0.5 | 1 | 2 | 4 | 6 | 8 | 18 | 24 | 48 | 72 |
| 1 | s.c. | | | | | | | X | X | X | | X | X | X |
| 2 | | | | | | | | X | X | X | | X | X | X |
| 3 | | | | X | X | X | X | | | | | | | |
| 4 | | | | X | X | X | X | | | | | | | |
| 5 | | | X | | | | | | | | X | | | |
| 6 | | | X | | | | | | | | X | | | |
| 7 | i.v. | | | | X | X | | | X | X | | X | X | X |
| 8 | | | | | X | X | | | X | X | | X | X | X |
| 9 | | X | X | | | | | | | | X | | | |
| 10 | | X | X | | | | | | | | X | | | |

At each sampling time 0.25 ml blood was drawn from the tail vein using a 25 G needle. The blood was sampled into a EDTA coated test tube and stored on ice until centrifugation at 1200×G for 10 min at 4° C. Plasma was transferred to a Micronic tube and stored at −20° C. until analysis.

Test compound concentrations were determined by a sandwich ELISA using a guinea pig anti-hGH polyclonal antibody as catcher, and biotinylated hGH binding-protein (soluble part of human GH receptor) as detector. The limit of detection of the assay was 0.2 nM.

A non-compartmental pharmacokinetic analysis was performed on mean concentration-time profiles of each test compound using WinNonlin Professional (Pharsight Inc., Mountain View, Calif., USA). The pharmacokinetic parameter estimates of terminal half-life (t_{1/2}) and mean residence time (MRT) are presented in Table 3.

TABLE 3
Half-life (t_{1/2}) and mean residence time (MRT) of hGH
compounds from the examples in Spraque Dawley rats
after single dose i.v. and s.c. administration.

| Compound from | RoA | t_{1/2} (h) | MRT (h) |
|---|---|---|---|
| Example 1 | i.v. | 8.3 | 12.7 |
| Example 2 | i.v. | 10.6 | 6.4 |
| Example 4 | i.v. | 4.7 | 9.6 |
| Example 6 | i.v. | 7.3 | 13.1 |

1. A method for covalently attaching PEG to a polypeptide comprising at least one glutamine residue, said method comprising reacting in one or more steps such glutamine residue comprising polypeptide represented by formula [I] 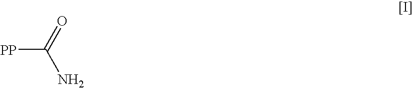
wherein PP represents a polypeptide radical obtained by removing —C(═O)—NH₂ from the side chain of a glutamine residue present in the polypeptide, with a nitrogen containing nucleophile of formula [II]
H₂N-D-R—X [II]
wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated polypeptide of formula [III] 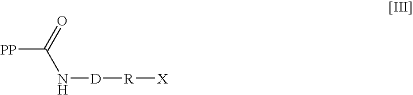
optionally, if X is a latent group, transforming said latent group into —O—NH₂, an aldehyde or a ketone,
said transaminated polypeptide being further reacted with a second compound of formula [IV]
Y—Z [IV]
wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst 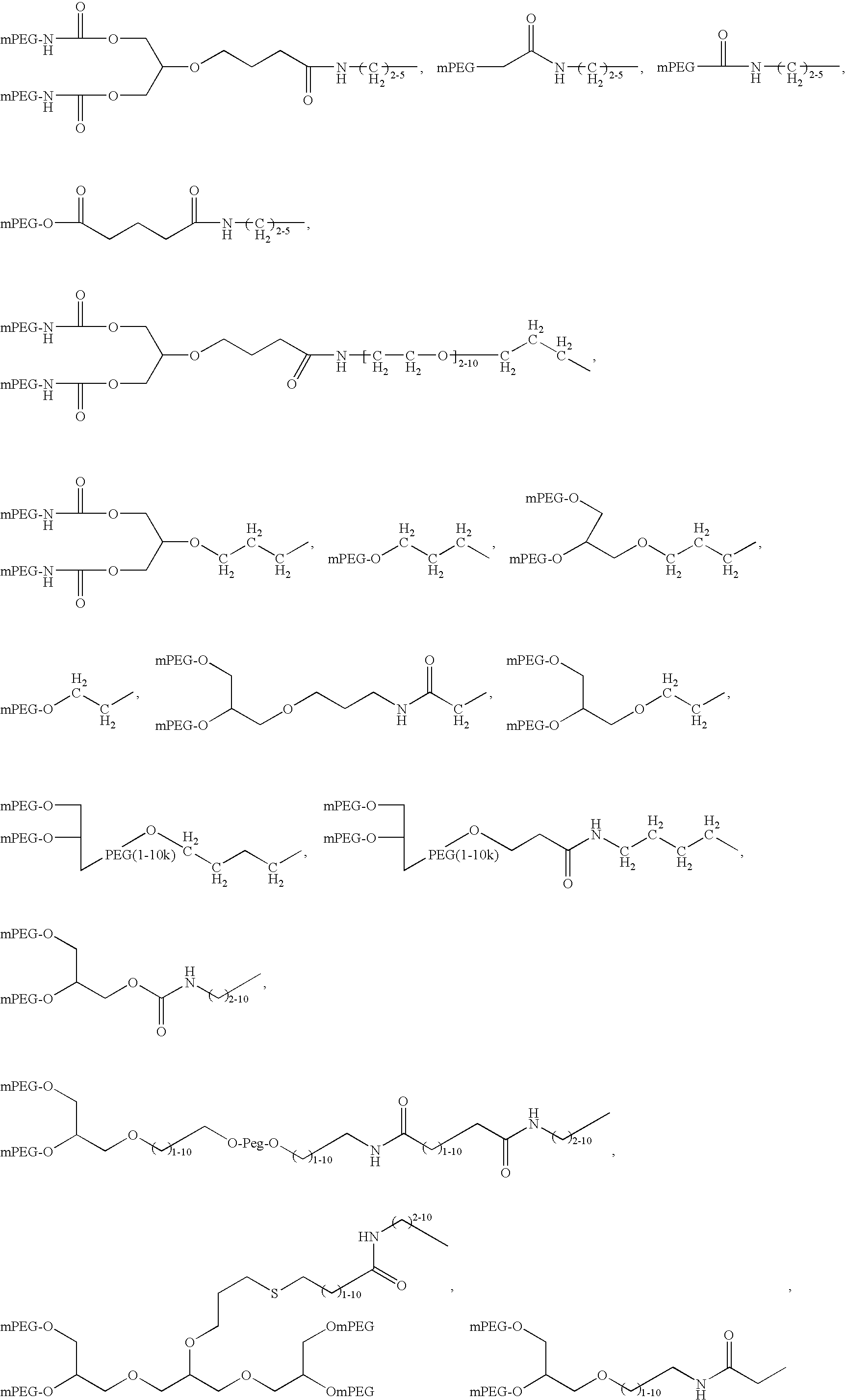 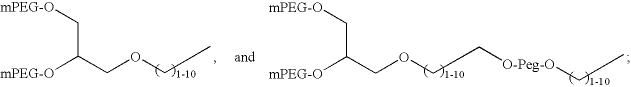
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 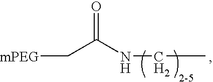
then PEG is 10 kDa PEG
to form a PEGylated polypeptide of formula [V] 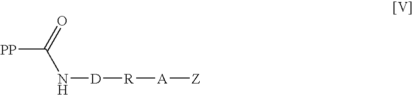
wherein A represents an oxime bond;
or any pharmaceutically acceptable salt, prodrug or solvate thereof.

2. The method according to claim 1, wherein D represents —O—.

3. The method according to claim 1, wherein D represents a single bond.

4. The method according to claim 1, wherein R represents —(CH₂)₄—CH(NH₂)—CO—NH—CH₂— or —(CH₂)₄—CH(NHCOCH₃)—CO—NH—CH₂—.

5. The method according to claim 1, wherein R represents C₁₋₆alkylene.

6. The method according to claim 5, wherein R represents C₁₋₃alkylene.

7. The method according to claim 6, wherein R represents methylene or propylene.

8. The method according to claim 1, wherein Z represents 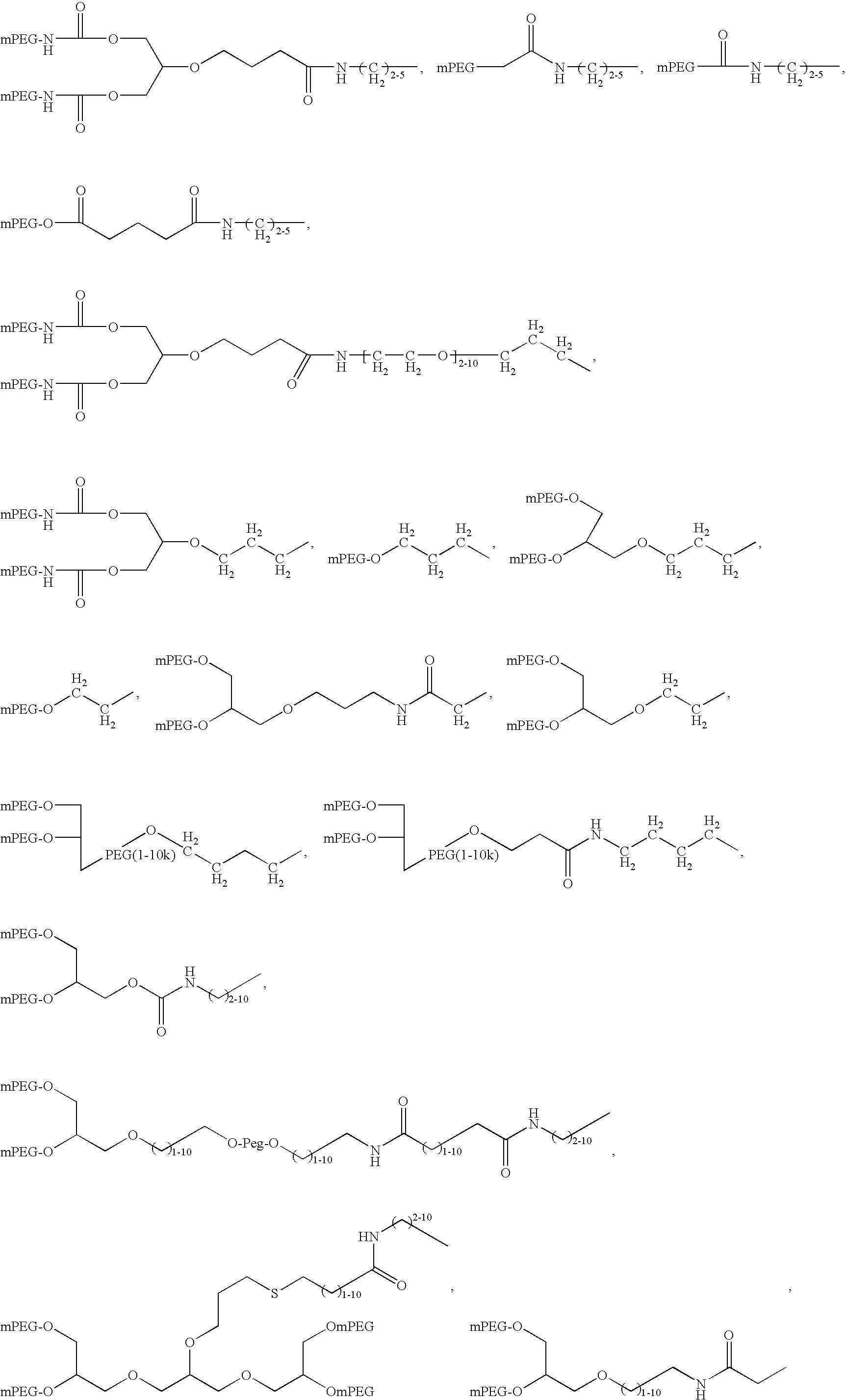 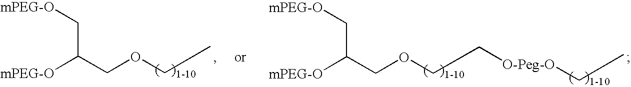
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 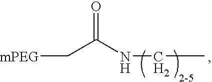
then PEG is 10 kDa PEG.

9. The method according to claim 1, wherein Y represents —O—NH₂ and X represents an aldehyde or a latent group, which may be further reacted to form an aldehyde.

10. The method according to claim 1, wherein Y represents —O—NH₂ and X represents a ketone or a latent group which may be further reacted to form a ketone.

11. The method according to claim 9, wherein the compound of formula [IV]
Y—Z [IV]
represents a compound selected from 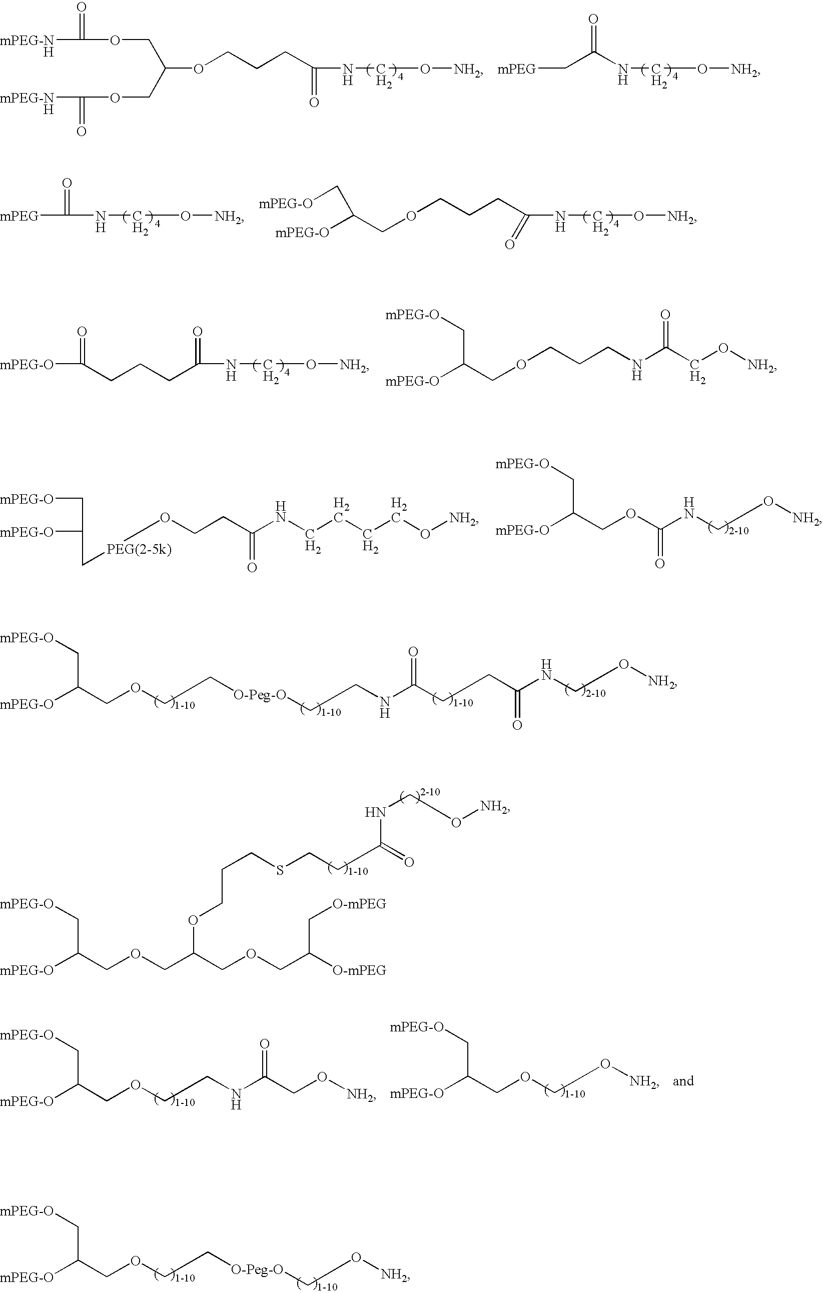
wherein, unless otherwise indicated, mPEG means mPEG with a molecular weight of 10 kDa, 20 kDa or 30 kDa and PEG means PEG with a molecular weight between 2 kDa and 5 kDa.

12. The method according to claim 1, wherein Y represents an aldehyde and X represent —O—NH₂ or a latent group which upon further reaction may be transformed into —O—NH₂.

13. The method according to claim 12, wherein the compound of formula [IV]
Y—Z [IV]
represents a compound selected from 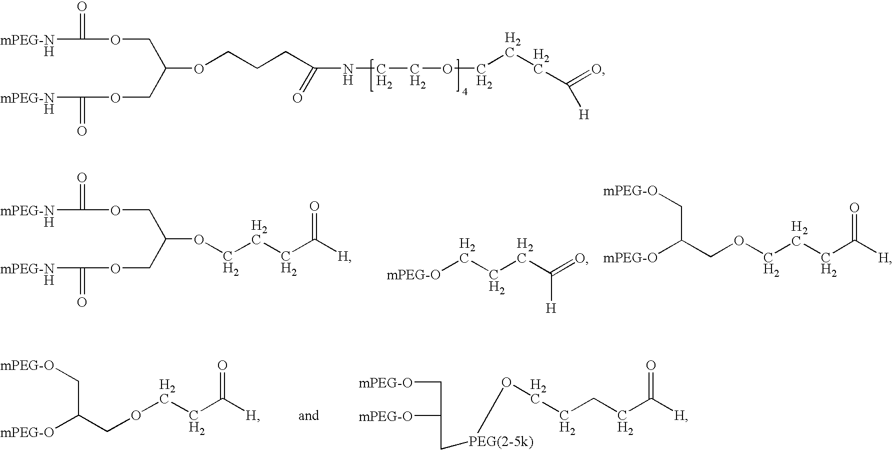
wherein, unless otherwise indicated, mPEG means mPEG with a molecular weight of 10 kDa, 20 kDa or 30 kDa and PEG means PEG with a molecular weight between 2 kDa and 5 kDa.

14. The method according to claim 1, wherein Y represents a ketone and X represent —O—NH₂ or a latent group which upon further reaction may be transformed into —O—NH₂.

15. The method according to claim 14, wherein the compound of formula [IV]
Y—Z [IV]
represents a compound selected from 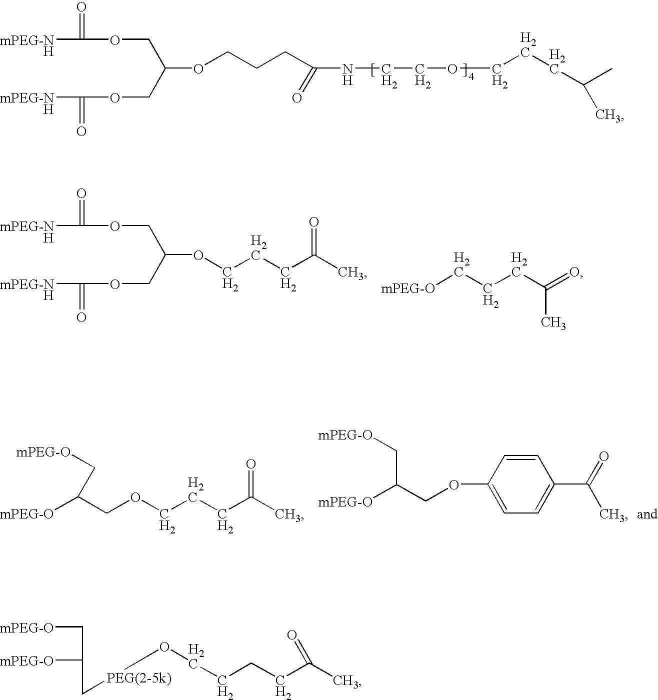
wherein, unless otherwise indicated, mPEG means mPEG with a molecular weight of 10 kDa, 20 kDa or 30 kDa and PEG means PEG with a molecular weight between 2 kDa and 5 kDa.

16. The method according to claim 1, wherein the compound of formula [II]
H₂N-D-R—X [II]
represents 1,3-diamino-2-propanol, and
Y represents —O—NH₂.

17. The method according to claim 1, wherein the compound of formula [II]
H₂N-D-R—X [II]
represents 1,3-diaminooxy propane, and
Y represents an aldehyde.

18. A method of modifying pharmacological properties of a growth hormone, the method comprising covalently attaching PEG to said growth hormone, wherein said growth hormone is represented by formula [I] 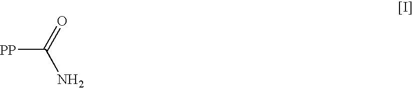
wherein PP represents a polypeptide radical obtained by removing —C(═O)—NH₂ from the side chain of a glutamine residue present in the polypeptide, with a nitrogen containing nucleophile of formula [II]
H₂N-D-R—X [II]
wherein D represents —O— or a single bond;
R represents C₁₋₆alkylene, —(CH₂)₄—CH(NH₂)—CO—NH—CH₂—,
—(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated polypeptide of formula [III] 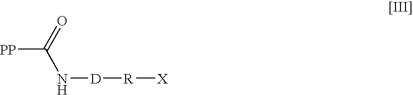
optionally, if X is a latent group, transforming said latent group into —O—NH₂ an aldehyde or a ketone,
said transaminated polypeptide being further reacted with a second compound of formula [IV]
Y—Z [IV]
wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst 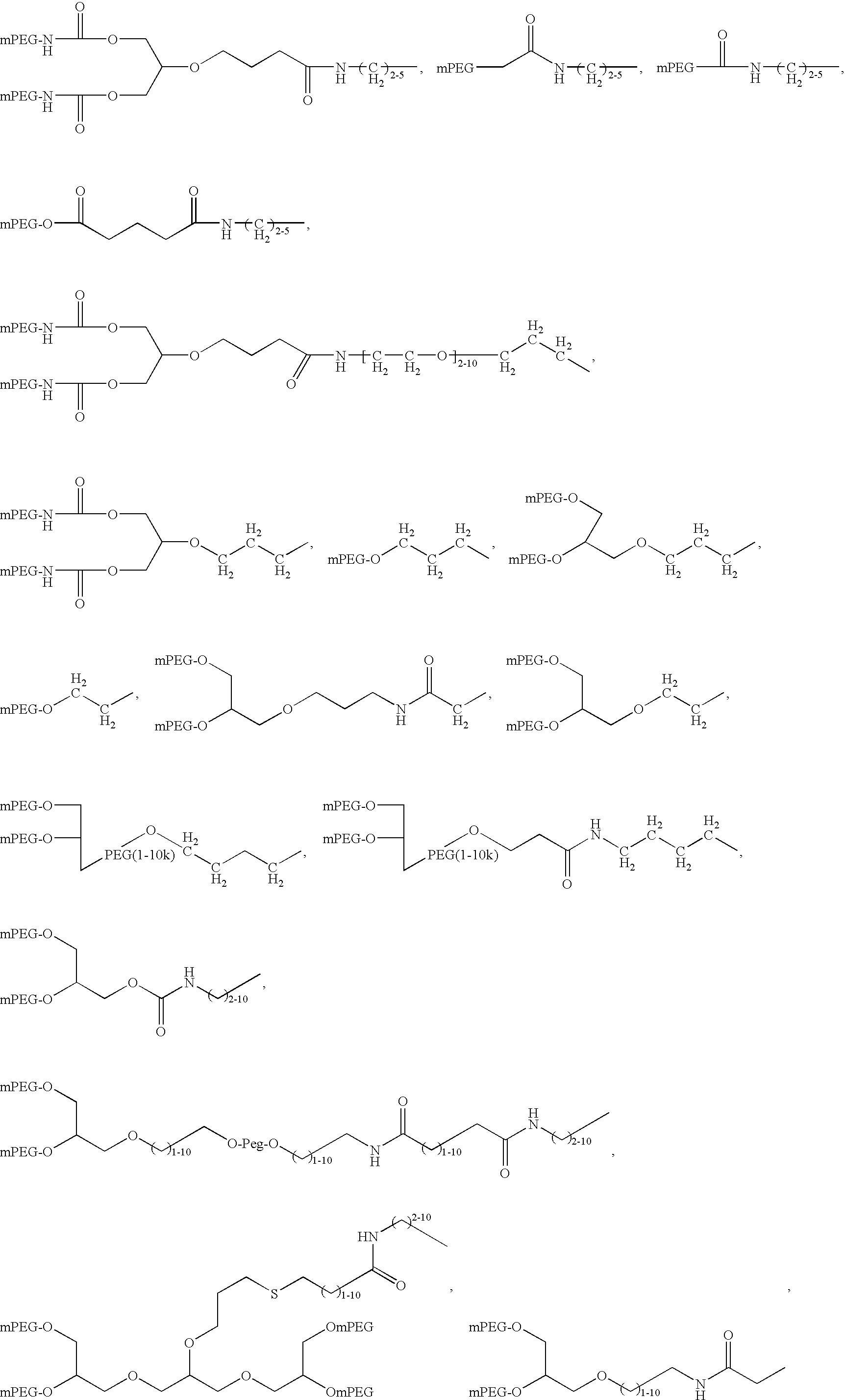 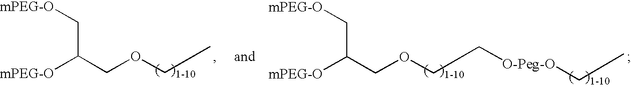
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 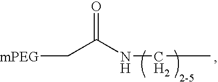
then PEG is 10 kDa PEG to form a PEGylated polypeptide of formula [V] 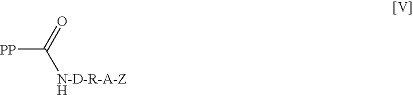
wherein A represents an oxime bond.

19. The method according to according to claim 1, wherein the polypeptide is a glutamine residue comprising growth hormone and PP represents a growth hormone radical obtained by removing —C(═O)—NH₂ from the side chain of a glutamine residue present in growth hormone.

20. The method according to claim 19, wherein said glutamine residue comprising growth hormone represents a human growth hormone.

21. The method according to claim 20, wherein said glutamine residue comprising growth hormone represents
a) a hGH comprising the amino acid sequence of SEQ ID No. 1,
b) 20 kDa hGH,
c) a hGH in which the glutamine residue in the position corresponding to position 40 in SEQ ID No. 1 has been deleted or substituted with another amino acid,
d) a hGH in which the glutamine residue in the position corresponding to position 141 in SEQ ID No. 1 has been deleted or substituted with another amino acid, or
e) a hGH in which the glutamine residue in the position corresponding to position 40 in SEQ ID No. 1, and the glutamine residue in the position corresponding to position 141 in SEQ ID No. 1 each have been deleted or substituted with another amino acid, and where a glutamine residue is present in another position in the growth hormone.

22. The method according to claim 21, wherein said growth hormone represents hGH.

23. A compound according to formula [V] 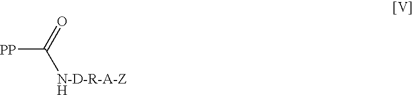
wherein PP represents a polypeptide radical obtained by removing —C(═O)—NH₂ from the side chain of a glutamine residue present in the polypeptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂— or C₅₋₁₅heteroalkylene;
A represents an oxime bond;
Z represents a moiety selected amongst 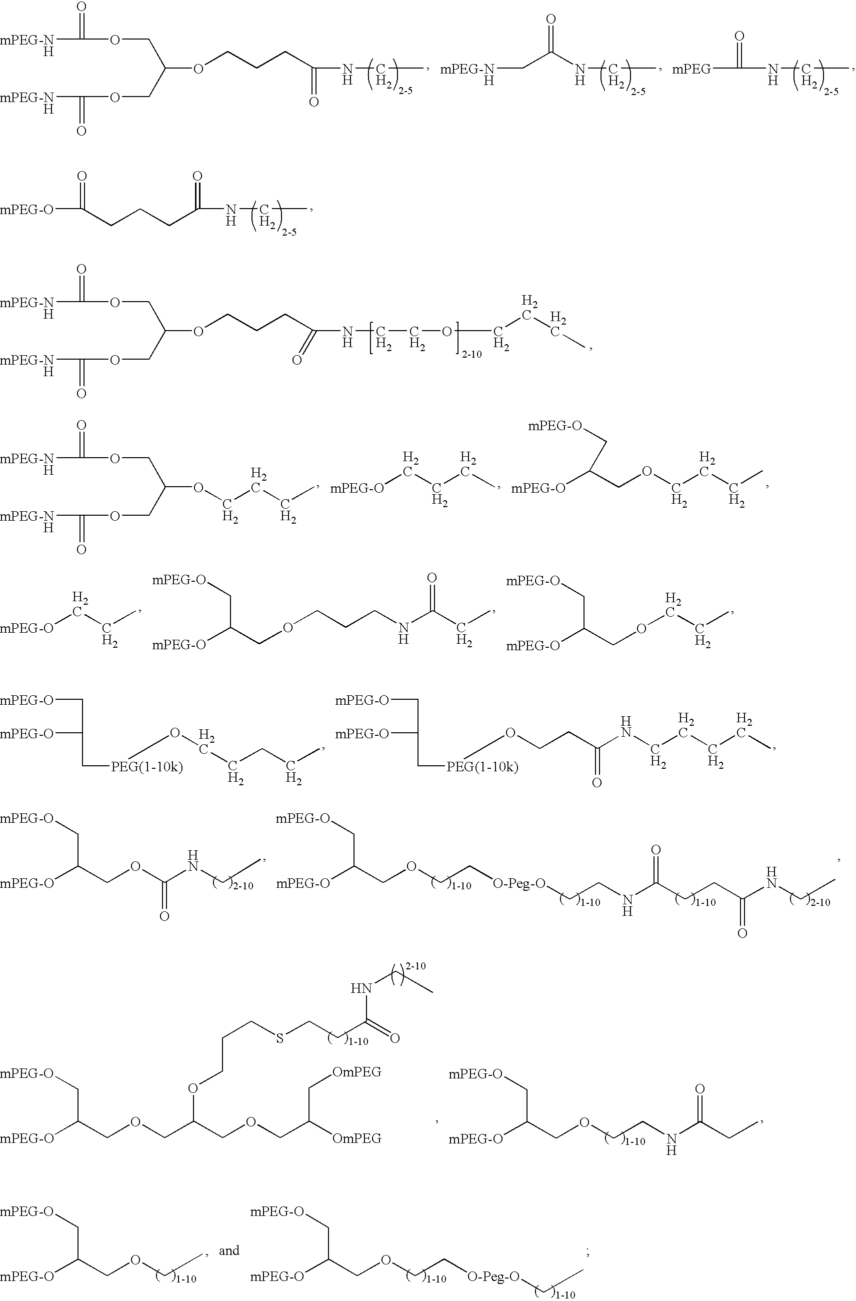
wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof;
provided that if Z is 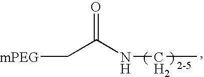
then mPEG is 10 kDa mPEG.

24. The compound according to claim 23, wherein D represents —O—.

25. The compound according to claim 23, wherein D represents a single bond.

26. The compound according to claim 23, wherein R represents —(CH₂)₄—CH(NH₂)—CO—NH—CH₂— or —(CH₂)₄—CH(NHCOCH₃)—CO—NH—CH₂—.

27. The compound according to claim 23, wherein R represents C₁₋₆alkylene.

28. The compound according to claim 27, wherein R represents C₁₋₃alkylene.

29. The compound according to claim 28, wherein R represents methylene or propylene.

30. The compound according to claim 23, wherein Z represents 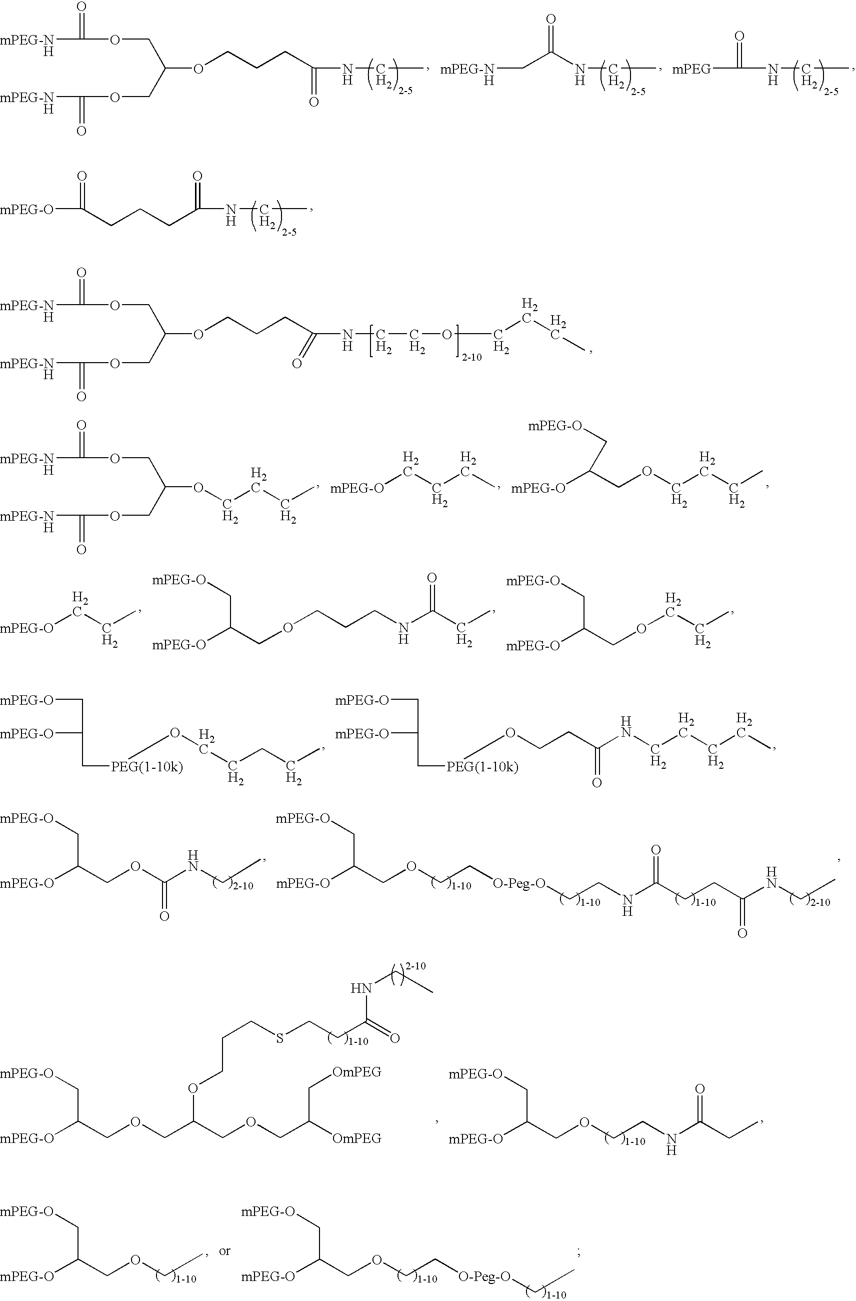
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 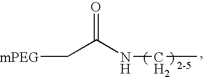
then PEG is 10 kDa PEG.

31. The compound according to claim 23, wherein the polypeptide is a glutamine residue comprising growth hormone and PP represents a growth hormone radical obtained by removing —C(═O)—NH₂ from the side chain of a glutamine residue present in growth hormone.

32. The compound according to claim 31, wherein GH represents the radical obtained by removing —C(═O)—NH₂ from the side chain of a glutamine residue present in an hGH comprising the amino acid sequence of SEQ ID No. 1.

33. The compound according to claim 31, wherein GH represents the radical obtained by removing —C(═O)—NH₂ from the side chain of a glutamine residue present in 20 kDa hGH.

34. The compound according to claim 31, wherein GH represents the radical obtained by removing —C(═O)—NH₂ from the side chain of
a) the glutamine residue in the position corresponding to position 40 in SEQ ID No. 1; or
b) the glutamine residue in the position corresponding to position 141 in SEQ ID No. 1; or
GH represents the radical obtained by removing C(═O)—NH₂ from the side chain of a glutamine residue in the position corresponding to position 40 in SEQ ID No. 1, wherein the glutamine residue in the position corresponding to position 141 in SEQ ID No. 1 has been deleted or substituted with another amino acid; or
GH represents the radical obtained by removing —C(═O)—NH₂ from the side chain of a glutamine residue in the position corresponding to position 141 in SEQ ID No. 1, wherein the glutamine residue in the position corresponding to position 40 in SEQ ID No. 1 has been deleted or substituted with another amino acid; or
GH represents the radical obtained by removing —C(═O)—NH₂ from the side chain of a glutamine residue present in a position different from the position corresponding to position 40 in SEQ ID No. 1 and different from the position corresponding to position 141 in SEQ ID No. 1, wherein the glutamine residue in the position corresponding to position 40 in SEQ ID No. 1, and the glutamine residue in the position corresponding to position 141 in SEQ ID No. 1 each have been deleted or substituted with another amino acid.

35. A compound according to claim 23 selected from
N^{δ141/40}-2-(O-(4-{4-(1,3-bis(mPEG(10k)ylaminocarbonyloxy)prop-2-yloxy)butyryl}aminobutyl)-oximino)ethyl hGH;
N^{δ141/40}-2-(O-(4-{4-(mPEG(10k)yloxy)butyryl}aminobutyl)oximino)ethyl hGH;
N^{δ141/40}-2-(O-(4-{3-(mPEG(10k)yloxy)propionyl}aminobutyl)oximino)ethyl hGH;
N^{δ141/40}-2-(O-(4-{4-(2,3-bis(mPEG(10k)yloxy)prop-1-yloxy)butyryl}aminobutyl)oximino)ethyl hGH;
N^{δ141/40}-2-(O-(4-{5-(mPEG(10k)yloxy-5-oxopentanoyl}aminobutyl)oximino)ethyl hGH;
N^{δ141/40}-3-({-4-(2-(2-(2-(2-(2-(4-(1,3-bis(mPEG(10k)ylamino carbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(10k)ylaminocarbonyloxy)prop-2-yloxy)butylidene}aminoxy)propyloxy hGH;
N^{δ141/40}-3-({4-(mPEG(10k)yloxy)butylidene}aminoxy)propyloxy hGH;
N^{δ141/40}-3-({4-(2,3-bis(mPEG(10k)yloxy)prop-1-yloxy)butylidene}aminoxy)propyloxy hGH;
N^{δ141/40}-3-({3 (mPEG(10k)yloxy)propylidene}aminoxy)propyloxy hGH;
N^{δ141/40}-2-(O-(2-(3-(2,3-bis(mPEG(10k)yloxy)propyloxy)propylamino)-2-oxoethyl)oximino)ethyl hGH;
N^{δ141/40}-3-({4-(2,3-bis(mPEG(10k)yloxy)prop-1-yloxy)propylidene}aminoxy)propyloxy hGH;
N^{δ141/40}-2-(O-(4-{4-(1,3-bis(mPEG(20k)ylaminocarbonyloxy)prop-2-yloxy)butyryl}aminobutyl)-oximino)ethyl hGH;
N^{δ141/40}-2-(O-(4-{4-(2,3-bis(mPEG(20k)yloxy)propionyl}aminobutyl)oximino)ethyl hGH;
N^{δ141/40}-2-(O-(4-{4-(2,3-bis(mPEG(20k)yloxy)prop-1-yloxy)butyryl}aminobutyl)oximino)ethyl hGH;
N^{δ141/40}-2-(O-(4-{5-(mPEG(20k)yloxy-5-oxopentanoyl}aminobutyl)oximino)ethyl hGH;
N^{δ141/40}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(20k)ylaminocarbonyloxy)prop-2-yloxy)butylidene}aminoxy)propyloxy hGH;
N^{δ141/40}-3-({4-(mPEG(20k)yloxy)butylidene}aminoxy)propyloxy hGH;
N^{δ141/40}-3-({4-(2,3-bis(mPEG(20k)yloxy)prop-1-yloxy)butylidene}aminoxy)propyloxy hGH;
N^{δ141/40}-3-({3 (mPEG(20k)yloxy)propylidene}aminoxy)propyloxy hGH;
N^{δ141/40}-2-(O-(2-(3-(2,3-bis(mPEG(20k)yloxy)propyloxy)propylamino)-2-oxoethyl)oximino)ethyl hGH;
N^{δ141/40}-3-({4-(2,3-bis(mPEG(20k)yloxy)prop-1-yloxy)propylidene}aminoxy)propyloxy hGH;
N^{δ141/40}-2-(O-(4-{4-(1,3-bis(mPEG(30k)ylaminocarbonyloxy)prop-2-yloxy)butyryl}aminobutyl)-oximino)ethyl hGH;
N^{δ141/40}-2-(O-(4-{3-(mPEG(30k)yloxyl)propionyl}aminobutyl)oximino)ethyl hGH;
N^{δ141/40}-2-(O-(4-{4-(2,3-bis(mPEG(30k)yloxy)prop-1-yloxy)butyryl}aminobutyl)oximino)ethyl hGH;
N^{δ141/40}-2-(O-(4-{5-(mPEG(30k)yloxy-5-oxopentanoyl}aminobutyl)oximino)ethyl hGH;
N^{δ141/40}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ141/40}-3-({4-(1,3-bis(mPEG(30k)ylaminocarbonyloxy)prop-2-yloxy)butylidene}aminoxy)propyloxy hGH;
N^{δ141/40}-3-({4-(mPEG(30k)yloxy)butylidene}aminoxy)propyloxy hGH;
N^{δ141/40}-3-({4-(2,3-bis(mPEG(30k)yloxy)prop-1-yloxy)butylidene}aminoxy)propyloxy hGH;
N^{δ141/40}-3-({3 (mPEG(30k)yloxy)propylidene}aminoxy)propyloxy hGH;
N^{δ141/40}-2-(O-(2-(3-(2,3-bis(mPEG(30k)yloxy)propyloxy)propylamino)-2-oxoethyl)oximino)ethyl hGH;
N^{δ141/40}-3-({4-(2,3-bis(mPEG(30k)yloxy)prop-1-yloxy)propylidene}aminoxy)propyloxy hGH;
N^{δ141/40}-3-({4-{(2,3-bis(mPEG(20k)yl)prop-1-yloxy)PEGyloxy}butylidene}aminoxy)propyloxy hGH;
N^{δ141/40}-2-((4-(4-((2,3-bis(mPEG(20k)yl)propyl)PEGyloxy)butyrylamino)butyl)oximino)ethyl hGH;
N^{δ141}-2-(O-(4-{-4-(1,3-bis(mPEG(10k)ylaminocarbonyloxy)prop-2-yloxy)butyryl}-aminobutyl)oximino)ethyl hGH;
N^{δ141}-2-(O-(4-{4-(mPEG(10k)yloxy)butyryl}aminobutyl)oximino)ethyl hGH;
N^{δ141}-2-(O-(4-{3-(mPEG(10k)yloxy)propionyl}aminobutyl)oximino)ethyl hGH;
N^{δ141}-2-(O-(4-{4-(2,3-bis(mPEG(10k)yloxy)prop-1-yloxy)butyryl}aminobutyl)oximino)ethyl hGH;
N^{δ141}-2-(O-(4-{5-(mPEG(10k)yloxy-5-oxopentanoyl}aminobutyl)oximino)ethyl hGH;
N^{δ141}-3-({4-(2-(2-(2-(2-(4-(1,3-bis(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(10k)ylamino carbonyloxy)prop-2-yloxy)butylidene}-aminoxy)propyloxy hGH;
N^{δ141}-3-({4-(mPEG(10k)yloxy)butylidene}aminoxy)propyloxy hGH;
N^{δ141}-3-({4-(2,3-bis(mPEG(10k)yloxy)prop-1-yloxy)butylidene}aminoxy)propyloxy hGH;
N^{δ141}-3-({3-(mPEG(10k)yloxy)propylidene}aminoxy)propyloxy hGH;
N^{δ141}-2-(O-(2-(3-(2,3-bis(mPEG(10k)yloxy)propyloxy)propylamino)-2-oxoethyl)oximino)ethyl hGH;
N^{δ141}-3-({4-(2,3-bis(mPEG(10k)yloxy)prop-1-yloxy)propylidene}aminoxy)propyloxy hGH;
N^{δ141}-2-(O-(4-{4-(1,3-bis(mPEG(20k)ylaminocarbonyloxy)prop-2-yloxy)butyryl}aminobutyl)oximino)ethyl hGH;
N^{δ141}-2-(O-(4-{3-(mPEG(20k)yloxy)propionyl}aminobutyl)oximino)ethyl hGH;
N^{δ141}-2-(O-(4-{4-(2,3-bis(mPEG(20k)yloxy)prop-1-yloxy)butyryl}aminobutyl)oximino)ethyl hGH;
N^{δ141}-2-(O-(4-{5-(mPEG(20k)yloxy-5-oxopentanoyl}aminobutyl)oximino)ethyl hGH;
N^{δ141}-3-({4-(2-(2-(2-(2-(4-(1,3-bis(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(20k)ylaminocarbonyloxy)prop-2-yloxy)butylidene}-aminoxy)propyloxy hGH;
N^{δ141}-3-({4-(mPEG(20k)yloxy)butylidene}aminoxy)propyloxy hGH;
N^{δ141}-3-({4-(2,3-bis(mPEG(20k)yloxy)prop-1-yloxy)butylidene}aminoxy)propyloxy hGH;
N^{δ141}-3-({3-(mPEG(20k)yloxy)propylidene}aminoxy)propyloxy hGH;
N^{δ141}-2-(O-(2-(3-(2,3-bis(mPEG(20k)yloxy)propyloxy)propylamino)-2-oxoethyl)oximino)ethyl hGH;
N^{δ141}-3-({4-(2,3-bis(mPEG(20k)yloxy)prop-1-yloxy)propylidene}aminoxy)propyloxy hGH;
N^{δ141}-2-(O-(4-{4-(1,3-bis(mPEG(30k)ylaminocarbonyloxy)prop-2-yloxy)butyryl}aminobutyl)oximino)ethyl hGH;
N^{δ141}-2-(O-(4-{3-(mPEG(30k)yloxy)propionyl}aminobutyl)oximino)ethyl hGH;
N^{δ141}-2-(O-(4-{4-(2,3-bis(mPEG(30k)yloxy)prop-1-yloxy)butyryl}aminobutyl)oximino)ethyl hGH;
N^{δ141}-2-(O-(4-{5-(mPEG(30k)yloxy-5-oxopentanoyl}aminobutyl)oximino)ethyl hGH;
N^{δ141}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ141}-3-({4-(1,3-bis(mPEG(30k)ylaminocarbonyloxy)prop-2-yloxy)butylidene}-aminoxy)propyloxy hGH;
N^{δ141}-3-({4-(mPEG(30k)yloxy)butylidene}aminoxy)propyloxy hGH;
N^{δ141}-3-({4-(2,3-bis(mPEG(30k)yloxy)prop-1-yloxy)butylidene}aminoxy)propyloxy hGH;
N^{δ141}-3-({3-(mPEG(30k)yloxy)propylidene}aminoxy)propyloxy hGH;
N^{δ141}-2-(O-(2-(3-(2,3-bis(mPEG(30k)yloxy)propyloxy)propylamino)-2-oxoethyl)oximino)ethyl hGH;
N^{δ141}-3-({4-(2,3-bis(mPEG(30k)yloxy)prop-1-yloxy)propylidene}aminoxy)propyloxy hGH;
N^{δ141}-3-({4-{(2,3-bis(mPEG(20k)yloxy)prop-1-yl)PEGyloxy}butylidene}aminoxy)propyloxy hGH;
N^{δ141}-2-((4-(4-((2,3-bis(mPEG(20k)yl)propyl)PEGyloxy)butyrylamino)butyl)oximino)ethyl hGH;
N^{δ40}-2-(O-(4-{4-(1,3-bis(mPEG(10k)ylaminocarbonyloxy)prop-2-yloxy)butyryl}-aminobutyl)oximino)ethyl hGH;
N^{δ40}-2-(O-(4-{4-(mPEG(10k)yloxy)butyryl}aminobutyl)oximino)ethyl hGH;
N^{δ40}-2-(O-(4-{3-(mPEG(10k)yloxy)propionyl}aminobutyl)oximino)ethyl hGH;
N^{δ40}-2-(O-(4-{4-(2,3-bis(mPEG(10k)yloxy)prop-1-yloxy)butyryl}aminobutyl)oximino)ethyl hGH;
N^{δ40}-2-(O-(4-{5-(mPEG(10k)yloxy-5-oxopentanoyl}aminobutyl)oximino)ethyl hGH;
N^{δ40}-3-({4-(2-(2-(2-(2-(4-(1,3-bi s(mPEG(10k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(10k)ylaminocarbonyloxy)prop-2-yloxy)butylidene}aminoxy)propyloxy hGH;
N^{δ40}-3-({4-(mPEG(10k)yloxy)butylidene}aminoxy)propyloxy hGH;
N^{δ40}-3-({4-(2,3-bis(mPEG(10k)yloxy)prop-1-yloxy)butylidene}aminoxy)propyloxy hGH;
N^{δ40}-3-({3-(mPEG(10k)yloxy)propylidene}aminoxy)propyloxy hGH;
N^{δ40}-2-(O-(2-(3-(2,3-bis(mPEG(10k)yloxy)propyloxy)propylamino)-2-oxoethyl)oximino)ethyl hGH;
N^{δ40}-3-({4-(2,3-bis(mPEG(10k)yloxy)prop-1-yloxy)propylidene}aminoxy)propyloxy hGH;
N^{δ40}-2-(O-(4-{4-(1,3-bis(mPEG(20k)ylaminocarbonyloxy)prop-2-yloxy)butyryl}-aminobutyl)oximino)ethyl hGH;
N^{δ40}-2-(O-(4-{3-(mPEG(20k)yloxy)propionyl}aminobutyl)oximino)ethyl hGH;
N^{δ40}-2-(O-(4-{4-(2,3-bis(mPEG(20k)yloxy)prop-1-yloxy)butyryl}aminobutyl)oximino)ethyl hGH;
N^{δ40}-2-(O-(4-{5-(mPEG(20k)yloxy-5-oxopentanoyl}aminobutyl)oximino)ethyl hGH;
N^{δ40}-3-({4-(2-(2-(2-(2-(4(1,3-bi s(mPEG(20k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(20k)ylaminocarbonyloxy)prop-2-yloxy)butylidene}-aminoxy)propyloxy hGH;
N^{δ40}-3-({4-(mPEG(20k)yloxy)butylidene}aminoxy)propyloxy hGH;
N^{δ40}-3-({4-(2,3-bis(mPEG(20k)yloxy)prop-1-yloxy)butylidene}aminoxy)propyloxy hGH;
N^{δ40}-3-({3-(mPEG(20k)yloxy)propylidene}aminoxy)propyloxy hGH;
N^{δ40}-2-(O-(2-(3-(2,3-bis(mPEG(20k)yloxy)propyloxy)propylamino)-2-oxoethyl)oximino)ethyl hGH;
N^{δ40}-3-({4-(2,3-bis(mPEG(20k)yloxy)prop-1-yloxy)propylidene}aminoxy)propyloxy hGH;
N^{δ40}-2-(O-(4-{4-(1,3-bis(mPEG(30k)ylaminocarbonyloxy)prop-2-yloxy)butyryl}aminobutyl)oximino)ethyl hGH;
N^{δ40}-2-(O-(4-{3-(mPEG(30k)yloxy)propionyl}aminobutyl)oximino)ethyl hGH;
N^{δ40}-2-(O-(4-{-4-(2,3-bis(mPEG(30k)yloxy)prop-1-yloxy)butyryl}aminobutyl)oximino)ethyl hGH;
N^{δ40}-2-(O-(4-{5-(mPEG(30k)yloxy-5-oxopentanoyl}aminobutyl)oximino)ethyl hGH;
N^{δ40}-3-({4-(2-(2-(2-(2-(4-(1,3-bis(mPEG(30k)ylaminocarbonyloxy)prop-2-yloxy)butyrylamino)ethoxy)ethoxy)ethoxy)ethoxy)butylidene}aminoxy)prop-1-yloxy hGH;
N^{δ40}-3-({4-(1,3-bis(mPEG(30k)ylaminocarbonyloxy)prop-2-yloxy)butylidene}aminoxy)propyloxy hGH;
N^{δ40}-3-({4-(mPEG(30k)yloxy)butylidene}aminoxy)propyloxy hGH;
N^{δ40}-3-({4-(2,3-bis(mPEG(30k)yloxy)prop-1-yloxy)butylidene}aminoxy)propyloxy hGH;
N^{δ40}-3-({4-(mPEG(30k)yloxy)propylidene}aminoxy)propyloxy hGH;
N^{δ40}-2-(O-(2-(3-(2,3-bis(mPEG(30k)yloxy)propyloxy)propylamino)-2-oxoethyl)oximino)ethyl hGH;
N^{δ40}-3-({4-(2,3-bis(mPEG(30k)yloxy)prop-1-yloxy)propylidene}aminoxy)propyloxy hGH;
N^{δ40}-3-({4-{(2,3-bis(mPEG(20k)yloxy)prop-1-yl)PEGyloxy}butylidene}aminoxy)propyloxy hGH;
N^{δ40}-2-((4 (4 ((2,3-bis(mPEG(20k)yl)propyl)PEGyloxy)butyrylamino)butyl)oximino)ethyl hGH;
N^{ε}141-[2-O-(4-(4-(1,3-bis(mPEG(20K)aminocarbonyloxy)-2-propyloxy)butyrylamino)butyl)oxyimino)ethyl]hGH;
N^{ε}141-[2-(O-(4-(4-(mPEG(30K)oxy)butyrylamino)-butyl)oxyimino)-ethyl]hGH;
N^{ε}141-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}141-[2-(2-(2,3-(mPEG(20K)yloxy)propyloxycarbonylamino)ethyloximino)ethyl]hGH;
N^{ε}141-[2-(O-(2-(2-(mPEG(40K)yloxy)ethylamino)-2-oxoethyl)oximino)ethyl]hGH;
N^{ε}141-[2-(3-(4-((1,3-bis(mPEG(30K)ylaminocarbonyloxy)-2-propyloxy)butylidene)aminoxy)propyloxyimino)ethyl]hGH;
N^{ε}141/40-[2-O-(4-(4-(1,3-bis(mPEG(20K)aminocarbonyloxy)-2-propyloxy)butyrylamino)butyl)oxyimino)ethyl]hGH;
N^{ε}141/40-[2-(O-(4-(4-(mPEG(30K)oxy)butyrylamino)-butyl)oxyimino)-ethyl]hGH;
N^{ε}141/40-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}141/40-[2-(2-(2,3-(mPEG(20K)yloxy)propyloxycarbonylamino)ethyloximino)ethyl]hGH;
N^{ε}141/40-[2-(O-(2-(2-(mPEG(40K)yloxy)ethylamino)-2-oxoethyl)oximino)ethyl]hGH;
N^{ε}141/40-[2-(3-(4-((1,3-bis(mPEG(30K)ylaminocarbonyloxy)-2-propyloxy)butylidene)aminoxy)propyloxyimino)ethyl]hGH;
N^{ε}40-[2-O-(4-(4-(1,3-bis(mPEG(20K)aminocarbonyloxy)-2-propyloxy)butyrylamino)butyl)oxyimino)ethyl]hGH;
N^{ε}40-[2-(O-(4-(4-(mPEG(30K)oxy)butyrylamino)-butyl)oxyimino)-ethyl]hGH;
N^{ε}40-(3-((4-(2-(2-(2-(2-(4-(1,3-bis(mPEG(20K)ylaminocarbonyloxy)-2-propyloxy)butyrylamino)ethoxy)ethoxy)ethoxy)ethoxy)butylidene)aminoxy)propyloxy)hGH;
N^{ε}40-[2-(2-(2,3-(mPEG(20K)yloxy)propyloxycarbonylamino)ethyloximino)ethyl]hGH;
N^{ε}40-[2-(O-(2-(2-(mPEG(40K)yloxy)ethylamino)-2-oxoethyl)oximino)ethyl]hGH;
N^{ε}40-[2-(3-(4-((1,3-bis(mPEG(30K)ylaminocarbonyloxy)-2-propyloxy)butylidene)aminoxy)propyloxyimino)ethyl]hGH;

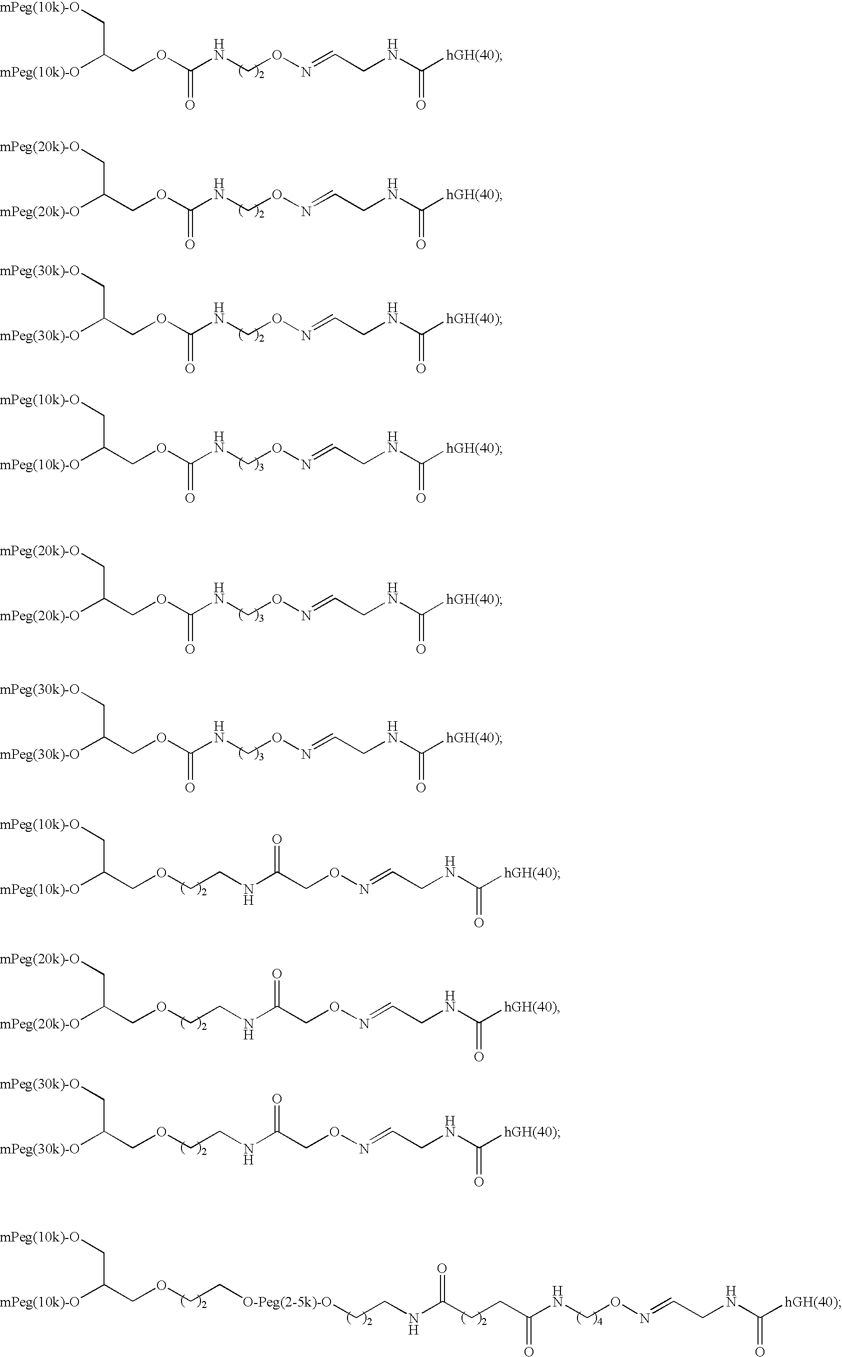

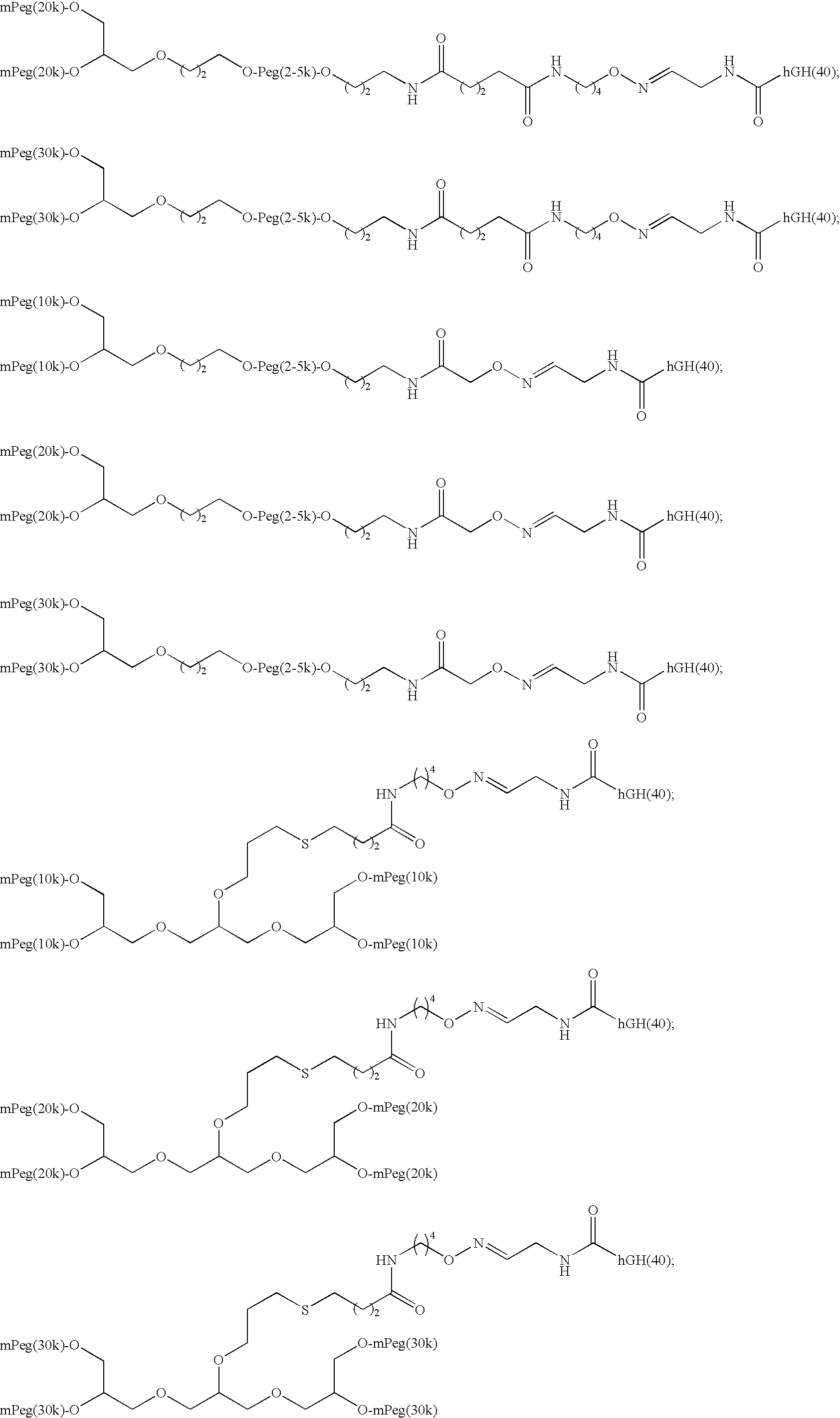

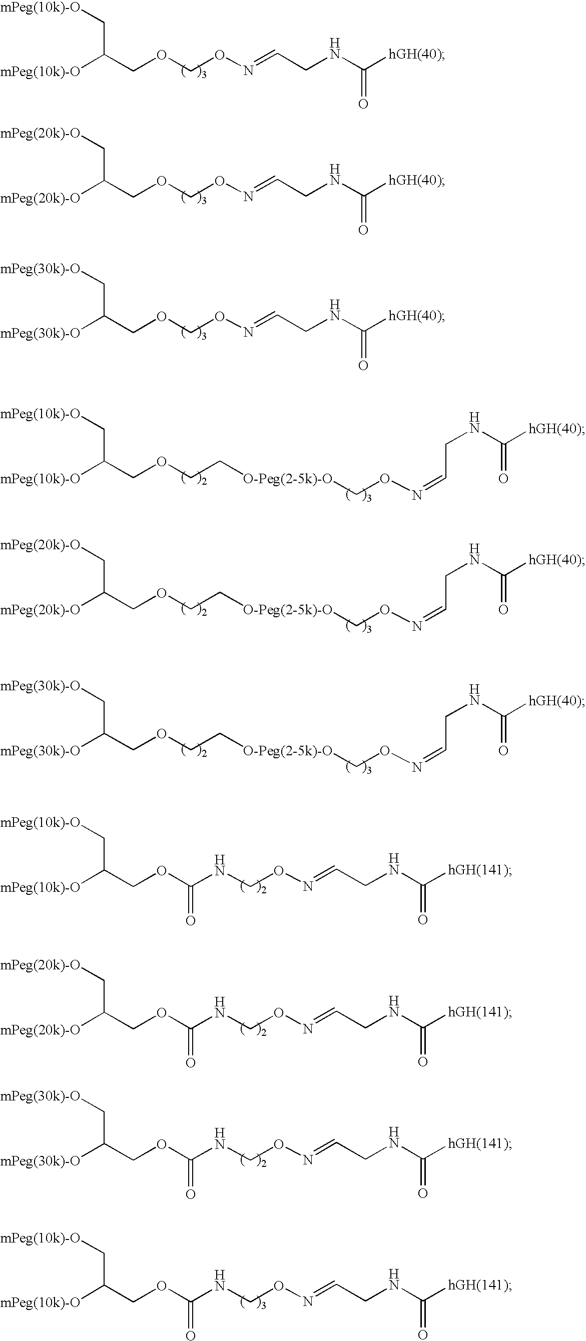

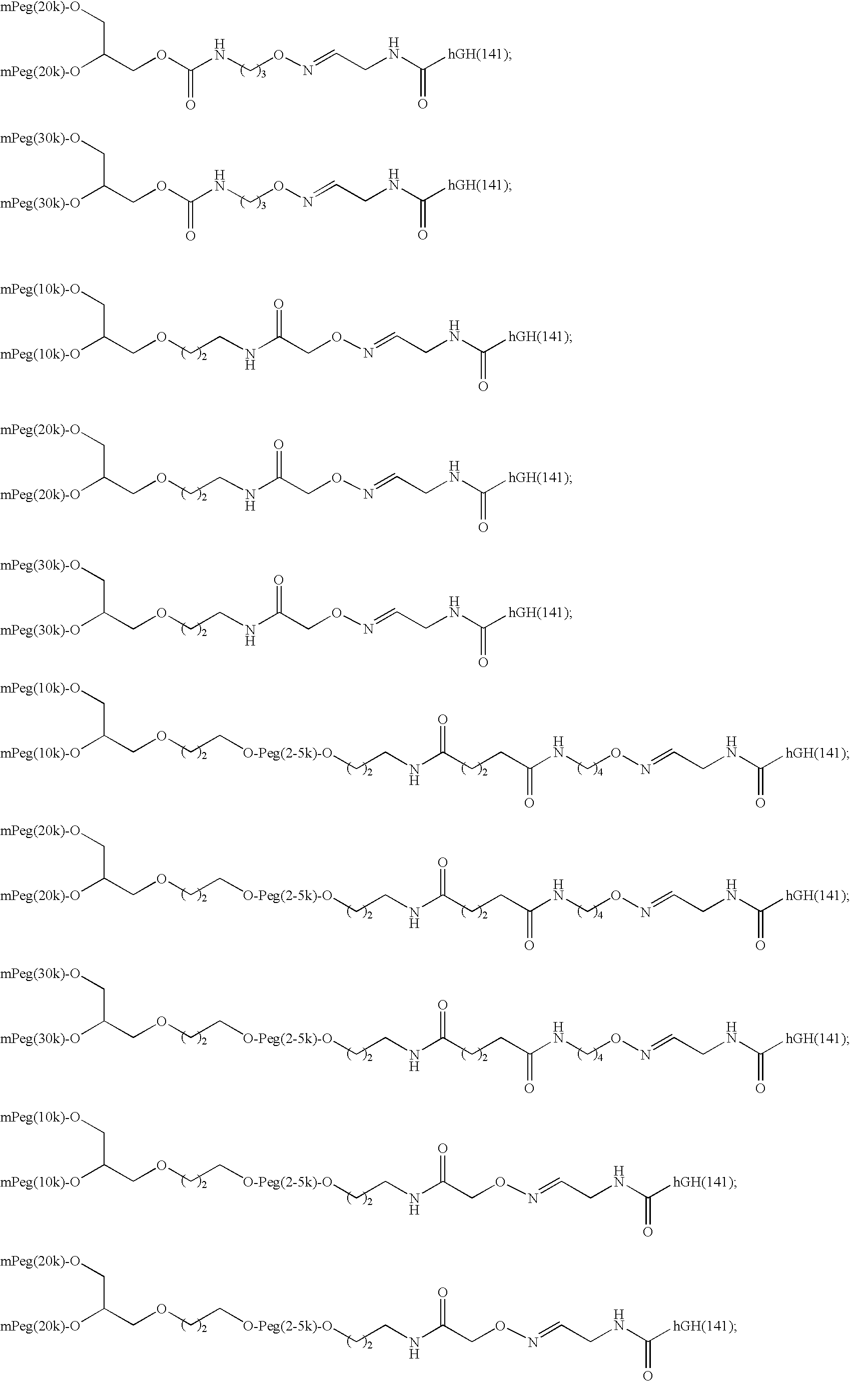

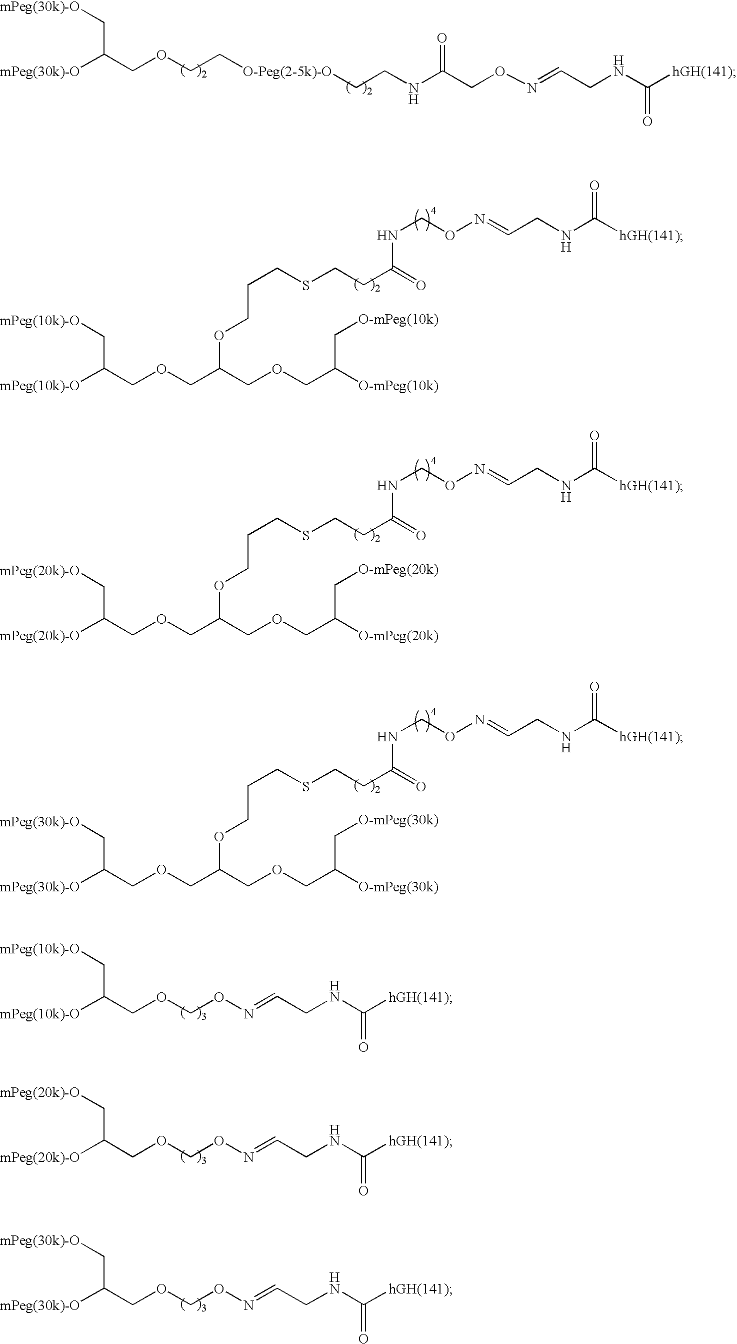

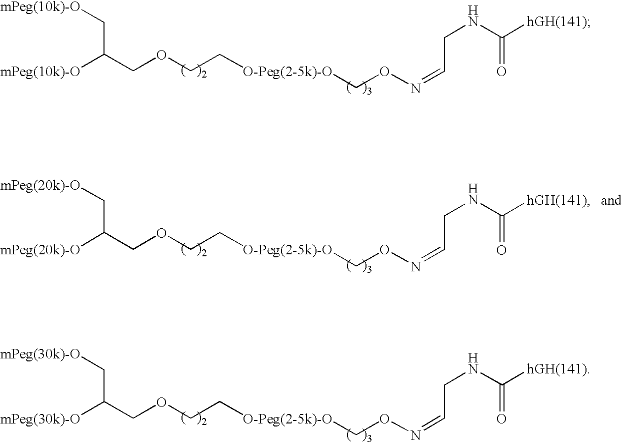

36. A compound according to formula [VI] 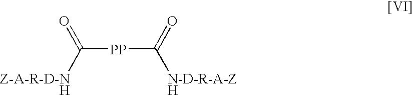
wherein PP represents a polypeptide radical obtained by removing —C(═O)—NH₂ from the side chain of two glutamine residues present in said polypeptide;
D represents —O— or a bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
A represents an oxime bond;
Z represents a moiety selected amongst 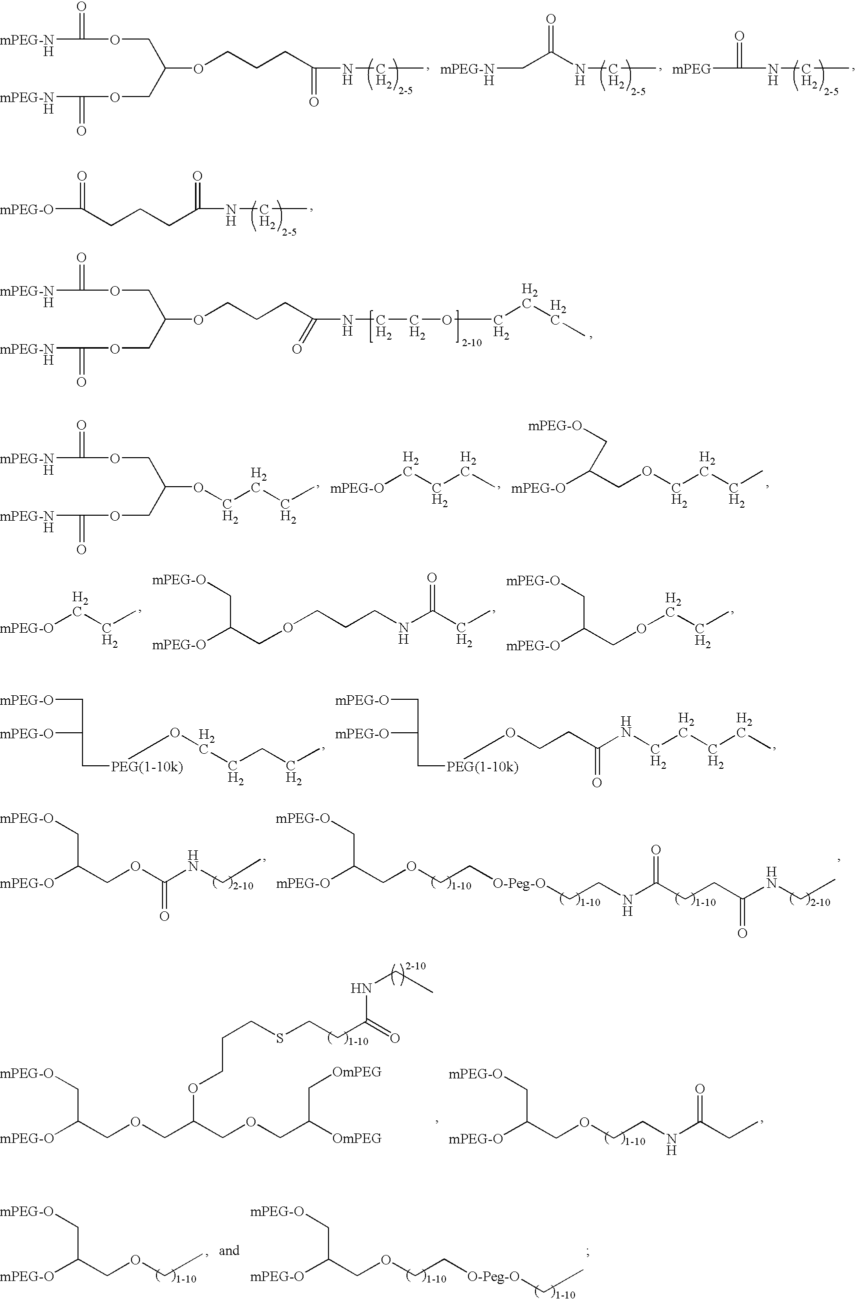
wherein, unless otherwise indicated, mPEG indicates an mPEG with a molecular weight between 5 kDa and 40 kDa and PEG indicates a PEG with a molecular weight between 1 kDa and 10 kDa;
and pharmaceutically acceptable salts, prodrugs or solvates thereof;
provided that if Z is 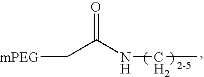
then mPEG is 10 kDa mPEG.

37. The compound according to claim 36, wherein D represents —O—.

38. The compound according to claim 36, wherein D represents a single bond.

39. The compound according to claim 36, wherein R represents —(CH₂)₄—CH(NH₂)—CO—NH—CH₂— or —(CH₂)₄—CH(NHCOCH₃)—CO—NH—CH₂—.

40. The compound according to claim 36, wherein R represents C₁₋₆alkylene.

41. The compound according to claim 40, wherein R represents C₁₋₃alkylene.

42. The compound according to claim 41, wherein R represents methylene or propylene.

43. The compound according to claim 36, wherein Z represents 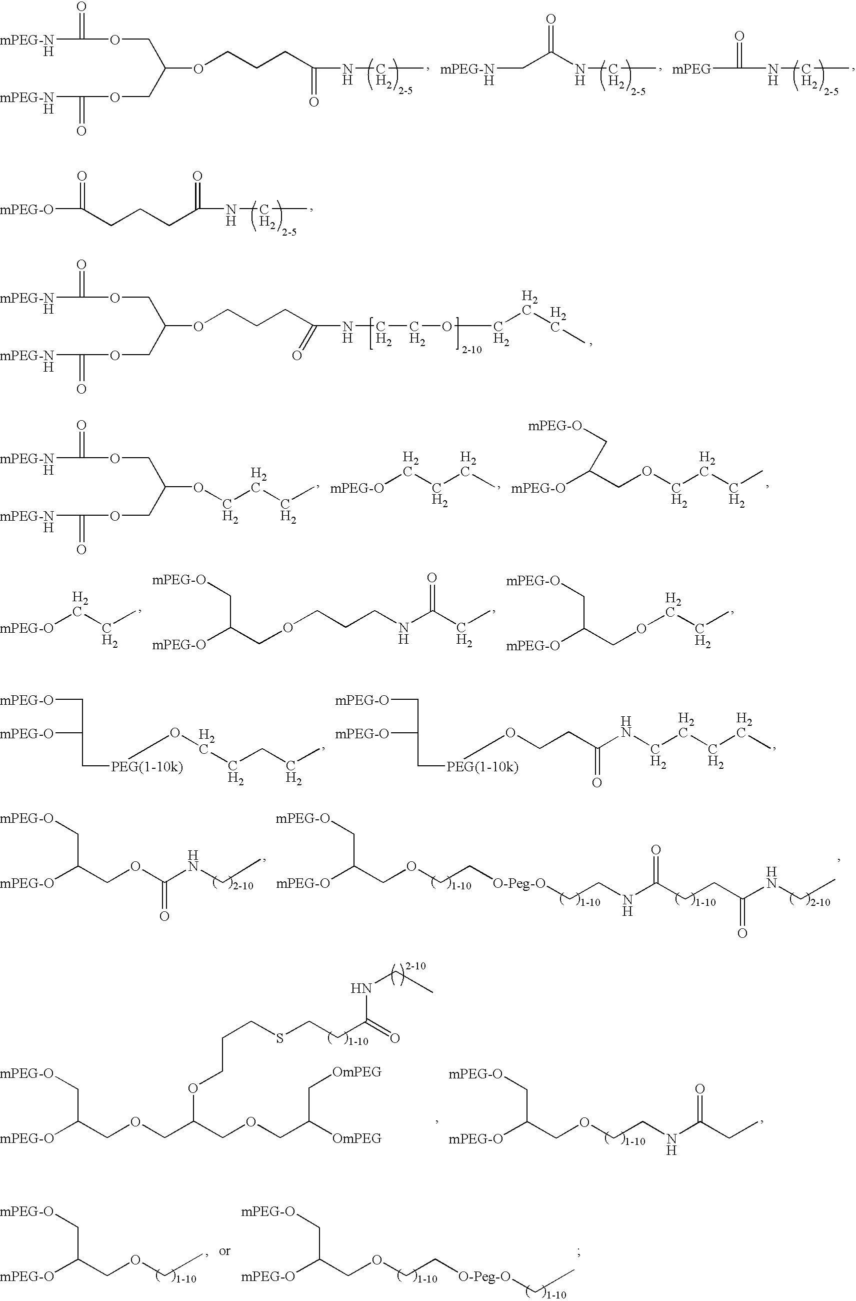
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 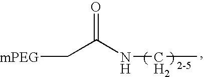
then PEG is 10 kDa PEG.

44. The compound according to claim 36, wherein the polypeptide is a glutamine residue comprising growth hormone and PP represents a growth hormone radical obtained by removing —C(═O)—NH₂ from the side chain of two glutamine residues present in the growth hormone.

45. The compound according to claim 44, wherein GH represents the radical obtained by removing —C(═O)—NH₂ from the side chain of two glutamine residues present in an hGH comprising the amino acid sequence of SEQ ID No. 1.

46. The compound according to claim 44, wherein GH represents the radical obtained by removing —C(═O)—NH₂ from the side chain of two glutamine residues present in 20 kDa hGH.

47. The compound according to claim 44, wherein GH represents the radical obtained by removing —C(═O)—NH₂ from the side chain of a glutamine residue in the position corresponding to position 40 in SEQ ID No. 1, and from the side chain of the glutamine residue in the position corresponding to position 141 in SEQ ID No. 1; or
GH represents the radical obtained by removing —C(═O)—NH₂ from the side chain of a glutamine residue in the position corresponding to position 40 in SEQ ID No. 1, wherein the glutamine residue in the position corresponding to position 141 in SEQ ID No. 1 has been deleted or substituted with another amino acid, and by removing —C(═O)—NH₂ from the side chain of another glutamine residue present in a position different from the positions corresponding to positions 40 and 141 in SEQ ID No. 1; or
GH represents the radical obtained by removing —C(═O)—NH₂ from the side chain of a glutamine residue in the position corresponding to position 141 in SEQ ID No. 1, wherein the glutamine residue in the position corresponding to position 40 in SEQ ID No. 1 has been deleted or substituted with another amino acid, and by removing —C(═O)—NH₂ from the side chain of another glutamine residue present in a position different from the positions corresponding to positions 40 and 141 in SEQ ID No. 1; or
GH represents the radical obtained by removing —C(═O)—NH₂ from the side chain of two glutamines present in an hGH in positions different from the positions corresponding to positions 40 and 141 in SEQ ID No. 1, wherein any glutamine residues present in the positions corresponding to positions 40 and 141 in SEQ ID No. 1 has been deleted or substituted with other amino acids.

48. A human growth hormone, which is covalently attached to a moiety comprising PEG, and in particular mPEG, wherein said PEG comprising moiety is attached to the side chain of glutamine residue 40, to the side chain of glutamine 141 or to the side chains of glutamine 40 and glutamine 141 of human growth hormone, provided it is not
N^{ε141}-[2-(4-(4-(mPEG(20k)ylbutanoyl)-amino-butyloxyimino)-ethyl]hGH,
N^{ε141}-[2-(1-(hexadecanoyl)piperidin-4-yl)ethyloxyimino)-ethyl]hGH,
N^{ε141}(2-(4-(4-(1,3-bis(mPEG(20k)ylaminocarbonyloxy)prop-2-yloxy)butyrylamino)butyloxyimino)ethyl)hGH,
N^{ε141}(2-(4-(2,6-bis(mPEG(20k)yloxycarbonylamino)hexanoylamino)butyloxyimino)ethyl)hGH,
N^{ε141}(2-(4-(4-(mPEG(30k)yloxy)butyrylamino)butyloxyimino)ethyl)hGH,
N^{ε141}(2-(4-(4-(mPEG(20k)yloxy)butyrylamino)butyloxyimino)ethyl)hGH, or
N^{ε141}(2-(4-(3-(mPEG(30k)yloxy)propanoylamino)butyloxyimino)ethyl)hGH.

49. (canceled)

50. (canceled)

51. (canceled)

52. (canceled)

53. (canceled)

54. A pharmaceutical composition comprising a polypeptide represented by formula [I] 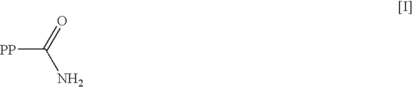
wherein PP represents a polypeptide radical obtained by removing —C(═O)—NH₂ from the side chain of a glutamine residue present in the polypeptide, with a nitrogen containing nucleophile of formula [II]
H₂N-D-R—X [II]
wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂ an aldehyde, a ketone, or a latent group which upon further reaction may be transformed into —O—NH₂, an aldehyde or a ketone;
in the presence of transglutaminase to form a transaminated polypeptide of formula [III] 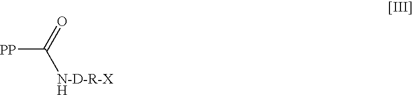
optionally, if X is a latent group, transforming said latent group into —O—NH₂ an aldehyde or a ketone,
said transaminated polypeptide being further reacted with a second compound of formula [IV]
Y—Z [IV]
wherein Y,
if X represents an aldehyde, a ketone, or a latent group which upon further reaction may be transformed an aldehyde or a ketone, represents —O—NH₂; or,
if X represents —O—NH₂, or a latent group which upon further reaction may be transformed into —O—NH₂, represents an aldehyde or a ketone; and
Z represents a moiety selected amongst 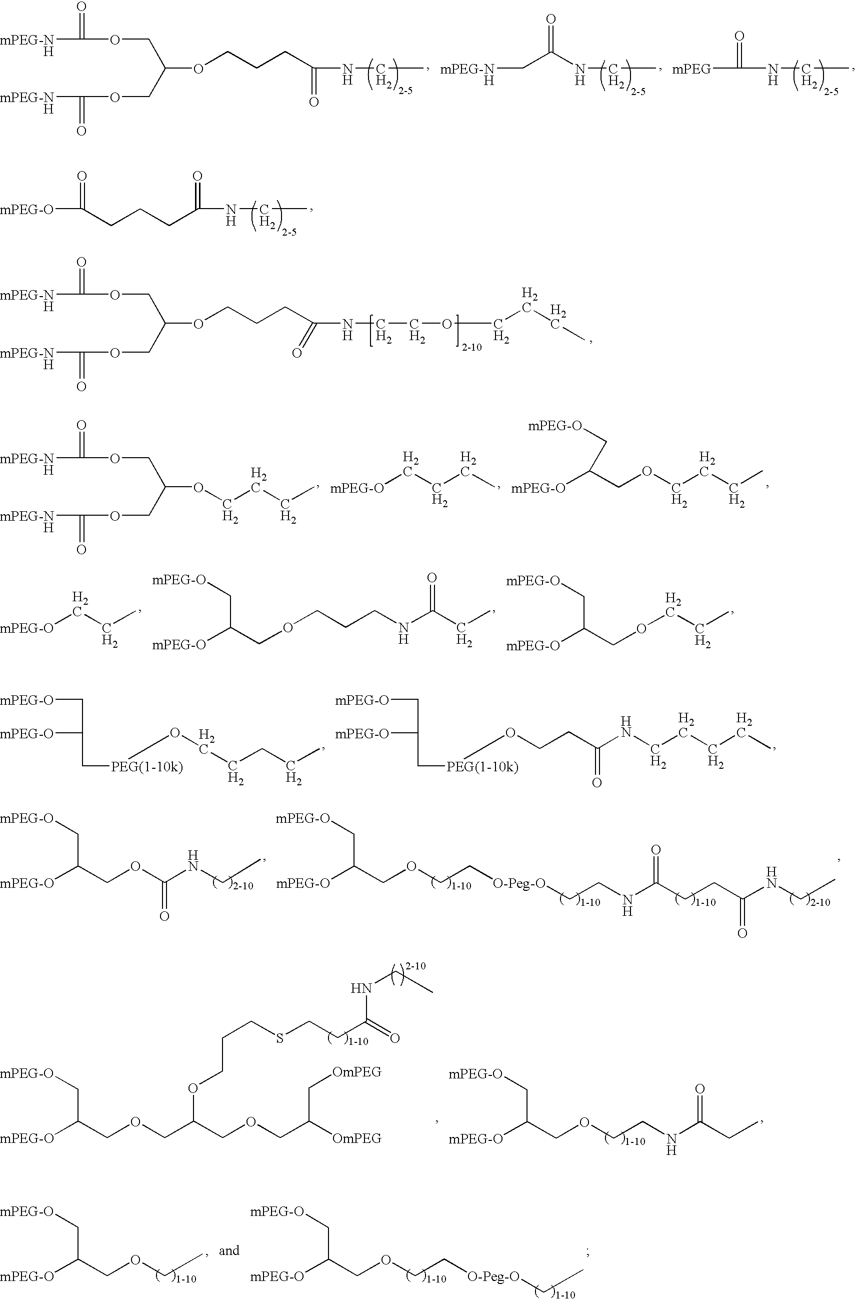
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 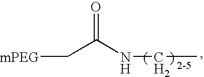
then PEG is 10 kDa PEG to form a PEGylated polypeptide of formula [V] 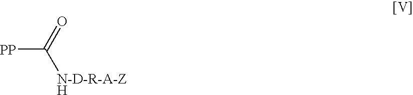
wherein A represents an oxime bond.

55. A method for treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1^{st} toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; short stature due to glucucorticoid treatment in children; for acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a polypeptide represented by formula [I] 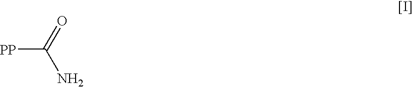
wherein PP represents a polypeptide radical obtained by removing —C(═O)—NH₂ from the side chain of a glutamine residue present in the polypeptide, with a nitrogen containing nucleophile of formula [II]
H₂N-D-R—X [II]
wherein D represents —O— or a single bond;
R represents C₁₋₆alkylene, —(CH₂)₄—CH(NH₂)—CO—NH—CH₂—, —(CH₂)₄—CH(NHCOCH₃)—CO—NH—CH₂—, or C₅₋₁₅heteroalkylene;
X represents —O—NH₂, an aldehyde, a ketone, or a latent group which upon further reaction may be transformed into —O—NH₂, an aldehyde or a ketone;
in the presence of transglutaminase to form a transaminated polypeptide of formula [III] 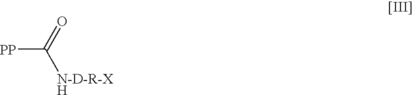
optionally, if X is a latent group, transforming said latent group into —O—NH₂, an aldehyde or a ketone,
said transaminated polypeptide being further reacted with a second compound of formula [IV]
Y—Z [IV]
wherein Y,
if X represents an aldehyde, a ketone, or a latent group which upon further reaction may be transformed an aldehyde or a ketone, represents —O—NH₂; or
if X represents —O—NH₂, or a latent group which upon further reaction may be transformed into —O—NH₂, represents an aldehyde or a ketone; and
Z represents a moiety selected amongst 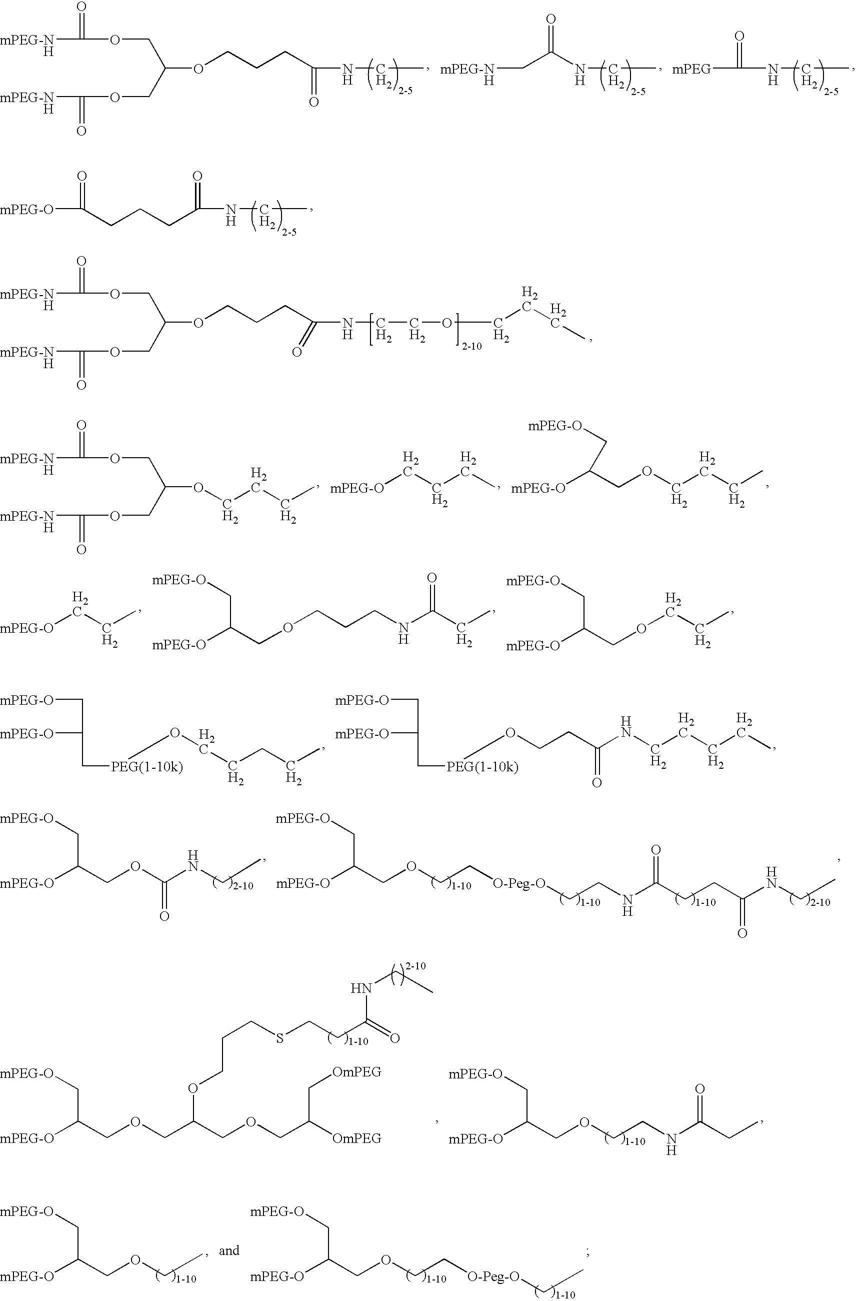
wherein, unless otherwise indicated, mPEG indicates a mPEG with a molecular weight of between 5 kDa and 40 kDa and PEG indicates a PEG with a molecular weight between 1 kDa and 10 kDa;
provided that if Z is 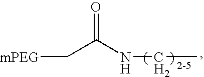
then PEG is 10 kDa PEG to form a PEGylated polypeptide of formula [V] 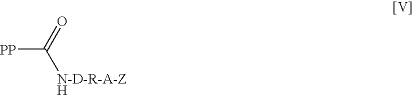
wherein A represents an oxime bond.

56. (canceled)


SEQUENCE LISTING (1 sequence)

SEQ ID NO: 1 - protein, 191 aa, Homo sapiens [sequence omitted]